(19) United States
(12) Patent Application Publication
(10) Pub. No.: US 2026/0183364 A1
(43) Pub. Date: Jul. 2, 2026

(54) CONTROL APOE LEVEL FOR TREATING FETAL ALCOHOL SPECTRUM DISORDERS

(71) Applicant: Children's National Medical Center, Washington, DC (US)

(72) Inventors: Kazue Hashimoto-Torii, Brookeville, MD (US); Hye Hwang, Eagan, MN (US); Masaaki Torii, Brookeville, MD (US)

(21) Appl. No.: 19/373,166

(22) Filed: Oct. 29, 2025

Related U.S. Application Data
(60) Provisional application No. 63/713,713, filed on Oct. 30, 2024.

(51) Int. Cl. A61K 38/10; A61K 9/00; A61P 25/28; G01N 33/92

(52) U.S. Cl. CPC A61K 38/10; A61K 9/0043; A61P 25/28; G01N 33/92; G01N 2333/775; G01N 2800/385; G01N 2800/52

(57) ABSTRACT

The present disclosure relates to methods for treating or alleviating a fetal alcohol spectrum disorder (FASD) in a subject, the method comprising administering to the subject an effective amount of an apolipoprotein E (APOE)-modulating therapeutic, thereby treating or alleviating the FASD.

### RELATED APPLICATIONS

The instant application claims priority to U.S. Provisional Application No. 63/713,713, filed on Oct. 30, 2024. The entire contents of the foregoing application are expressly incorporated by reference herein.

### GOVERNMENT SUPPORT

This invention was made with government support under AA026272 and AA027693 awarded by National Institute of Health. The government has certain rights in the invention.

### SEQUENCE LISTING

The application contains a Sequence Listing which has been submitted electronically in .XML format and is hereby incorporated by reference in its entirety. Said .XML copy, created on Jan. 26, 2026 is named “135372-02902.xml” and is 22,645 bytes in size. The sequence listing contained in this .XML file is part of the specification and is hereby incorporated by reference herein in its entirety.

### FIELD OF THE INVENTION

This present disclosure generally relates to methods for treating subjects having Fetal Alcohol Spectrum Disorders (FASD).

### BACKGROUND OF THE INVENTION

Fetal Alcohol Spectrum Disorders (FASD) include neurocognitive or neurobehavior deficits. The clinical presentation of FASD is heterogenous, including intellectual disability, delay in motor and language development, and other psychiatric and neurological problems. Currently, there are no effective treatments for FASD.

Apolipoprotein E (APOE) controls synaptic plasticity, which is crucial for brain function. The impact of prenatal alcohol exposure (PAE) on APOE function and the involvement of APOE in the pathogenesis of FASD have not been explored.

Accordingly, there is a need in the art for improved methods for treating, preventing, and alleviating FASD.

The foregoing Background description is for the purpose of generally presenting the context of the disclosure. Work of the inventors (if any), to the extent it is described in this background section, as well as aspects of the description which may not otherwise qualify as prior art at the time of the filing, are neither expressly or impliedly admitted as prior art against the present disclosure.

### SUMMARY OF THE INVENTION

The present application provides methods and compositions for treating subjects having FASD to improve neurocognitive or neurobehavior deficits by controlling the APOE level, such as by administering an APOE receptor agonist, such as COG 133. The present application discloses that prenatal alcohol exposure (PAE) can reduce APOE levels in the mouse brain and in the peripheral blood of humans and mice. The present application also discloses that: APOE receptor agonist administration can alleviate neurobehavioral, molecular, and electrophysiological phenotypes in mice which were prenatally exposed to alcohol. Chromatin accessibility at the ApoE locus is reduced by PAE. Carriers of the rs584007 single nucleotide polymorphism in the APOE enhancer, the chromatin of which is closed by PAE in mice, are susceptible to neurobehavioral deficits by PAE. Reduced plasma APOE level is associated with lower cognitive performance in infants prenatally exposed to alcohol.

In some aspects, provided herein is a method for treating or alleviating a fetal alcohol spectrum disorder (FASD) in a subject, the method comprising administering to the subject an effective amount of an apolipoprotein E (APOE)-modulating therapeutic, thereby treating or alleviating the FASD.

In some embodiments, the APOE-modulating therapeutic is selected from the group consisting of a retinoid X receptor (RXR) agonist, a liver X receptor (LXR) agonist, and an APOE-mimetic therapeutic. In some embodiments, the APOE-modulating therapeutic is an APOE-mimetic therapeutic.

In some aspects, provided herein is a method for treating or alleviating an FASD in a subject, the method comprising administering to the subject an effective amount of an APOE-mimetic therapeutic, thereby treating or alleviating the FASD.

In some embodiments, the FASD is selected from the group consisting of fetal alcohol syndrome (FAS), partial fetal alcohol syndrome (pFAS), alcohol-related neurodevelopmental disorder (ARND), and neurobehavioral disorder associated with prenatal alcohol exposure (ND-PAE).

In some embodiments, the APOE-mimetic therapeutic is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2. In some embodiments, the APOE-mimetic therapeutic is COG133.

In some embodiments, the subject carries a mutation in an APOE enhancer element. In some embodiments, the mutation is a single nucleotide polymorphism (SNP). In some embodiments, the SNP is rs584007. In some embodiments, rs584007 comprises the amino acid sequence of

(SEQ ID NO: 16)
AGGAGGGGCGTCAGAGGGTGAATAARAG
CAGATAGAGTGTTTGGGGGAGGT.

In some embodiments, the subject has low APOE plasma levels, as compared to APOE plasma levels of an individual without prenatal alcohol exposure (PAE). In some embodiments, the subject has low APOE plasma levels, as compared to APOE plasma levels of an individual without the FASD.

In some embodiments, treating or alleviating the FASD comprises treating or alleviating one or more symptoms selected from the group consisting of a learning deficit, motor deficit, anxiety, depression, poor reasoning and judgement, short attention span, hyperactive behavior, poor memory, and speech delay.

In some embodiments, the subject is a human subject. In some embodiments, the subject is a pediatric subject. In some embodiments, the subject is an infant. In some embodiments, the APOE-mimetic therapeutic is administered intranasally or intravenously. In some embodiments, the APOE-mimetic therapeutic is administered intranasally.

In some aspects, provided herein is a method of increasing APOE activity in a subject, the method comprising administering to the subject an effective amount of an APOE-modulating therapeutic, thereby increasing APOE activity, wherein the subject has or is suspected of having PAE.

In some aspects, provided herein is a method of identifying a subject having an FASD, the method comprising: (a) measuring the level of APOE in a biological sample from the subject, and (b) comparing the measured level of APOE to a control level, wherein a lower level of APOE in the biological sample from the subject compared to the control level is indicative of the subject having the FASD, optionally, the method further comprising selecting or recommending the subject for treatment of FASD with any of the preceding methods, and further optionally treating the subject having FASD with any of the preceding. In some embodiments, the biological sample is a plasma sample.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H show that prenatal alcohol exposure (PAE) reduces apolipoprotein E (APOE) expression in peripheral blood mononuclear cells (PBMCs). Specifically, FIG. 1A provides a schematic of PBMC RNA profiling following accelerated rotarod test. FIG. 1B provides a representative result of FACSorting of PBMCs collected from control animals at postnatal day (P) 35. PBMCs were labeled with CD11b, CD19, and CD90.2, and sequentially sorted based on the cell size (forward scatter [FSC] versus side scatter [SSC]) and singlets (FSC vs. trigger pulse width). Then cells were further divided into B-cells (CD19+/CD90.2−), T-cells (CD90.2+/CD19−), and monocytes (CD11+/CD19−) indicated by orange, red, and blue boxes, respectively. FIG. 1C shows the top 10 enriched gene ontologies (GOs) with Combined Scores in downregulated (blue) and upregulated (red) differentially expressed genes (DEGs) by PAE in B-cells. FIG. 1D provides a volcano plot that shows the DEGs by PAE in B-cells. Top 15 DEGs are labeled with their gene symbols. Blue and red indicate downregulated and upregulated genes, respectively. FIG. 1E provides a heatmap of hierarchical K-means clustering showing 6 distinctive clusters of DEGs by PAE in B-cells. DEGs in cluster 2 contain lipid metabolism-related GOs (FIG. 7C). FIG. 1F shows the result of Ingenuity Pathways Analysis (IPA) on the genes in cluster 2. Regulator effects network overlaid with top diseases and functions shows that decreased Apoe expression is associated with other genes that have reduced expression by PAE and fall in the same pathways. FIG. 1G shows a bubble plot of Pearson's correlation analysis between the gene expression level and learning index of animals (control: n=15, PAE: n=14). Red and blue bubbles indicate significant (P<0.05) positive and negative correlations, respectively. Grey bubbles indicate non-significant (P≥0.05) correlations. The size of the circle corresponds to the p-value. FIG. 1H demonstrates that PAE mice show significantly lower levels of plasma APOE than control mice (control: n=19; PAE: n=18). *P=0.024 by two-tailed Student's t-test. Data represent mean±s.e.m.

FIGS. 2A-2D show that an APOE receptor agonist (APOE-RA) improves motor learning in PAE mice. Specifically, FIG. 2A provides a Pearson's correlation analysis that reveals a positive correlation (R2=0.558, P=0.0166) between the motor learning index and the number of APOE positive cells in the motor cortex in PAE mice (see FIGS. 9A-9H). Each dot represents an individual animal (n=8). Solid line indicates the best fit line. FIG. 2B provides a schematic that shows a timeline of the APOE-RA treatment and behavioral tests. FIG. 2C demonstrates that PAE mice treated daily with APOE-RA for 10 days show significantly improved terminal speed in trial 6 in accelerated rotarod test compared to vehicle-treated PAE mice (control+vehicle: n=9; control+APOE-RA: n=10; PAE+vehicle: n=7; PAE+APOE-RA: n=10). ***P<0.001 by two-way analysis of variance (ANOVA) with simple main effect test. Data represent mean±s.e.m. FIG. 2D shows that postnatal APOE-RA treatment significantly improves motor learning index in PAE mice (control+vehicle: n=9; control+APOE-RA: n=10; PAE+vehicle: n=7; PAE+APOE-RA: n=10). Box plot represents 25th, median, and 75th percentile. Whiskers extended to min and max values. **P<0.01 by two-way ANOVA with simple main effect test. Data represent mean±s.e.m.

FIGS. 3A-3K show that postnatal APOE-RA treatment alleviated the decreased endogenous APOE level, excessive potassium intermediate/small calcium-activated channel, subfamily N, member 2 (KCNN2), and shorter decay time of NMDA receptor (NMDAR)-mediated spontaneous excitatory postsynaptic currents (sEPSCs) in the motor cortex in PAE mice. Specifically, FIG. 3A shows that APOE-RA treatment increases the number of APOE-positive cells that is reduced by PAE in layer V/VI of the motor cortex (n=4 per group). **P<0.01 by two-way ANOVA with simple main effect test. Data represent mean±s.e.m. FIG. 3B provides representative images of APOE (magenta, arrowheads) and DAPI (blue) staining in layer V/VI of the motor cortex in PAE mice treated with vehicle or APOE-RA. Dotted boxes indicate the region shown at higher magnification in the inset. The number of KCNN2 puncta is significantly decreased in layer II/III (FIG. 3C) and layer V/VI of the motor cortex (FIG. 3E) in PAE mice after the APOE-RA treatment compared to vehicle treatment (n=6 per group). **P<0.01, ***P<0.001 by two-way ANOVA with simple main effect test. Data represent mean±s.e.m. FIGS. 3D and 3F provide representative images of KCNN2 (red, arrowheads) and DAPI (blue) staining. Scale bars=10 μm. FIG. 3G provides examples of NMDAR-mediated sEPSCs recorded under the voltage-clamp condition at a holding potential of +50 mV in pyramidal neurons of layer V/VI in the indicated experimental groups. FIG. 3H shows that NMDAR-mediated sEPSCs averaged from 5 events in control, PAE, and PAE+APOE-RA mice. FIGS. 3I-3K show that no significant differences are observed in the amplitude (FIG. 3I) and rise time (FIG. 3J) between the groups. PAE group shows a significantly shorter decay time than control and APOE-RA treated PAE group (FIG. 3K). Box plot represents the 25th, median, and 75th percentile. n=8 cells per group. Whiskers extend to min and max values. *P<0.05 by One-way ANOVA with Tukey's post hoc test.

FIGS. 4A-4F show that PAE decreases chromatin accessibility in the brain. FIG. 4A shows that PAE mice show a significantly increased corticosterone concentration in the plasma compared to control mice (control: n=12, PAE: n=9). *P<0.05 by two-tailed Student's t-test. Data represent mean±s.e.m. FIG. 4B shows distribution of ATAC-seq fragment lengths demonstrating enrichment under 100 bp and around 200 bp, indicating nucleosome-free and mono-nucleosome-bound fragments, respectively. FIG. 4C provide peak annotation graphs showing that the proportion of aligned genomic features is similar between control and PAE samples. FIG. 4D provide heatmaps showing the enrichment of ATAC reads around the transcription start site (TSS) (−1,000-+1,000) in both control and PAE mouse samples. ATAC-seq signal in regulatory regions of the Apoe (FIG. 4E) and the quantification (FIG. 4F) show significantly reduced chromatin accessibility in the promoter, 3′ untranslated region (UTR), and multienhancer (ME) in the PAE group. 5′ UTR shows a trend of decrease in the PAE group (control: n=2, PAE: n=4). *P<0.05, **P<0.01, ***P<0.001 by two-tailed Student's t-test. The box plot represents the 25th, median, and 75th percentile. Whiskers extend to the min and max values. Each dot represents an individual animal.

FIGS. 5A-5F show that the single nucleotide polymorphism (SNP) rs584007 in an ApoE enhancer is associated with delayed matching to sample task (DMS) Z-score in PAE children. FIG. 5A provides a table showing information on study subjects. No significant differences are found in the gender ratio and mean age between the three ancestry groups by Chi-square test and one-way ANOVA, respectively. A significant difference in fetal alcohol syndrome (FAS) diagnosis, but not in the number of PAE individuals, is observed between the ancestry groups by Chi-square test. NA=not applicable. FIG. 5B provides a regional plot between 45.4 and 45.44 Mb on chromosome 19 around the APOE locus in the African (aa), American Hispanic (amr), and European (ea) ancestries and meta-analysis (metal) of GWAS with DMS Z-score. The interaction between SNP and PAE shows a significant association with the DMS z-score in the group of African American ancestry. FIG. 5C provides a table indicating the frequency of individuals with the rs584007 genotype in the African American ancestry group. FIG. 5D shows that in the group of African American ancestry, AA (genotype with a homozygous for rs584007 SNP) individuals prenatally exposed to alcohol have significantly lower DMS Z-scores compared to individuals with other genotypes with or without exposure to alcohol. *P<0.05, **P<0.01 by two-way ANOVA with simple main effect test. Data represent mean±s.e.m. FIG. 5E provides a table indicating the frequency of individuals with the rs584007 genotype in all ancestry groups. FIG. 5F shows that when all ancestry groups are combined, no significant interaction between PAE and SNP is observed by two-way ANOVA (P=0.3769). A significant alcohol effect is observed (P<0.0001). There is a marginal difference in the DMS Z-score between AA and other genotypes in alcohol exposed groups (AA vs GG P=0.09937; AA vs AG P=0.0991 by Tukey's post hoc test). Data represent mean±s.e.m.

FIGS. 6A-6C show that APOE in the plasma is reduced in PAE patients. FIG. 6A shows baseline characteristics of mothers who drank alcohol during pregnancy (users) and who did not (nonusers) and their children. Right columns show the results of Inverse probability weighing analysis. FIG. 6B provides a graph showing that the plasma APOE level at age 2-4 years old is significantly lower in PAE children. *P<0.05 by Student's t-test. Data represent mean±s.e.m. FIG. 6C provides Pearson's correlation analysis showing positive correlations between the plasma APOE level and the Bayley Scales of Infant Development (BSID-II) Mental Development Index (MDI) score at 12 months old, but not at 6 months old.

FIGS. 7A-7G show determination of the optimal cluster number for K-means clustering of B-cell DEGs and GOs enriched in each cluster. FIG. 7A provides a within sum of squares (WSS) plot suggesting the optimal number of K-means clustering for DEGs in B-cells to be six. FIGS. 7B-7G show GOs enriched in indicated clusters are determined by Enrichr. Only statistically significant GOs are shown in the figures.

FIGS. 8A-8B provide T-cell RNA profiling revealing the impact of PAE on lipid-related pathways. FIG. 8A shows GOs enriched in the genes downregulated in T-cells are identified by Enrichr. No GOs are significantly enriched in the upregulated genes (data not shown). The top regulator effect networks overlaid with diseases and disorders demonstrated that the activity of lipid synthesis is expected to be increased in T-cells by PAE (figure not shown). FIG. 8B shows that there are 116 and 6565 differentially expressed genes in B-cells and T-cells, respectively, in PAE mice compared to the control mice. There are 36 overlapping genes, and Apoe is one of them. Venn diagram of the differentially expressed genes from B-cells and T-cells with a list of shared genes are shown.

FIGS. 9A-9H show that APOE is decreased in the motor cortex. FIG. 9A shows that the number of APOE expressing cells in the motor cortex is reduced in PAE mice compared to control mice at P30. No significant difference was observed in the cingulate cortex. Control: n=8; PAE: n=8. *P=0.026, n.s=not significant by two-tailed Student's t-test. Data represent mean±s.e.m. FIG. 9B shows representative images of APOE (magenta) and DAPI (blue) staining in layer V/VI of the motor cortex in control and PAE mice. Scale bar=30 μm. FIG. 9C shows representative images of NeuN (green), APOE (magenta), and DAPI (blue) staining in layer V/VI of the motor cortex in control and PAE mice at P30. Arrowheads point at the APOE expressing cells. Scale bar=30 μm. FIG. 9D provides graphs showing quantification of APOE staining intensity in each NeuN-positive cells (n=30 from 3 animals per group), indicating a reduction of APOE immunolabeling in the cortex of PAE mice. Each dot in the bar graph (left) and each bar in the histogram (right) represents an individual cell. Creation of simulated datasets was found in Material and Methods. Cohen's d was calculated to determine the effect size based on the difference between the means of data from control mice and the other datasets (PAE mice and two simulation models). ****P<0.0001 by two-tailed Student's t-test. Data represent mean±s.e.m. FIG. 9E provides representative images of GFAP (green), APOE (magenta), and DAPI (blue) staining in layer V/VI of the motor cortex in control and PAE mice at P30. Arrowheads point at the APOE expressing cells. Scale bar=30 μm. FIG. 9F provides graphs showing quantification of APOE staining intensity in each GFAP-positive cells (n=25 from 3 animals per group), indicating a reduction of APOE immunostaining intensity in the cortex of PAE mice. Each dot in the bar graph and each bar in the histogram represents an individual cell. Creation of simulated datasets was found in Material and Methods. Cohen's d was calculated to evaluate the effect size based on the difference between the means of data from control mice and the other datasets (PAE mice and two simulation models). ****P<0.0001 by two-tailed Student's t-test. Data represent mean±s.e.m. FIG. 9G provides representative images of GFAP (green) and DAPI (blue) staining in layer V/VI of the motor cortex in control and PAE mice at P30. Scale bar=50 μm. FIG. 9H shows quantification revealing no significant difference in the number of GFAP-positive cells in the motor cortex between control and PAE mice at P30 (n=8 per group). n.s=not significant by two-tailed Student's t-test. Data represent mean±s.e.m.

FIGS. 10A-10G show that APOE-RA administered by intraperitoneal injection reaches the brain. FIGS. 10A and 10C provide graphs showing colocalization of biotin and LRP1 staining in the motor cortex of control and PAE mice that were administered biotinylated APOE-RA intraperitoneally. Vehicle only was administered as the control (n=4 per group). Brains were collected 5 minutes post administration at P20. *P<0.05 by two-tailed Student's t-test. Data represent mean±s.e.m. FIGS. 10B and 10D show representative images of biotin (magenta), LRP1 (green), and DAPI (blue) staining. Arrowheads pointing at the biotin staining in LRP1-positive cell. Scale bar=5 μm. FIG. 10E shows a higher magnification image of biotin and LRP1 double labeling with orthogonal projection. Scale bar=5 μm. FIG. 10F provides Pearson's correlation analysis revealing no significant correlation (R2=0.031, P=0.287) between the intensities of biotinylated APOE-RA detection and immunolabeling of endogenous APOE in each cell in the motor cortex of PAE mice at P20. Brains were fixed 5 minutes after the last injection of biotinylated APOE-RA. Each dot represents an individual cell (n=39 from 3 animals). FIG. 10G shows representative images of APOE (green), biotin (magenta), and DAPI (blue) staining. Scale bar=10 km.

FIGS. 11A-11J show that postnatal APOE-RA treatment mitigates anxiety behavior of PAE mice. FIG. 11A shows that significant colocalization of biotin and LRP1 labeling is found in the cingulate cortex of control and PAE mice 5 minutes after intraperitoneal administration of biotinylated APOE-RA at P20. Vehicle only was administered as the control (n=4 per group). *P<0.05, **P<0.01 by two-tailed Student's t-test. Data represent mean±s.e.m. FIG. 11B shows representative images of biotin (magenta), LRP1 (green), and DAPI (blue) staining. Arrowheads indicate the biotin labeling within LRP1-positive cells. Scale bar=5 μm. FIG. 11C shows APOERA-treated PAE mice spend significantly more time in the open arm compared to vehicle-treated PAE mice (control+vehicle: n=9; control+APOE-RA: n=10; PAE+vehicle: n=7; PAE+APOE-RA: n=9). Elevated plus maze (EPM) tests were done around P30. *P<0.05 by two-way ANOVA with simple main effect test. Data represent mean±s.e.m. FIG. 11D shows representative images of line tracks in the EPM test. A dashed box outlines an open arm, and a solid box outlines a closed arm. FIG. 11E shows that APOE-RA treatment increases the number of cells positive for endogenous APOE expression in the cingulate cortex in PAE mice (n=4 per group). *P<0.05 by two-way ANOVA with Bonferroni's multiple comparison test. Data represent mean±s.e.m. FIG. 11F shows representative images of APOE immunolabeling (magenta) in the cingulate cortex in the PAE mice treated with vehicle or APOERA. Cell nuclei were labeled with DAPI (blue). Dotted boxes indicate the region shown in the inset at higher magnification. Arrowheads indicate the APOE-positive cells. Scale bar=100 μm. FIG. 11G The number of KCNN2 puncta is significantly decreased in the cingulate cortex of PAE mice after the APOE-RA treatment compared to vehicle treatment (n=6 per group). **P<0.01 by two-way ANOVA with Bonferroni's post hoc test. Data represent mean±s.e.m. FIG. 11G shows representative images of KCNN2 (red) and DAPI (blue) staining. The white arrowheads point at the KCNN2 punctae-containing cells. Scale bar=10 μm. FIG. 11I shows representative images of KCNN2 (red), PSD95 (green), and DAPI (blue) staining in the cingulate cortex of indicated groups of mice. Scale bar=10 μm. FIG. 11J shows that the colocalization of KCNN2 and PSD95 is significantly reduced in the cingulate cortex of PAE mice after the APOE-RA treatment for 10 days (n=6 per group). ****P<0.0001 by two-way ANOVA with simple main effect test. Data represent mean±s.e.m.

FIGS. 12A-12F show that the blood-brain barrier (BBB) is intact in the brains of PAE mice, and APOERA treatment does not affect the body weight. FIG. 12A shows an image of a P30 mouse 30 minutes post injection of sodium fluorescein. FIG. 12B provides representative images of brain slices showing no changes in BBB permeability to sodium fluorescein in PAE mice compared to control mice (n=6 per group). Scale bar=1 mm. FIG. 12C shows images of P30 mice 30 minutes post injection of Evans blue dye (left) and without injection (right). FIG. 12D shows that control and PAE mice injected with Evans blue dye do not show any difference in BBB permeability to the dye (n=2 per group). Scale bar=200 μm. FIGS. 12E-12F show that there is no statistically significant difference in the body weight between control and PAE groups regardless of the treatment type (vehicle or APOE-RA) (Male: control+vehicle: n=4, control+APOE-RA: n=5, PAE+vehicle: n=4, PAE+APOE-RA: n=5; Female: control+vehicle: n=5, control+APOE-RA: n=5, PAE+vehicle: n=3, PAE+APOE-RA: n=5). Two-way repeated measures ANOVA with post hoc simple main effect test (FIG. 12E) or Tukey's test (FIG. 12F). Data represent mean±s.e.m.

FIGS. 13A-13I show that PAE does not affect the expression level of GluN1, LRP1, and PSD95 in layer V/VI of the mouse motor cortex at P30. FIGS. 13A, 13C, and 13E show that the number of GluN1 (FIG. 13A) and LRP1 (FIG. 13C)-positive cells and the PSD95 (FIG. 13E) puncta are not changed in PAE mice compared to control mice [n=4 per group (a); control: n=4, PAE: n=6 (FIG. 13C), or n=6 per group (FIG. 13E)]. n.s=not significant by two-tailed Student's t-test. Data represent mean±s.e.m. FIGS. 13B, 13D, and 13F show representative images of GluN1 (FIG. 13B), LRP1 (FIG. 13D), and PSD95 (FIG. 13F) immunohistochemistry (green), respectively. Nuclei were labeled with DAPI (blue). Scale bars=m (FIGS. 13B and 13D) and 10 μm (FIG. 13F). FIGS. 13G and 13H show that the colocalization of KCNN2 and PSD95 is significantly reduced in the motor cortex of PAE mice after the APOE-RA treatment (n=6 per group). **P<0.01, ***P<0.001 by two-way ANOVA with simple main effect test. Data represent mean±s.e.m. FIG. 13I shows representative images of KCNN2 (red), PSD95 (green), and DAPI (blue) staining in layer V/VI of the motor cortex in indicated groups. Scale bar=10 μm.

FIG. 14 shows that phosphorylation of CREB is reduced in the motor cortex but not in the striatum and cerebellum in PAE mice at P30. Images show the bands of pan-CREB (top), phospho-CREB (middle) and GAPDH (bottom) extracted from the motor cortex of the indicated animal groups. The side lanes are for the molecular weight marker. Graphs show the ratio of the signal intensity between phospho-CREB and pan-CREB [n=3 samples each (two males and one female from different litters)]. Unpaired t-test. Data represent mean±s.e.m.

FIGS. 15A-15J show that PAE decreases Apoe mRNA expression level in the motor cortex without changes in Grin2a and Grin2b expression levels. FIG. 15A demonstrates that the motor cortex in PAE mice shows a significant reduction in Apoe mRNA expression compared to control mice at P30. Postnatal treatment with APOE-RA does not significantly alter the expression (n=8 per group). *P<0.05 by two-way ANOVA followed by Tukey's post hoc test. Data represent mean±s.e.m. FIG. 15B shows there is no significant difference in the Apoe mRNA expression level between indicated groups in the cingulate cortex (n=4 per group). n.s=not significant by two-way ANOVA followed by Tukey's post hoc test. Data represent mean±s.e.m. FIG. 15C shows representative RNAscope images probed with Apoe (green) with DAPI labeling (blue) in the motor and cingulate cortices of the indicated groups. Scale bar=5 μm. FIG. 15D shows a representative RNAscope image stained with a negative control probe (green) with DAPI labeling (blue) in the motor cortex. FIGS. 15E-15F show there are no changes in the Grin2a and Grin2b expression and their ratio in layer II/III of the motor cortex between control and PAE mice (n=3 per group). n.s=not significant by two-tailed Student's t-test. Data represent mean±s.e.m. FIG. 15G shows representative RNAscope images probed with Grin2a (green) and Grin2b (blue) in layer II/III of the motor cortex. Scale bar=20 μm. FIGS. 1511-15I shows that there are no changes in the Grin2a and Grin2b expression or their ratio in layer V/VI of the motor cortex between control and PAE mice (n=3 per group). n.s=not significant by two-tailed Student's t-test. Data represent mean±s.e.m. FIG. 15J shows representative RNAscope images probed with Grin2a (green) and Grin2b (blue) with DAPI labeling (blue) in layer V/VI of the motor cortex. Scale bar=20 μm.

FIGS. 16A-16D show that COG-133 improves impaired motor skill learning in PAE mice. FIG. 16A shows the experimental time course. Mice were prenatally exposed to alcohol (4 g/kg) at E16 and E17, and the effect of intranasal administration of COG-133 (1 or 5 mg/kg) for 10 days (red arrows) after 6 trials on the accelerated rotarod (blue arrows) was analyzed. FIG. 16B shows that the interaction between the effects of treatment [COG-133 or Vehicle (PBS)] and trial on the performance in the rotarod test is significant in PAE mice (assessed by two-way repeated measures ANOVA). COG-133-treated PAE mice show significantly shorter latency to fall from later trials; *P<0.05 by Tukey's HSD test. No significant effects of COG-133 were observed in the control mice (by two-way repeated measures ANOVA). The number in each parenthesis indicates the number of animals in each experimental group (males and females together). FIG. 16C demonstrates that COG-133-treated PAE mice show significant improvement in learning index from trial 7 to 12 compared to vehicle-treated PAE mice. Learning index was calculated by difference of latency to fall between each trial. **P<0.01 by one-way Tukey's HSD test. FIG. 16D demonstrates that COG-133-treated PAE mice show significant improvement in terminal speed from trial 7 to 12 compared to vehicle-treated PAE mice. Gray and black lines show the data for individual mice and the means, respectively. **P<0.01 Tukey's HSD test. Data represent mean±SEM.

### DETAILED DESCRIPTION OF THE INVENTION

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context.

The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

All publications, patents, and patent applications mentioned in this specification are hereby incorporated by reference in their entirety for all purposes, as if each individual publication, patent, and patent application were specifically and individually incorporated by reference.

### Overview

Fetal alcohol spectrum disorders (FASD) is an umbrella term for neurodevelopmental disorders caused by prenatal alcohol exposure (PAE). The clinical presentation of FASD is heterogenous, including intellectual disability, delay in motor and language development, and other psychiatric and neurological problems (Mattson et al. Alcohol Clin Exp Res. 2019; 43:1046-62). According to the Centers for Disease Control and Prevention, about 10% of women consume alcohol while pregnant in the US, suggesting that the estimated prevalence of FASD in 1-5% (weighted estimate of 3.1-9.85%) in school-aged children is conservative (May et al. JAMA. 2018; 319:474). Studies have suggested that the manifestations of FASD are due to multifaceted changes caused by the epigenetic effect of alcohol (Portales-Casamar et al., Epigenetics Chromatin. 2016; 9:25).

While PAE causes FASD, not all exposures result in the same FASD outcomes. For instance, only 4.3% of children with heavy exposure to ethanol develop fetal alcohol syndrome (FAS), the most severe form of FASD (Popova et al., Lancet Glob Health. 2017; 5:e290-e299; Abel et al., Neurotoxicol Teratol. 1995; 17:437-43). Furthermore, twin studies revealed that fraternal twins have vastly different FASD outcomes whereas identical twins exhibit similar FASD outcomes (Streissguth et al., Am J Med Genet. 1993; 47:857-61; Hemingway et al., Adv Pediatr Res. 2018; 05:23). This evidence suggests that genetic factors modify fetal susceptibility to PAE. However, a lack of understanding of predisposing genetic factors that interact with alcohol exposure and a lack of knowledge about epigenetic effects of PAE have served as an impediment to developing effective treatment for neurocognitive problems in FASD.

Herein, a novel mechanism of treatment for patients with PAE involving the gene that encodes the protein apolipoprotein E (APOE) was discovered. APOE controls synaptic plasticity, which is crucial for brain function (Lane-Donovan et al., Trends Endocrinol Metab. 2017; 28:273-84). For instance, Apoe-deficient mice show a decreased number of synapses in the brain, including the frontal cortex and hippocampus, without obvious gross structural abnormalities (Masliah et al., Exp Neurol. 1995; 136:107-22; Lane-Donovan et al., J Neurosci. 2016; 36:10141-50; Tensaouti et al., ENeuro. 2018; 5:ENEURO.0155-18.2018). APOE receptors such as low-density lipoprotein receptor related protein 1 (LRP1), are localized at the postsynaptic membrane, and abolishing these receptors in neurons results in impaired motor function (Liu et al., J Neurosci. 2010; 30:17068-78). However, the impact of PAE on APOE function and the involvement of APOE in the pathogenesis of FASD has never been explored.

The invention disclosed herein is partly based on the discovery that reduction of Apolipoprotein E (APOE) is critically involved in neurobehavioral deficits in FASD. It is shown that prenatal alcohol exposure (PAE) changes chromatin accessibility of Apoe locus, and causes reduction of APOE levels in both the brain and peripheral blood in postnatal mice.

Surprisingly, it was discovered that postnatal administration of an APOE receptor agonist (APOE-RA) mitigates motor learning deficits and anxiety in those mice. Remarkably, several molecular and electrophysiological properties essential for learning, which are altered by PAE, are restored by APOE-RA.

A further aspect of the invention is partly based on the discovery, in view of a human genome-wide association study provided herein, that the interaction of PAE and a single nucleotide polymorphism in the APOE enhancer which chromatin is closed by PAE in mice is associated with lower scores in the delayed matching-to-sample task in children. APOE in the plasma is also reduced in PAE children, and the reduced level is associated with their lower cognitive performance.

Overall, these findings show that controlling the APOE level serves as an effective treatment for neurobehavioral deficits in FASD.

Thus, in some aspects, provided herein is a method for treating or alleviating a fetal alcohol spectrum disorder (FASD) (e.g., fetal alcohol syndrome (FAS), partial fetal alcohol syndrome (pFAS), alcohol-related neurodevelopmental disorder (ARND), and neurobehavioral disorder associated with prenatal alcohol exposure (ND-PAE)) in a subject, the method comprising administering to the subject an effective amount of an apolipoprotein E (APOE)-modulating therapeutic (e.g., a retinoid X receptor (RXR) agonist, a liver X receptor (LXR) agonist, and an APOE-mimetic therapeutic), thereby treating or alleviating the FASD.

In some aspects, provided herein is a method for treating or alleviating an FASD in a subject, the method comprising administering to the subject an effective amount of an APOE-mimetic therapeutic (e.g., COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2), thereby treating or alleviating the FASD.

In some aspects, provided herein is a method of preventing or reducing the risk of an FASD in a fetus of a pregnant subject, the method comprising administering to the subject an effective amount of an APOE-modulating therapeutic, thereby preventing or reducing the risk the FASD in the fetus.

In some aspects, provided herein is a method of identifying a subject having an FASD, the method comprising: (a) measuring the level of APOE in a biological sample from the subject, and (b) comparing the measured level of APOE to a control level, wherein a lower level of APOE in the biological sample from the subject compared to the control level is indicative of the subject having the FASD.

This application is based on a published article which was published on May 11, 2024, Hwang et al., Reduction of APOE accounts for neurobehavioral deficits in fetal alcohol spectrum disorders. Mol Psychiatry (2024), which is expressly incorporated by reference in its entirety.

With the general aspects of the invention described above, further detailed embodiments are provided in the sections below.

It should be understood that any embodiments of described herein, including embodiments described only in one section or only in the claims, can be combined with any one or more additional embodiments, unless expressly disclaimed or unless improper.

### Methods

Provided herein are methods for treating, preventing, or alleviating a fetal alcohol spectrum disorder (FASD) in a subject. In some embodiments, the method comprising administering to the subject an effective amount of an apolipoprotein E (APOE)-modulating therapeutic or a pharmaceutical composition thereof, thereby treating, preventing, or alleviating the FASD.

In some embodiments, the APOE-modulating therapeutic is selected from the group consisting of a retinoid X receptor (RXR) agonist, a liver X receptor (LXR) agonist, and an APOE-mimetic therapeutic. In some embodiments, the FASD is selected from the group consisting of fetal alcohol syndrome (FAS), partial fetal alcohol syndrome (pFAS), alcohol-related neurodevelopmental disorder (ARND), and neurobehavioral disorder associated with prenatal alcohol exposure (ND-PAE).

The term “administering” as used herein is meant to refer to the delivery of a substance (e.g., an APOE modulating therapeutic) to achieve a therapeutic objective (e.g., treatment, prevention, or ameliorating symptoms of a disease or condition). Modes of administration may be parenteral, enteral, and topical. Parenteral administration is usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Enteral administration refers to administration into the gastrointestinal tract including, but not limited to, orally (PO), rectally (PR), or through a tube such as a nasogastric (NG) tube, nasointestinal (NI) tube, or percutaneous endoscopic gastrostomy (PEG) tube. Topical administration refers to the application of a substance to a particular place on or in the body. This route is commonly used for delivery to the skin, eyes, nose, vagina, and rectum, and may involve various formulation, such as creams, ointments, gels, lotions, sprays, patches, drops, and foams.

“Treatment” as used herein, refers to therapeutic treatment, for example, wherein the object is to lessen one or more symptoms of the condition or disorder. In certain embodiments, the term “treatment” covers any administration or application of a therapeutic for disease in a patient, and includes inhibiting or slowing the disease or progression of the disease; partially or fully relieving the disease, for example, by causing regression, or restoring or repairing a lost, missing, or defective function; stimulating an inefficient process; or causing the disease plateau to have reduced severity. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

As used herein, the term “alleviating” refers to a lessening or improvement of one or more symptoms associated with the condition or disorder, as compared to not administering an a treatment (e.g., an APOE-modulating therapeutic). “Alleviating” also includes shortening or reduction in duration of a symptom.

As used herein, the term “preventing” refers to prevention and/or delay of the onset of a condition, disorder and/or a symptom(s) in a subject and/or a reduction in the severity of the onset of the condition, disorder and/or symptom(s) relative to what would occur in the absence of the methods of the invention. The prevention can be complete, e.g., the total absence of the condition, disorder and/or clinical symptom(s). The prevention can also be partial, such that the occurrence of the condition, disorder and/or clinical symptom(s) in the subject and/or the severity of onset is less than what would occur in the absence of the present invention.

As used herein, the term “effective amount” or “therapeutically effective amount” refers to the amount of a drug, e.g., an APOE-modulating therapeutic, which is sufficient to reduce or ameliorate the severity and/or duration of a condition or disorder, or one or more symptoms thereof, prevent the advancement of a condition or disorder, cause regression of a condition or disorder, prevent the recurrence, development, onset or progression of one or more symptoms associated with a condition or disorder, detect a condition or disorder, or enhance or improve the prophylactic or therapeutic effect(s) of another therapy (e.g., prophylactic or therapeutic agent). The effective amount of a drug, e.g., an APOE-modulating therapeutic, may, for example, treat or improve or reduce the symptoms associated with a disorder or condition associated with prenatal alcohol exposure (PAE), e.g., FASD.

In some aspects, provided herein is a method of treating, preventing, or alleviating an FASD in a subject, the method comprising administering to the subject an effective amount of an RXR agonist or a pharmaceutical composition thereof, thereby treating, preventing, or alleviating the FASD. In some embodiments, the FASD is selected from the group consisting of FAS, pFAS, ARND, and ND-PAE.

In some aspects, provided herein is a method of treating, preventing, or alleviating an FAS in a subject, the method comprising administering to the subject an effective amount of an RXR agonist or a pharmaceutical composition thereof, thereby treating, preventing, or alleviating the FAS.

In some aspects, provided herein is a method of treating, preventing, or alleviating an ARND in a subject, the method comprising administering to the subject an effective amount of an RXR agonist or a pharmaceutical composition thereof, thereby treating, preventing, or alleviating the ARND.

In some aspects, provided herein is a method of treating, preventing, or alleviating a pFAS in a subject, the method comprising administering to the subject an effective amount of an RXR agonist or a pharmaceutical composition thereof, thereby treating, preventing, or alleviating the pFAS.

In some aspects, provided herein is a method of treating, preventing, or alleviating an ND-PAE in a subject, the method comprising administering to the subject an effective amount of an RXR agonist or a pharmaceutical composition thereof, thereby treating, preventing, or alleviating the ND-PAE.

In some aspects, provided herein is a method of treating, preventing, or alleviating an FASD in a subject, the method comprising administering to the subject an effective amount of an LXR agonist or a pharmaceutical composition thereof, thereby treating, preventing, or alleviating the FASD. In some embodiments, the FASD is selected from the group consisting of FAS, pFAS, ARND, and ND-PAE.

In some aspects, provided herein is a method of treating, preventing, or alleviating an FAS in a subject, the method comprising administering to the subject an effective amount of an LXR agonist or a pharmaceutical composition thereof, thereby treating, preventing, or alleviating the FAS.

In some aspects, provided herein is a method of treating, preventing, or alleviating a pFAS in a subject, the method comprising administering to the subject an effective amount of an LXR agonist or a pharmaceutical composition thereof, thereby treating, preventing, or alleviating the pFAS.

In some aspects, provided herein is a method of treating, preventing, or alleviating an ARND in a subject, the method comprising administering to the subject an effective amount of an LXR agonist or a pharmaceutical composition thereof, thereby treating, preventing, or alleviating the ARND.

In some aspects, provided herein is a method of treating, preventing, or alleviating an ND-PAE in a subject, the method comprising administering to the subject an effective amount of an LXR agonist or a pharmaceutical composition thereof, thereby treating, preventing, or alleviating the ND-PAE.

In some aspects, provided herein is a method for treating, preventing, or alleviating an FASD in a subject, the method comprising administering to the subject an effective amount of an APOE-mimetic therapeutic, thereby treating, preventing, or alleviating the FASD. In some embodiments, the APOE-mimetic therapeutic targets (e.g., binds) to low density lipoprotein receptor-related protein 1 (LRP1), low-density lipoprotein receptor (LDLR), and/or ATP-binding cassette sub-family A member 1 (ABCA1). In some embodiments, the FASD is selected from the group consisting of FAS, pFAS, ARND, and ND-PAE.

In some aspects, provided herein is a method for treating, preventing, or alleviating an FASD in a subject, the method comprising administering to the subject an effective amount of an APOE-mimetic therapeutic, thereby treating, preventing, or alleviating the FASD.

In some embodiments, the APOE-mimetic therapeutic is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2. In some embodiments, the FASD is selected from the group consisting of FAS, pFAS, ARND, and ND-PAE.

In some aspects, provided herein is a method for treating, preventing, or alleviating an FAS in a subject, the method comprising administering to the subject an effective amount of an APOE-mimetic therapeutic, thereby treating, preventing, or alleviating the FAS. In some embodiments, the APOE-mimetic therapeutic targets is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2.

In some aspects, provided herein is a method for treating, preventing, or alleviating an FAS in a subject, the method comprising administering to the subject an effective amount of COG133, thereby treating, preventing, or alleviating the FAS.

In some aspects, provided herein is a method for treating, preventing, or alleviating an FAS in a subject, the method comprising administering to the subject an effective amount of COG1410, thereby treating, preventing, or alleviating the FAS.

In some aspects, provided herein is a method for treating, preventing, or alleviating an FAS in a subject, the method comprising administering to the subject an effective amount of COG112, thereby treating, preventing, or alleviating the FAS.

In some aspects, provided herein is a method for treating, preventing, or alleviating an FAS in a subject, the method comprising administering to the subject an effective amount of CN-105, thereby treating, preventing, or alleviating the FAS.

In some aspects, provided herein is a method for treating, preventing, or alleviating an FAS in a subject, the method comprising administering to the subject an effective amount of Ac-hE18A-NH₂, thereby treating, preventing, or alleviating the FAS.

In some aspects, provided herein is a method for treating, preventing, or alleviating an FAS in a subject, the method comprising administering to the subject an effective amount of AEM-28(R), thereby treating, preventing, or alleviating the FAS.

In some aspects, provided herein is a method for treating, preventing, or alleviating an FAS in a subject, the method comprising administering to the subject an effective amount of AES2-21, thereby treating, preventing, or alleviating the FAS.

In some aspects, provided herein is a method for treating, preventing, or alleviating an FAS in a subject, the method comprising administering to the subject an effective amount of mR18L, thereby treating, preventing, or alleviating the FAS.

In some aspects, provided herein is a method for treating, preventing, or alleviating an FAS in a subject, the method comprising administering to the subject an effective amount of EpK, thereby treating, preventing, or alleviating the FAS.

In some aspects, provided herein is a method for treating, preventing, or alleviating an FAS in a subject, the method comprising administering to the subject an effective amount of hEp, thereby treating, preventing, or alleviating the FAS.

In some aspects, provided herein is a method for treating, preventing, or alleviating an FAS in a subject, the method comprising administering to the subject an effective amount of CS-6253, thereby treating, preventing, or alleviating the FAS.

In some aspects, provided herein is a method for treating, preventing, or alleviating an FAS in a subject, the method comprising administering to the subject an effective amount of ApoE(130-149), thereby treating, preventing, or alleviating the FAS.

In some aspects, provided herein is a method for treating, preventing, or alleviating an FAS in a subject, the method comprising administering to the subject an effective amount of ApoE(141-155)₂, thereby treating, preventing, or alleviating the FAS.

In some aspects, provided herein is a method for treating, preventing, or alleviating an FAS in a subject, the method comprising administering to the subject an effective amount of SP16, thereby treating, preventing, or alleviating the FAS.

In some aspects, provided herein is a method for treating, preventing, or alleviating an FAS in a subject, the method comprising administering to the subject an effective amount of Angiopep-2, thereby treating, preventing, or alleviating the FAS.

In some aspects, provided herein is a method for treating, preventing, or alleviating a pFAS in a subject, the method comprising administering to the subject an effective amount of an APOE-mimetic therapeutic, thereby treating, preventing, or alleviating the pFAS. In some embodiments, the APOE-mimetic therapeutic targets is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2.

In some aspects, provided herein is a method for treating, preventing, or alleviating a pFAS in a subject, the method comprising administering to the subject an effective amount of COG133, thereby treating, preventing, or alleviating the pFAS.

In some aspects, provided herein is a method for treating, preventing, or alleviating a pFAS in a subject, the method comprising administering to the subject an effective amount of COG1410, thereby treating, preventing, or alleviating the pFAS.

In some aspects, provided herein is a method for treating, preventing, or alleviating a pFAS in a subject, the method comprising administering to the subject an effective amount of COG112, thereby treating, preventing, or alleviating the pFAS.

In some aspects, provided herein is a method for treating, preventing, or alleviating a pFAS in a subject, the method comprising administering to the subject an effective amount of CN-105, thereby treating, preventing, or alleviating the pFAS.

In some aspects, provided herein is a method for treating, preventing, or alleviating a pFAS in a subject, the method comprising administering to the subject an effective amount of Ac-hE18A-NH₂, thereby treating, preventing, or alleviating the pFAS.

In some aspects, provided herein is a method for treating, preventing, or alleviating a pFAS in a subject, the method comprising administering to the subject an effective amount of AEM-28(R), thereby treating, preventing, or alleviating the pFAS.

In some aspects, provided herein is a method for treating, preventing, or alleviating a pFAS in a subject, the method comprising administering to the subject an effective amount of AES2-21, thereby treating, preventing, or alleviating the pFAS.

In some aspects, provided herein is a method for treating, preventing, or alleviating a pFAS in a subject, the method comprising administering to the subject an effective amount of mR18L, thereby treating, preventing, or alleviating the pFAS.

In some aspects, provided herein is a method for treating, preventing, or alleviating a pFAS in a subject, the method comprising administering to the subject an effective amount of EpK, thereby treating, preventing, or alleviating the pFAS.

In some aspects, provided herein is a method for treating, preventing, or alleviating a pFAS in a subject, the method comprising administering to the subject an effective amount of hEp, thereby treating, preventing, or alleviating the pFAS.

In some aspects, provided herein is a method for treating, preventing, or alleviating a pFAS in a subject, the method comprising administering to the subject an effective amount of CS-6253, thereby treating, preventing, or alleviating the pFAS.

In some aspects, provided herein is a method for treating, preventing, or alleviating a pFAS in a subject, the method comprising administering to the subject an effective amount of ApoE(130-149), thereby treating, preventing, or alleviating the pFAS.

In some aspects, provided herein is a method for treating, preventing, or alleviating a pFAS in a subject, the method comprising administering to the subject an effective amount of ApoE(141-155)₂, thereby treating, preventing, or alleviating the pFAS.

In some aspects, provided herein is a method for treating, preventing, or alleviating a pFAS in a subject, the method comprising administering to the subject an effective amount of SP16, thereby treating, preventing, or alleviating the pFAS.

In some aspects, provided herein is a method for treating, preventing, or alleviating a pFAS in a subject, the method comprising administering to the subject an effective amount of Angiopep-2, thereby treating, preventing, or alleviating the pFAS.

In some aspects, provided herein is a method for treating, preventing, or alleviating an ARND in a subject, the method comprising administering to the subject an effective amount of an APOE-mimetic therapeutic, thereby treating, preventing, or alleviating the ARND. In some embodiments, the APOE-mimetic therapeutic targets is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2.

In some aspects, provided herein is a method for treating, preventing, or alleviating an ARND in a subject, the method comprising administering to the subject an effective amount of COG133, thereby treating, preventing, or alleviating the ARND.

In some aspects, provided herein is a method for treating, preventing, or alleviating an ARND in a subject, the method comprising administering to the subject an effective amount of COG1410, thereby treating, preventing, or alleviating the ARND.

In some aspects, provided herein is a method for treating, preventing, or alleviating an ARND in a subject, the method comprising administering to the subject an effective amount of COG112, thereby treating, preventing, or alleviating the ARND.

In some aspects, provided herein is a method for treating, preventing, or alleviating an ARND in a subject, the method comprising administering to the subject an effective amount of CN-105, thereby treating, preventing, or alleviating the ARND.

In some aspects, provided herein is a method for treating, preventing, or alleviating an ARND in a subject, the method comprising administering to the subject an effective amount of Ac-hE18A-NH₂, thereby treating, preventing, or alleviating the ARND.

In some aspects, provided herein is a method for treating, preventing, or alleviating an ARND in a subject, the method comprising administering to the subject an effective amount of AEM-28(R), thereby treating, preventing, or alleviating the ARND.

In some aspects, provided herein is a method for treating, preventing, or alleviating an ARND in a subject, the method comprising administering to the subject an effective amount of AES2-21, thereby treating, preventing, or alleviating the ARND.

In some aspects, provided herein is a method for treating, preventing, or alleviating an ARND in a subject, the method comprising administering to the subject an effective amount of mR18L, thereby treating, preventing, or alleviating the ARND.

In some aspects, provided herein is a method for treating, preventing, or alleviating an ARND in a subject, the method comprising administering to the subject an effective amount of EpK, thereby treating, preventing, or alleviating the ARND.

In some aspects, provided herein is a method for treating, preventing, or alleviating an ARND in a subject, the method comprising administering to the subject an effective amount of hEp, thereby treating, preventing, or alleviating the ARND.

In some aspects, provided herein is a method for treating, preventing, or alleviating an ARND in a subject, the method comprising administering to the subject an effective amount of CS-6253, thereby treating, preventing, or alleviating the ARND.

In some aspects, provided herein is a method for treating, preventing, or alleviating an ARND in a subject, the method comprising administering to the subject an effective amount of ApoE(130-149), thereby treating, preventing, or alleviating the ARND.

In some aspects, provided herein is a method for treating, preventing, or alleviating an ARND in a subject, the method comprising administering to the subject an effective amount of ApoE(141-155)₂, thereby treating, preventing, or alleviating the ARND.

In some aspects, provided herein is a method for treating, preventing, or alleviating an ARND in a subject, the method comprising administering to the subject an effective amount of SP16, thereby treating, preventing, or alleviating the ARND.

In some aspects, provided herein is a method for treating, preventing, or alleviating an ARND in a subject, the method comprising administering to the subject an effective amount of Angiopep-2, thereby treating, preventing, or alleviating the ARND.

In some aspects, provided herein is a method for treating, preventing, or alleviating an ND-PAE in a subject, the method comprising administering to the subject an effective amount of an APOE-mimetic therapeutic, thereby treating, preventing, or alleviating the ND-PAE.

In some embodiments, the APOE-mimetic therapeutic targets is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2.

In some aspects, provided herein is a method for treating, preventing, or alleviating an ND-PAE in a subject, the method comprising administering to the subject an effective amount of COG133, thereby treating, preventing, or alleviating the ND-PAE.

In some aspects, provided herein is a method for treating, preventing, or alleviating an ND-PAE in a subject, the method comprising administering to the subject an effective amount of COG1410, thereby treating, preventing, or alleviating the ND-PAE.

In some aspects, provided herein is a method for treating, preventing, or alleviating an ND-PAE in a subject, the method comprising administering to the subject an effective amount of COG112, thereby treating, preventing, or alleviating the ND-PAE.

In some aspects, provided herein is a method for treating, preventing, or alleviating an ND-PAE in a subject, the method comprising administering to the subject an effective amount of CN-105, thereby treating, preventing, or alleviating the ND-PAE.

In some aspects, provided herein is a method for treating, preventing, or alleviating an ND-PAE in a subject, the method comprising administering to the subject an effective amount of Ac-hE18A-NH₂, thereby treating, preventing, or alleviating the ND-PAE.

In some aspects, provided herein is a method for treating, preventing, or alleviating an ND-PAE in a subject, the method comprising administering to the subject an effective amount of AEM-28(R), thereby treating, preventing, or alleviating the ND-PAE.

In some aspects, provided herein is a method for treating, preventing, or alleviating an ND-PAE in a subject, the method comprising administering to the subject an effective amount of AES2-21, thereby treating, preventing, or alleviating the ND-PAE.

In some aspects, provided herein is a method for treating, preventing, or alleviating an ND-PAE in a subject, the method comprising administering to the subject an effective amount of mR18L, thereby treating, preventing, or alleviating the ND-PAE.

In some aspects, provided herein is a method for treating, preventing, or alleviating an ND-PAE in a subject, the method comprising administering to the subject an effective amount of EpK, thereby treating, preventing, or alleviating the ND-PAE.

In some aspects, provided herein is a method for treating, preventing, or alleviating an ND-PAE in a subject, the method comprising administering to the subject an effective amount of hEp, thereby treating, preventing, or alleviating the ND-PAE.

In some aspects, provided herein is a method for treating, preventing, or alleviating an ND-PAE in a subject, the method comprising administering to the subject an effective amount of CS-6253, thereby treating, preventing, or alleviating the ND-PAE.

In some aspects, provided herein is a method for treating, preventing, or alleviating an ND-PAE in a subject, the method comprising administering to the subject an effective amount of ApoE(130-149), thereby treating, preventing, or alleviating the ND-PAE.

In some aspects, provided herein is a method for treating, preventing, or alleviating an ND-PAE in a subject, the method comprising administering to the subject an effective amount of ApoE(141-155)₂, thereby treating, preventing, or alleviating the ND-PAE.

In some aspects, provided herein is a method for treating, preventing, or alleviating an ND-PAE in a subject, the method comprising administering to the subject an effective amount of SP16, thereby treating, preventing, or alleviating the ND-PAE.

In some aspects, provided herein is a method for treating, preventing, or alleviating an ND-PAE in a subject, the method comprising administering to the subject an effective amount of Angiopep-2, thereby treating, preventing, or alleviating the ND-PAE.

In some aspects, provided herein is a method for treating, preventing, or alleviating an FAS in a subject, the method comprising administering to the subject an effective amount of an COG133, thereby treating, preventing, or alleviating the FAS. In some embodiments, COG133 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1. In some embodiments, COG133 comprises an amino acid sequence having at least 90% identity to SEQ ID NO: 1. In some embodiments, COG133 comprises an amino acid sequence having at most 1, 2, 3, 4, 5, 6, 7, 8, 9, and 10 residue changes compared to SEQ ID NO: 1. In some embodiments, COG133 comprises an amino acid sequence having at least about 70%, 75%, 80%, 85%, 90%, 95%, 96%, 97%, 98% or 99% activity (e.g., target binding affinity) compared to that of SEQ ID NO: 1. In some embodiments, COG133 comprises the amino acid sequence of SEQ ID NO: 1.

In some aspects, provided herein is a method for treating, preventing, or alleviating a pFAS in a subject, the method comprising administering to the subject an effective amount of COG133, thereby treating, preventing, or alleviating the pFAS. In some embodiments, COG133 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1. In some embodiments, COG133 comprises an amino acid sequence having at least 90% identity to SEQ ID NO: 1. In some embodiments, COG133 comprises an amino acid sequence having at most 1, 2, 3, 4, 5, 6, 7, 8, 9, and 10 residue changes compared to SEQ ID NO: 1. In some embodiments, COG133 comprises an amino acid sequence having at least about 70%, 75%, 80%, 85%, 90%, 95%, 96%, 97%, 98% or 99% activity (e.g., target binding affinity) compared to that of SEQ ID NO: 1. In some embodiments, COG133 comprises the amino acid sequence of SEQ ID NO: 1.

In some aspects, provided herein is a method for treating, preventing, or alleviating an ARND in a subject, the method comprising administering to the subject an effective amount of COG133, thereby treating, preventing, or alleviating the ARND. In some embodiments, COG133 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1. In some embodiments, COG133 comprises an amino acid sequence having at least 90% identity to SEQ ID NO: 1. In some embodiments, COG133 comprises an amino acid sequence having at most 1, 2, 3, 4, 5, 6, 7, 8, 9, and 10 residue changes compared to SEQ ID NO: 1. In some embodiments, COG133 comprises an amino acid sequence having at least about 70%, 75%, 80%, 85%, 90%, 95%, 96%, 97%, 98% or 99% activity (e.g., target binding affinity) compared to that of SEQ ID NO: 1. In some embodiments, COG133 comprises the amino acid sequence of SEQ ID NO: 1.

In some aspects, provided herein is a method for treating, preventing, or alleviating an ND-PAE in a subject, the method comprising administering to the subject an effective amount of COG133, thereby treating, preventing, or alleviating the ND-PAE. In some embodiments, COG133 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1. In some embodiments, COG133 comprises an amino acid sequence having at least 90% identity to SEQ ID NO: 1. In some embodiments, COG133 comprises an amino acid sequence having at most 1, 2, 3, 4, 5, 6, 7, 8, 9, and 10 residue changes compared to SEQ ID NO: 1. In some embodiments, COG133 comprises an amino acid sequence having at least about 70%, 75%, 80%, 85%, 90%, 95%, 96%, 97%, 98% or 99% activity (e.g., target binding affinity) compared to that of SEQ ID NO: 1. In some embodiments, COG133 comprises the amino acid sequence of SEQ ID NO: 1.

In some embodiments, the methods provided herein lessens, improves, or prevents one or more symptoms in the subject. In some embodiments, the one or more symptoms is selected from the group consisting of abnormal facial features (e.g., small eye openings, low nasal bridge, flat midface, smooth philtrum, and/or thin upper lip), low body weight, short height, small head, sleep and sucking problems as a baby, vision (e.g., myopia) and hearing problems, poor coordination, hyperactive behavior, difficulty with attention and memory, learning disabilities and difficulty in school, speech and language delays, intellectual disability or low IQ, poor reasoning and judgment skills, decreased muscle tone, defects with the heart, kidneys, or bones. In some embodiments, the one or more symptoms is selected from the group consisting of a learning deficit, motor deficit, anxiety, nervousness, depression, poor reasoning and judgement, short attention span, hyperactive behavior, poor memory, and speech delay. In some embodiments, the symptom is a learning deficit and/or motor deficit.

In some embodiments, the methods provided herein lessen or improve a motor deficit (e.g., an impairment that results in reduced coordination or control of voluntary muscle movements) in a subject. In some embodiments, the methods provided herein lessen or improve a motor deficit in a subject by at least at 10%, at least at 20%, at least at 30%, at least at 40%, at least at 50%, at least at 60%, at least at 70%, at least at 80%, at least at 90%, at least at 100%, at least at 110%, at least at 120%, at least 130, or at least 140% as compared to an individual with PAE who was not administered the drug. In some embodiments, the methods provided restore a motor deficit in a subject as compared to an individual without PAE. In some embodiments, the methods provided herein eliminate a motor deficit in a subject. In some embodiments, the methods provided herein prevent a motor deficit in a subject. In some aspects, the method comprises administering to the subject an effective amount of COG133. In some embodiments, COG133 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1. In some embodiments, COG133 comprises an amino acid sequence having at least 90% identity to SEQ ID NO: 1. In some embodiments, COG133 comprises an amino acid sequence having at most 1, 2, 3, 4, 5, 6, 7, 8, 9, and 10 residue changes compared to SEQ ID NO: 1. In some embodiments, COG133 comprises an amino acid sequence having at least about 70%, 75%, 80%, 85%, 90%, 95%, 96%, 97%, 98% or 99% activity (e.g., target binding affinity) compared to that of SEQ ID NO: 1. In some embodiments, COG133 comprises the amino acid sequence of SEQ ID NO: 1.

Any suitable method know in the art can be used to measure a motor deficit (e.g., the Bayley Scales of Infant Development (BSID-II), Psychomotor Development Index (PDI) and Mental Development Index (MDI), coordination tests (e.g., finger-to-nose, heel-to-shin), Movement Assessment Battery for Children—Second Edition (MABC-2), Bruininks-Oseretsky Test of Motor Proficiency, Second Edition (BOT-2), etc.).

In some embodiments, the methods provided herein lessen or improve anxiety in a subject. In some embodiments, the methods provided herein lessen or improve anxiety in a subject by at least at 10%, at least at 20%, at least at 30%, at least at 40%, at least at 50%, at least at 60%, at least at 70%, at least at 80%, at least at 90%, at least at 100%, at least at 110%, at least at 120%, at least 130, or at least 140% as compared to an individual with PAE who was not administered the drug. In some embodiments, the methods provided herein prevent anxiety in a subject. In some embodiments, the methods provided herein eliminate anxiety in a subject. In some aspects, the method comprises administering to the subject an effective amount of COG133. In some embodiments, COG133 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1. In some embodiments, COG133 comprises an amino acid sequence having at least 90% identity to SEQ ID NO: 1. In some embodiments, COG133 comprises an amino acid sequence having at most 1, 2, 3, 4, 5, 6, 7, 8, 9, and 10 residue changes compared to SEQ ID NO: 1. In some embodiments, COG133 comprises an amino acid sequence having at least about 70%, 75%, 80%, 85%, 90%, 95%, 96%, 97%, 98% or 99% activity (e.g., target binding affinity) compared to that of SEQ ID NO: 1. In some embodiments, COG133 comprises the amino acid sequence of SEQ ID NO: 1. Any suitable method know in the art can be used to measure anxiety (e.g. Revised Children's Anxiety and Depression Scale (RCADS), GAD-7 (Generalized Anxiety Disorder-7), Hamilton Anxiety Rating Scale (HAM-A), etc.).

In some aspects, provided herein is a method of preventing or reducing the risk of an FASD in a fetus of a pregnant subject. In some embodiments, the method comprises administering to the subject an effective amount of an APOE-modulating therapeutic (e.g., an RXR agonist, LXR agonist, and APOE-mimetic therapeutic), thereby preventing or reducing the risk the FASD in the fetus. In some embodiments, the pregnant subject has or is suspected of having consumed alcohol while pregnant. In some embodiments, the FASD is selected from the group consisting of FAS, pFAS, ARND, and ND-PAE. In some embodiments, the APOE-modulating therapeutic is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2. In some embodiments, the fetus of the subject carries a mutation in an APOE enhancer element (e.g., a single nucleotide polymorphism (SNP) such as rs584007). In some embodiments, administration of the APOE-modulating therapeutic prevents or reduces the risk of the fetus developing one or more of the following systems after birth: a learning deficit, motor deficit, anxiety, depression, poor reasoning and judgement, short attention span, hyperactive behavior, poor memory, and speech delay.

In some aspects, provided herein is a method of increasing APOE activity in a subject (e.g., lipoprotein-mediated lipid transport; production, conversion and clearance of plasma lipoproteins; binding to cellular receptors including the LDL receptor/LDLR, the LDL receptor-related proteins LRP1, LRP2 and LRP8 and the very low-density lipoprotein receptor/VLDLR that mediate the cellular uptake of the APOE-containing lipoprotein particles; heparin-binding activity; innate and adaptive immune responses; and lipid transport in the central nervous system, regulating neuron survival and sprouting, etc.). In some embodiments, the method comprises administering to the subject an effective amount of an APOE-modulating therapeutic (e.g., an RXR agonist, LXR agonist, and APOE-mimetic therapeutic), thereby increasing APOE activity. In some embodiments, the subject has or is suspected of having PAE. In some embodiments, the subject has an FASD. In some embodiments, the FASD is selected from the group consisting of FAS, pFAS, ARND, and ND-PAE. In some embodiments, the APOE-modulating therapeutic is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2. In some embodiments, the subject carries a mutation in an APOE enhancer element (e.g., a single nucleotide polymorphism (SNP) such as rs584007). In some embodiments, the subject has low levels of plasma APOE as compared to an individual without PAE or an FASD. In some embodiments, increasing APOE activity prevents, treats, or alleviates one or more of the following systems: a learning deficit, motor deficit, anxiety, depression, poor reasoning and judgement, short attention span, hyperactive behavior, poor memory, and speech delay.

An suitable route of administration known in the art can be used to deliver the APOE-modulating therapeutic. In some embodiments, the APOE-modulating therapeutic (e.g., an RXR agonist, LXR agonist, and APOE-mimetic therapeutic) is administered intravenously. In some embodiments, the APOE-modulating therapeutic (e.g., an RXR agonist, LXR agonist, and APOE-mimetic therapeutic) is administered intranasally.

In some embodiments, the APOE-modulating therapeutic (e.g., an RXR agonist, LXR agonist, and APOE-mimetic therapeutic) is administered to the subject postnatally (e.g., after birth). In some embodiments, the APOE-modulating therapeutic (e.g., an RXR agonist, LXR agonist, and APOE-mimetic therapeutic) is administered prenatally (e.g., before birth). In some embodiments, the APOE-modulating therapeutic the administered to a pregnant subject.

In some aspects, provided herein is a method of identifying a subject having PAE or an FASD. In some embodiments, the method comprises: (a) measuring the level of APOE in a biological sample (e.g., plasma sample) from the subject, and (b) comparing the measured level of APOE to a control level. In some embodiments, the control level is the level of APOE in an individual without PAE or an FASD. In some embodiments, a lower level of APOE in the biological sample from the subject compared to the control level is indicative of the subject having the FASD. In some embodiments, the method further comprises selecting or recommending the subject for treatment of FASD with any of the methods of the present disclosure, and optionally treating the subject having FASD with any of the methods of the present disclosure. In some embodiments, the method further comprises administering an APOE-modulating therapeutic (e.g., an RXR agonist, LXR agonist, and APOE-mimetic therapeutic) to the subject if the subject has the FASD, thereby treating the FASD. Suitable techniques for measuring the expression of the level of APOE in a biological sample in vitro (e.g., ELISA, western blotting, mass spectrometry, immunofluorescence, flow cytometry, etc.) will be clear to the skilled person.

### APOE-Modulating Therapeutics

As used herein, an “APOE-modulating therapeutic” refers to a pharmacological agent (e.g., small molecule, biologic, cell therapy, etc.) that enhances and/or increases the expression, function, and/or downstream signaling of APOE to achieve a therapeutic effect (e.g., the treatment, prevention, or alleviation of an FASD).

As used herein, “apolipoprotein E” or “APOE” refers to an apoprotein of the chylomicron. APOE binds to certain liver and peripheral cell receptors, and is plays a role in the catabolism of triglyceride-rich lipoprotein constituents. Exemplary amino acid sequences for APOE can be found in NCBI with Accession Numbers NP_000032.1, NP_001289617.1, NP_001289618.1, NP_001289619.1, and NP_001289620.1. APOE is encoded in humans by the gene APOE. An exemplary nucleic acid sequence for APOE can be found in NCBI with Accession Numbers NM_000041.4, NM_001302688.1, NM_001302689.2, NM_001302690.2, and NM_001302691.2.

In some embodiments, the APOE-modulating therapeutic is selected from the group consisting of a retinoid X receptor (RXR) agonist, a liver X receptor (LXR) agonist, and an APOE-mimetic therapeutic.

In some embodiments, the APOE-modulating therapeutic is an RXR agonist. In some embodiments, the administration of the RXR agonist to the subject increases expression of APOE in the subject. Any suitable RXR agonist known in the art can be used the methods of the present disclosure. In some embodiments, the RXR agonist is bexarotene (4-[1-(5,6,7,8-Tetrahydro-3,5,5,8,8-pentamethyl-2-naphthalenyl)ethenyl]benzoic acid) or a pharmaceutically acceptable salt thereof, CD 3254 (3-[4-Hydroxy-3-(5,6,7,8-tetrahydro-3,5,5,8,8-pentamethyl-2-naphthalenyl)phenyl]-2-propenoic acid) or a pharmaceutically acceptable salt thereof, docosahexaenoic acid ((4Z,7Z,10Z,13Z,16Z,19Z)-4,7,10,13,16,19-Docosahexaenoic acid) or a pharmaceutically acceptable salt thereof, LG 100268 (6-[1-(5,6,7,8-Tetrahydro-3,5,5,8,8-pentamethyl-2-naphthalenyl)cyclopropyl]-3-pyridinecarboxylic acid) or a pharmaceutically acceptable salt thereof, retinoic acid (3,7-Dimethyl-9-(2,6,6-trimethyl-1-cyclohexen-1-yl)-2E,4E,6E,8E,-nonatetraenoic acid) or a pharmaceutically acceptable salt thereof, 9-cis-Retinoic Acid ((2E,4E,6Z,8E)-3,7-dimethyl-9-(2,6,6-trimethylcyclohexen-1-yl)nona-2,4,6,8-tetraenoic acid) or a pharmaceutically acceptable salt thereof, IRX4204 ((2E,4E)-3-methyl-5-((1S,2S)-2-methyl-2-(5,5,8,8-tetramethyl-5,6,7,8-tetrahydronaphthalen-2-yl)cyclopropyl)penta-2,4-dienoic acid) or a pharmaceutically acceptable salt thereof, LG100754 ((2E,4E,6Z)-3-methyl-7-(5,5,8,8-tetramethyl-3-propoxy-5,6,7,8-tetrahydronaphthalen-2-yl)octa-2,4,6-trienoic acid) or a pharmaceutically acceptable salt thereof, or SR 11237 (4-[2-(5,6,7,8-Tetrahydro-5,5,8,8-tetramethyl-2-naphthalenyl)-1,3-dioxolan-2-yl]-benzoic acid) or a pharmaceutically acceptable salt thereof. In some embodiments, the RXR agonist is any RXR agonist disclosed in U.S. Pat. Nos. 9,573,906; 9,908,856; 10,231,947, and 10,328,040 each of which is expressly incorporated by reference in its entirety.

In some embodiments, the APOE-modulating therapeutic is an LXR agonist. In some embodiments, the administration of the LXR agonist to the subject increases expression of APOE in the subject. Any suitable LXR agonist known in the art can be used the methods of the present disclosure. In some embodiments, the LXR agonist is fexaramine (3-[3-[(Cyclohexylcarbonyl)-[[4′-(dimethylamino)-[1,1′-biphenyl]-4-yl]methyl]amino]phenyl]-2-propenoic acid methyl ester) or a pharmaceutically acceptable salt thereof, GW 3965 (3-[3-[[[2-Chloro-3-(trifluoromethyl)phenyl]methyl](2,2-diphenylethyl)amino]propoxy]benzeneacetic acid) or a pharmaceutically acceptable salt thereof, GW 4064 (3-[2-[2-Chloro-4-[[3-(2,6-dichlorophenyl)-5-(1-methylethyl)-4-isoxazolyl]methoxy]phenyl]ethenyl]benzoic acid) or a pharmaceutically acceptable salt thereof, 27-Hydroxycholesterol ((3β,25R)-Cholest-5-ene-3,26-diol) or a pharmaceutically acceptable salt thereof, T 0901317 (N-(2,2,2-Trifluoroethyl)-N-[4-[2,2,2-trifluoro-1-hydroxy-1-(trifluoromethyl)ethyl]phenyl]benzenesulfonamide) or a pharmaceutically acceptable salt thereof, LXR-623 (2-(2-chloro-4-fluorobenzyl)-3-(4-fluorophenyl)-7-(trifluoromethyl)-2H-indazole) or a pharmaceutically acceptable salt thereof, Desmosterol or a pharmaceutically acceptable salt thereof, RGX-104 ((R)-2-(3-(3-((2-chloro-3-(trifluoromethyl)benzyl)(2,2-diphenylethyl)amino)butoxy)phenyl)acetic acid) or a pharmaceutically acceptable salt thereof, T0901317 (N-(4-(1,1,1,3,3,3-hexafluoro-2-hydroxypropan-2-yl)phenyl)-N-(2,2,2-trifluoroethyl)benzenesulfonamide) or a pharmaceutically acceptable salt thereof, or WAY 252623 (2-[(2-Chloro-4-fluorophenyl)methyl]-3-(4-fluorophenyl)-7-(trifluoromethyl)-2H-indazole) or a pharmaceutically acceptable salt thereof. In some embodiments, the LXR agonist is any LXR agonist disclosed in US Patent Publication Nos. 2013/0345220 A1, 2024/0293401 A1, and 2022/0363662 A1 and U.S. Pat. Nos. 8,987,318 and 12,209,180 each of which is expressly incorporated by reference in its entirety.

As used herein, an “APOE-modulating therapeutic” refers to a pharmacological agent (e.g., a peptide) that that replicates the function and/or downstream signaling of APOE to achieve a therapeutic effect (e.g., the treatment, prevention, or alleviation of an FASD).

In some embodiments, the APOE-modulating therapeutic is an APOE-mimetic therapeutic. In some embodiments, mimics the biological activity of endogenous APOE protein. Any suitable APOE-mimetic therapeutic known in the art can be used the methods of the present disclosure. In some embodiments, the APOE-mimetic therapeutic targets (e.g., binds) to low density lipoprotein receptor-related protein 1 (LRP1), low-density lipoprotein receptor (LDLR), and/or ATP-binding cassette sub-family A member 1 (ABCA1).

In some embodiments, the APOE-mimetic therapeutic is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 1. In some embodiments, the APOE-mimetic therapeutic comprises an amino acid sequence having at least 90% identity to SEQ ID NO: 1. In some embodiments, the APOE-mimetic therapeutic comprises an amino acid sequence having at most 1, 2, 3, 4, 5, 6, 7, 8, 9, and 10 residue changes compared to SEQ ID NO: 1. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 1. In some embodiments, the APOE-mimetic therapeutic comprises the amino acid sequence of SEQ ID NO: 1. In some embodiments, the APOE-mimetic therapeutic is COG133.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2. In some embodiments, the APOE-mimetic therapeutic comprises an amino acid sequence having at least 90% identity to SEQ ID NO: 2. In some embodiments, the APOE-mimetic therapeutic comprises an amino acid sequence having at most 1, 2, 3, 4, 5, 6, 7, 8, 9, and 10 residue changes compared to SEQ ID NO: 2. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 2. In some embodiments, the APOE-mimetic therapeutic comprises the amino acid sequence of SEQ ID NO: 2. In some embodiments, the APOE-mimetic therapeutic is COG1410.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 3. In some embodiments, the APOE-mimetic therapeutic comprises an amino acid sequence having at least 90% identity to SEQ ID NO: 3. In some embodiments, the APOE-mimetic therapeutic comprises an amino acid sequence having at most 1, 2, 3, 4, 5, 6, 7, 8, 9, and 10 residue changes compared to SEQ ID NO: 3. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 3. In some embodiments, the APOE-mimetic therapeutic comprises the amino acid sequence of SEQ ID NO: 3. In some embodiments, the APOE-mimetic therapeutic is COG112.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 4. In some embodiments, the APOE-mimetic therapeutic comprises an amino acid sequence having at least 90% identity to SEQ ID NO: 4. In some embodiments, the APOE-mimetic therapeutic comprises an amino acid sequence having at most 1, 2, 3, 4, 5, 6, 7, 8, 9, and 10 residue changes compared to SEQ ID NO: 4. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 4. In some embodiments, the APOE-mimetic therapeutic comprises the amino acid sequence of SEQ ID NO: 4. In some embodiments, the APOE-mimetic therapeutic is CN-105.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 5. In some embodiments, the APOE-mimetic therapeutic comprises an amino acid sequence having at least 90% identity to SEQ ID NO: 5. In some embodiments, the APOE-mimetic therapeutic comprises an amino acid sequence having at most 1, 2, 3, 4, 5, 6, 7, 8, 9, and 10 residue changes compared to SEQ ID NO: 5. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 5. In some embodiments, the APOE-mimetic therapeutic comprises the amino acid sequence of SEQ ID NO: 5. In some embodiments, the APOE-mimetic therapeutic is Ac-hE18A-NH₂.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 6. In some embodiments, the APOE-mimetic therapeutic comprises an amino acid sequence having at least 90% identity to SEQ ID NO: 6. In some embodiments, the APOE-mimetic therapeutic comprises an amino acid sequence having at most 1, 2, 3, 4, 5, 6, 7, 8, 9, and 10 residue changes compared to SEQ ID NO: 6. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 6. In some embodiments, the APOE-mimetic therapeutic comprises the amino acid sequence of SEQ ID NO: 6. In some embodiments, the APOE-mimetic therapeutic is AEM-28(R).

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 7. In some embodiments, the APOE-mimetic therapeutic comprises an amino acid sequence having at least 90% identity to SEQ ID NO: 7. In some embodiments, the APOE-mimetic therapeutic comprises an amino acid sequence having at most 1, 2, 3, 4, 5, 6, 7, 8, 9, and 10 residue changes compared to SEQ ID NO: 7. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 7. In some embodiments, the APOE-mimetic therapeutic comprises the amino acid sequence of SEQ ID NO: 7. In some embodiments, the APOE-mimetic therapeutic is AES2-21.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 8. In some embodiments, the APOE-mimetic therapeutic comprises an amino acid sequence having at least 90% identity to SEQ ID NO: 8. In some embodiments, the APOE-mimetic therapeutic comprises an amino acid sequence having at most 1, 2, 3, 4, 5, 6, 7, 8, 9, and 10 residue changes compared to SEQ ID NO: 8. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 8. In some embodiments, the APOE-mimetic therapeutic comprises the amino acid sequence of SEQ ID NO: 8. In some embodiments, the APOE-mimetic therapeutic is mR18L.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 9. In some embodiments, the APOE-mimetic therapeutic comprises an amino acid sequence having at least 90% identity to SEQ ID NO: 9. In some embodiments, the APOE-mimetic therapeutic comprises an amino acid sequence having at most 1, 2, 3, 4, 5, 6, 7, 8, 9, and 10 residue changes compared to SEQ ID NO: 9. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 9. In some embodiments, the APOE-mimetic therapeutic comprises the amino acid sequence of SEQ ID NO: 9. In some embodiments, the APOE-mimetic therapeutic is EpK.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 10. In some embodiments, the APOE-mimetic therapeutic comprises an amino acid sequence having at least 90% identity to SEQ ID NO: 10. In some embodiments, the APOE-mimetic therapeutic comprises an amino acid sequence having at most 1, 2, 3, 4, 5, 6, 7, 8, 9, and 10 residue changes compared to SEQ ID NO: 10. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 10. In some embodiments, the APOE-mimetic therapeutic comprises the amino acid sequence of SEQ ID NO: 10. In some embodiments, the APOE-mimetic therapeutic is hEp.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 11. In some embodiments, the APOE-mimetic therapeutic comprises an amino acid sequence having at least 90% identity to SEQ ID NO: 11. In some embodiments, the APOE-mimetic therapeutic comprises an amino acid sequence having at most 1, 2, 3, 4, 5, 6, 7, 8, 9, and 10 residue changes compared to SEQ ID NO: 11. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 11. In some embodiments, the APOE-mimetic therapeutic comprises the amino acid sequence of SEQ ID NO: 11. In some embodiments, the APOE-mimetic therapeutic is CS-6253.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 12. In some embodiments, the APOE-mimetic therapeutic comprises an amino acid sequence having at least 90% identity to SEQ ID NO: 12. In some embodiments, the APOE-mimetic therapeutic comprises an amino acid sequence having at most 1, 2, 3, 4, 5, 6, 7, 8, 9, and 10 residue changes compared to SEQ ID NO: 12. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 12. In some embodiments, the APOE-mimetic therapeutic comprises the amino acid sequence of SEQ ID NO: 12. In some embodiments, the APOE-mimetic therapeutic is ApoE(130-149).

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 13. In some embodiments, the APOE-mimetic therapeutic comprises an amino acid sequence having at least 90% identity to SEQ ID NO: 13. In some embodiments, the APOE-mimetic therapeutic comprises an amino acid sequence having at most 1, 2, 3, 4, 5, 6, 7, 8, 9, and 10 residue changes compared to SEQ ID NO: 13. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 13. In some embodiments, the APOE-mimetic therapeutic comprises the amino acid sequence of SEQ ID NO: 13. In some embodiments, the APOE-mimetic therapeutic is ApoE(141-155)₂.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 14. In some embodiments, the APOE-mimetic therapeutic comprises an amino acid sequence having at least 90% identity to SEQ ID NO: 14. In some embodiments, the APOE-mimetic therapeutic comprises an amino acid sequence having at most 1, 2, 3, 4, 5, 6, 7, 8, 9, and 10 residue changes compared to SEQ ID NO: 14. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 14. In some embodiments, the APOE-mimetic therapeutic comprises the amino acid sequence of SEQ ID NO: 14. In some embodiments, the APOE-mimetic therapeutic is SP16.

In some embodiments, the APOE-mimetic therapeutic comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 15. In some embodiments, the APOE-mimetic therapeutic comprises an amino acid sequence having at least 90% identity to SEQ ID NO: 15. In some embodiments, the APOE-mimetic therapeutic comprises an amino acid sequence having at most 1, 2, 3, 4, 5, 6, 7, 8, 9, and 10 residue changes compared to SEQ ID NO: 15. In some embodiments, the APOE-mimetic therapeutic comprises an amino acid sequence having at least about 70%, 75%, 80%, 85%, 90%, 95%, 96%, 97%, 98% or 99% activity (e.g., target binding affinity) compared to that of SEQ ID NO: 15. In some embodiments, the APOE-mimetic therapeutic comprises the amino acid sequence of SEQ ID NO: 15. In some embodiments, the APOE-mimetic therapeutic is Angiopep-2.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In some embodiments, the APOE-mimetic therapeutic is an APOE-mimetic therapeutic set forth in.

TABLE 1
APOE-mimetic therapeutics

| Peptide | | | Primary | Key Indication / |
|---|---|---|---|---|
| Name | Amino Acid Sequence | Modifications | Target(s) | Activity |
| COG133 | Ac- | N-terminal acetylation; C- | LRP1, LDLR; | Reduces |
| | LRVRLASHLRKLRKRL | terminal amidation (17 aa | anti- | neuroinflammation, |
| | L-NH₂ | fragment from ApoE 133- | inflammatory | protects in CNS |
| | (SEQ ID NO: 1) | 149) | | injury models |
| COG1410 | Ac-AS-(Aib)-LRKL- | N-terminal acetylation; C- | LRP1 | Neuroprotection, |
| | (Aib)-KRLL-NH₂ | terminal amidation; | | TBI/stroke models |
| | (SEQ ID NO: 2) | contains a-aminoisobutyric | | |
| | | acid (Aib) substitutions at | | |
| | | two positions (peptide is 12 | | |
| | | aa derived from ApoE | | |
| | | 138-149) | | |
| COG112 | Ac- | N-terminal acetylation; C- | LRP1 | BBB-penetrant, |
| | RQIKIWFQNRRMKWKK | terminal amidation; | | suppresses NF-KB |
| | CLRVRLASHLRKLRKR | chimeric peptide | | inflammation |
| | LL-NH₂ | (Antennapedia/penetratin | | |
| | (SEQ ID NO: 3) | sequence + Cys + ApoE | | |
| | | 133-149 fragment) | | |
| CN-105 | Ac-VSRRR-NH₂ | N-terminal acetylation; C- | LRP1 | BBB-permeable, |
| | (SEQ ID NO: 4) | terminal amidation; | | Phase 1 tested in |
| | | pentapeptide (derived from | | humans (ICH, SAH) |
| | | ApoE receptor-binding | | |
| | | region) | | |
| Ac- | Ac- | N-terminal acetylation; C- | LRP1, LDLR; | Anti-atherogenic, |
| hE18A- | LRKLRKRLLRDWLKAF | terminal amidation; 28 aa | ABCA1 | lipid lowering, |
| NH₂ | YDKVAEKLKEAF-NH₂ | dual-domain peptide | | Phase 1 safety |
| (AEM-28) | (SEQ ID NO: 5) | (ApoE(141-150) + 18A | | |
| | | amphipathic helix) | | |
| AEM- | Ac-LRRLRRRLLR- | N-terminal acetylation; C- | ABCA1, LRP1 | Enhanced potency, |
| 28(R) | DWLKAFYDKVAEKLK | terminal amidation; Arg- | | preclinical |
| | EAF-NH₂ | rich variant of Ac-hE18A | | |
| | (SEQ ID NO: 6) | (all Lys→Arg in the ApoE | | |
| | | segment) | | |
| AES2-21 | Ac-Ahx-LRRLRRRLLR- | N-terminal acetylation; C- | ABCA1 | Next-gen variant, |
| | DWLKAFYDKVAEKLK | terminal amidation; | | stable activity |
| | EAF-NH₂ | includes an N-terminal 6- | | |
| | (SEQ ID NO: 7) | aminohexanoic acid spacer | | |
| | | (“Ahx”) attached to Arg- | | |
| | | rich hE18A sequence | | |
| mR18L | Ac- | N-terminal acetylation; C- | ABCA1, LRP1 | Anti-atherogenic, |
| | GFRRFLGSWARIYRAF | terminal amidation; 18 aa | | compared to AEM- |
| | VG-NH₂ | single-domain peptide | | 28 |
| | (SEQ ID NO: 8) | (Arg-modified variant of | | |
| | | an ApoE-mimetic helix) | | |
| EpK | CLRKLRKRLLRKKKKK | Free N-terminal Cys; free | LDLR, | Promotes |
| | KQVAEVRAKLEEQAQQ | C-terminus (38 aa fusion of | ABCA1 | cholesterol efflux, |
| | IRLQAE | ApoE residues 141-150 + | | reduces plaque |
| | (SEQ ID NO: 9) | (KK)_3 linker + 234-254) | | |
| hEp | EELRVRLASHLRKLRK | No terminal modifications | LRP1, LDLR | Anti-atherogenic, |
| | RLLRDADDLQKRLAVY | reported; 61 aa peptide | | reduces |
| | EEQAQQIRLQAEAFQA | linking ApoE residues | | atherosclerosis in |
| | RLKSWFEPL VEDM | 131-162 and 244-272 | | vivo |
| | (SEQ ID NO: 10) | (extended dual-domain) | | |
| CS-6253 | EV(Cit)SKLEEWLAAL(C | Two arginines replaced by | ABCA1 (ApoE | Raises ApoE levels, |
| | it)ELAEELLARLKS | citrulline (Cit); free N- | lipidation | reduces amyloid in |
| | (SEQ ID NO: 11) | terminus and C-terminus | enhancer) | AD models |
| | | (26 aa C-terminal ApoE- | | |
| | | mimetic with Cit | | |
| | | substitutions) | | |
| ApoE | CGRLVQYRGEVQAML | No modifications; 20 aa | LRP1, LDLR | High-affinity |
| (130-149) | GQSTE | native apoE receptor- | | binding (~100 nM) |
| | (SEQ ID NO: 12) | binding segment (residues | | |
| | | 130-149) | | |
| ApoE | QAMLGQSTEELRVRL | Typically used as a | LRP1, LDLR | Potent LDLR |
| (141-155)₂ | (homodimer) | disulfide-linked dimer of | | binding |
| | (SEQ ID NO: 13) | two identical 15-aa chains | | |
| | | (each corresponds to apoE | | |
| | | 141-155) | | |
| SP16 | VKFNKPFVFLMIEQNTK | Free termini (synthesized | LRP1 | Cardioprotection; |
| | (SEQ ID NO: 14) | 17 aa peptide from C- | | completed Phase 1 |
| | | terminus of human di- | | |
| | | antitrypsin; LRP1 agonist; | | |
| | | enhanced variant SP163M | | |
| | | is acetylated/amidated with | | |
| | | Met→Nle) | | |
| Angiopep- | TFFYGGSRGKRNNFKT | No modifications (19 aa | LRP1 | Drug delivery |
| 2 | EEY | peptide derived from | | across BBB (e.g. |
| | (SEQ ID NO: 15) | Kunitz domain; brain- | | ANG1005 trials) |
| | | penetrant via LRP1) | | |

In some embodiments, the APOE-mimetic therapeutic is an APOE analog disclosed in U.S. Pat. Nos. 8,288,335 and 7,947,645 each of which is expressly incorporated by reference in its entirety.

In some embodiments, the methods of the present disclosure comprise administering to the subject an effective amount of a pharmaceutical composition comprising the APOE-modulating therapeutic. The compositions may further comprise one or more excipients, carriers, and reagents. The pharmaceutical compositions may comprise the APOE-modulating therapeutic and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition may be formulated to be compatible with its intended route of administration. Examples of routes of administration include parenteral, e.g., intravenous, intranasal, intradermal, subcutaneous, oral (e.g., inhalation), transdermal (e.g., topical), transmucosal, and rectal administration. Solutions or suspensions used for parenteral, intradermal, or subcutaneous application may include one or more of the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation can be enclosed in ampoules, disposable syringes or multiple dose vials made of glass or plastic. In some embodiments, any of the APOE-modulating therapeutics described herein are prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collage, polyorthoesters, and polylactic acid. Methods for preparation of such pharmaceutical compositions and formulations are apparent to those skilled in the art. For example, the APOE-modulating therapeutics may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations may be prepared. Suitable examples of sustained-release preparations include semipermeable matrices of solid hydrophobic polymers containing the APOE-modulating therapeutic, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers (e.g., injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, pharmaceutical compositions suitable for injectable use include sterile aqueous solutions (where water soluble) or dispersions and sterile powders for the extemporaneous preparation of sterile injectable solutions or dispersion. For intravenous administration (e.g., in a syringe or intravenous bag), suitable carriers include physiological saline, bacteriostatic water, Cremophor or phosphate buffered saline (PBS). The composition may be sterile and should be fluid to the extent that easy syringe ability exists. It may be stable under the conditions of manufacture and storage and preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, isotonic agents, for example, sugars, polyalcohols such as mannitol, sorbitol, and sodium chloride may be included in the composition. Prolonged absorption of the injectable compositions may be brought about by including in the composition an agent that delays absorption, for example, aluminum monostearate and gelatin.

In some embodiments, the pharmaceutical composition may comprise a sterile injectable solution. Sterile injectable solutions may be prepared by incorporating the active compound in the required amount in an appropriate solvent with one or a combination of ingredients enumerated above, as required, followed by filtered sterilization. Generally, dispersions may be prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the pharmaceutical composition may be formulized for systemic administration. For example, systemic administration may be by intravenous, intradermal, intraperitoneal, intramuscular, or subcutaneously as well by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds may be formulated into ointments, salves, gels, or creams as generally known in the art.

In some embodiments, the pharmaceutical composition may be prepared with carriers that protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure may be dictated by and directly dependent on the unique characteristics of the active compound and the particular therapeutic effect to be achieved, and the limitations inherent in the art of compounding such an active compound for the treatment of individuals.

In some embodiments, the compositions (e.g., pharmaceutical compositions) may be included in a container, vial, syringe, injector pen, pack, or dispenser, optionally together with instructions for administration.

### Fetal Alcohol Spectrum Disorder (FASD)

As used herein, the term “fetal alcohol spectrum disorder” or “FASD” refers to a range of physical, cognitive, behavioral, and neurodevelopmental impairments that may occur in individuals whose mothers consumed alcohol during pregnancy. FASD can encompass specific diagnoses, including fetal alcohol syndrome (FAS), partial fetal alcohol syndrome (pFAS), alcohol-related neurodevelopmental disorder (ARND), alcohol-related birth defects (ARBD), and neurobehavioral disorder associated with prenatal alcohol exposure (ND-PAE). Diagnosis and classification of FASD are guided by multiple systems known in the art, each employing distinct criteria that generally assess four domains: the level of prenatal alcohol exposure (PAE), growth impairment, dysmorphic facial features, and neurodevelopmental abnormalities. Commonly used diagnostic systems include the FASD 4-Digit Diagnostic Code, the Institute of Medicine (IOM) criteria—revised by Hoyme, the Canadian Guidelines, and the Centers for Disease Control (CDC) guidelines.

In some embodiments, the subject with FASD exhibits one or more symptoms selected from the group consisting of abnormal facial features (e.g., small eye openings, low nasal bridge, flat midface, smooth philtrum, and/or thin upper lip), low body weight, short height, small head, sleep and sucking problems as a baby, vision (e.g., myopia) and hearing problems, poor coordination, hyperactive behavior, difficulty with attention and memory, learning disabilities and difficulty in school, speech and language delays, intellectual disability or low IQ, poor reasoning and judgment skills, decreased muscle tone, defects with the heart, kidneys, or bones. In some embodiments, the subject exhibits one or more symptoms selected from the group consisting of a learning deficit, motor deficit, anxiety, nervousness, depression, poor reasoning and judgement, short attention span, hyperactive behavior, poor memory, and speech delay.

In some embodiments, the FASD is selected from the group consisting of fetal alcohol syndrome (FAS), partial fetal alcohol syndrome (pFAS), alcohol-related neurodevelopmental disorder (ARND), neurobehavioral disorder associated with prenatal alcohol exposure (ND-PAE), and any combination thereof. A person of ordinary skill in the art would understand the clinical features of FAS, ARND, pFAS, and ND-PAE and the criteria for diagnosis.

In some embodiments, the FASD is FAS. In some embodiments, FAS is a clinical diagnosis representing a severe manifestation within the spectrum of disorders caused by prenatal alcohol exposure. In some embodiments, the FAS is characterized by physical, neurological, and behavioral abnormalities. These abnormalities typically include, but are not limited to, low body weight, short height, small head, abnormal facial features (e.g., a smooth ridge between the nose and upper lip, a thin upper lip, and small eyes), vision and/or hearing problems, poor coordination, hyperactive behavior, difficulty with attention and memory, learning disabilities and difficulty in school, speech and language delays, intellectual disability or low IQ, poor reasoning and judgment skills, sleep and sucking problems as a baby, and defects with the heart, kidneys, or bones.

In some embodiments, the FASD is pFAS. In some embodiments, pFAS is a clinical diagnosis that describes a subject who exhibits some of the diagnostic criteria of FAS without all of the features (e.g., growth impairment, decreased head circumference).

In some embodiments, the FASD is ARND. In some embodiments, ARND is a clinical diagnosis that describes a subject with neurodevelopmental and neurobehavioral abnormalities caused by prenatal alcohol exposure. In some embodiments, ARND is characterized by impairments in areas including, but not limited to, cognition, attention, executive functioning, learning, and behavior. In some embodiments, the subject exhibits one or more symptoms selected from the group consisting of hyperactive behavior, difficulty with attention and memory, learning disabilities and difficulty in school, speech and language delays, intellectual disability or low IQ, poor reasoning and judgment skills, and sleep and sucking problems as a baby. In some embodiments, the subject does not exhibit facial dysmorphia or growth deficits.

In some embodiments, the FASD is ND-PAE. In some embodiments, the ND-PAE is a clinical diagnosis that describes a subject with neurodevelopmental and neurobehavioral abnormalities caused by prenatal alcohol exposure. In some embodiments, ARND is characterized by impairments in areas including, but not limited to, neurocognition, self-regulation, and adaptive functioning. In some embodiments, the subject exhibits one or more symptoms selected from the group consisting of hyperactive behavior, difficulty with attention and memory, learning disabilities and difficulty in school, speech and language delays, intellectual disability or low IQ, poor reasoning and judgment skills, and sleep and sucking problems as a baby. In some embodiments, the subject does exhibit facial dysmorphia and/or growth deficits. In some embodiments, the subject does not exhibit facial dysmorphia and/or growth deficits.

In some embodiments, the subject exhibits alcohol-related birth defects (ARBD). In some embodiments, ARBD refers to congenital anomalies resulting from prenatal alcohol exposure. In some embodiments, the subject exhibits abnormalities in organ systems such as the heart, kidneys, bones, and sensory organs, including hearing and vision.

In some embodiments, the subject carries a mutation in an APOE enhancer element. In some embodiments, the mutation in the APOE enhancer element is associated with increased cognitive, behavioral, and/or neurodevelopmental impairments a subject with PAE as compared to individuals with the same genotype but without PAE. In some embodiments, the mutation in the APOE enhancer element increases a subject's risk of an FASD if the subject has PAE. In some embodiments, the mutation is a single nucleotide polymorphism (SNP). In some embodiments, the SNP is rs584007. In some embodiments, the nucleic acid sequence of rs584007 is AGGAGGGGCGTCAGAGGGTGAATAARAGCAGATAGAGTGTTTGGGGGAGGT (SEQ ID NO: 16). Any suitable method known in the art can be used to determine if a subject carries a mutation an APOE enhancer element, including but not limited to, PCR-based techniques (e.g., allele-specific PCR, restriction fragment length polymorphism (RFLP) analysis, TaqMan PCR analysis, high-resolution melting curve analysis, kompetitive allele-specific PCR (KASP), and digital PCR), microarray-based techniques (e.g., SNP microarray), and sequencing-based genotyping (e.g., Sanger or targeted sequencing and next generation sequencing (NGS)-based genotyping).

In some embodiments, the subject has low APOE plasma levels. In some embodiments, the subject has low APOE plasma levels, as compared to APOE plasma levels of an individual without prenatal alcohol exposure (PAE). In some embodiments, the subject has at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, or at least 25% lower APOE plasma levels, as compared to APOE plasma levels of an individual without PAE. In some embodiments, the subject has low APOE plasma levels, as compared to APOE plasma levels of an individual without an FASD. In some embodiments, the subject has at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, or at least 25% lower APOE plasma levels, as compared to APOE plasma levels of an individual without an FASD. In some embodiments, low APOE plasma levels is associated with increased cognitive, behavioral, and/or neurodevelopmental impairments a subject with PAE. Any suitable method known in the art can be used to measure APOE plasma levels in a subject, including but not limited to, ELISA, western blotting, mass spectrometry, immunofluorescence, and flow cytometry.

The terms “subject,” “patient,” and “individual” are used interchangeably herein to refer to a human. In some embodiments, the subject is a female human. In other embodiments, the subject is a male human. In one embodiment, the subject is a pediatric subject. In some embodiments, the subject is an infant. In another embodiment, the subject is an adult subject. In some embodiments, the subject has PAE. In some embodiments, the subject has FASD.

In order that the present disclosure may be better understood, the following examples are set forth. These examples are for purposes of illustration only and are not to be construed as limiting the scope of the present disclosure in any manner.

### EXAMPLES

### Example 1: Reduction of Apoe Accounts for Neurobehavioral Deficits in Fetal Alcohol Spectrum Disorders

In this Example, an epigenetic mechanism that drives the reduction in brain apolipoprotein E (APOE) in PAE mice was identified. The data herein also showed that postnatal administration of an APOE receptor agonist (APOE-RA) rescued both learning deficits and anxiety in prenatal alcohol exposure (PAE) mice. In humans, it was found that a single nucleotide polymorphism (SNP) within an APOE enhancer, in the presence of PAE, is associated with decreased cognitive measures compared to individuals with the same genotype without PAE. Finally, APOE level was reduced in the plasma of PAE children, correlated with their lower cognitive performance. These results indicate that reduced APOE expression due to PAE critically contributes to neurocognitive deficits, and a functional APOE polymorphism is a genetic risk factor that augments the effects of PAE on cognitive performance. Improvement of learning deficits when APOE was increased via an agonist in PAE mice shows that APOE replenishment is a therapy for FASD patients.

### Materials and Methods

### Animal Model

Timed pregnant CD-1 mice were purchased from Charles River Laboratories and maintained on a light-dark cycle (lights on 6:00-18:00) at a constant temperature (22±1° C.). Pregnant mice were randomly assigned to experimental groups. To generate PAE mice (Mohammad S et al. Nat Neurosci. 2020; 23:533-43; Hwang H M et al. Transl Psychiatry. 2022; 12:24), a single dose of 4 g/kg body weight ethanol was administered to pregnant mice via intraperitoneal (i.p.) injection at E16 and E17. Same amount of PBS was administered to generate control mice. Experiments were conducted at around P30, unless otherwise noted. All studies employed a mixture of males and females, and no differences between sexes were observed. All protocols were approved by the Institutional Animal Care and Use Committee (IACUC) of Children's National Hospital. The number of animals and statistical parameters are denoted in the brief description of the drawings.

### Accelerated Rotarod Test

An accelerated rotarod test was performed as described previously (Mohammad et al. Nat Neurosci. 2020; 23:533-43). Briefly, each mouse was placed on a rotating bar, and the time the mouse could maintain balance while the rotation is accelerated to the maximum speed (80 RPM) in 5 min was measured. The testing phase consisted of 2 consecutive days of three trials per day. Each trial was at least 15 min apart and was terminated when the mouse fell off, made one complete rotation without walking on the rotating rod, or reached the maximum speed after the 5-min session. The latency to fall from the rotating rod was scored by automatic timers and falling sensors on the rotarod. Learning index was calculated by averaging the changes in terminal speed of an individual mouse between two consecutive trials.

### Elevated Plus Maze Test

The maze is a grey plus-shaped apparatus with two open arms and two closed arms linked by a central platform. Mice were individually put in the center of the maze facing an open arm and allowed to explore the maze for 5 min. A video was recorded during the experiment, and the time spent in open and closed arms was measured and analyzed with MoutBeat ImageJ Plugin as previously done (Hwang H M et al. Transl Psychiatry. 2022; 12:24).

### Isolation of Peripheral Blood Mononuclear Cells

Mice were deeply anesthetized with isoflurane (Henry Schein, NY, US). Blood was then drawn from their heart via cardiac puncture, immediately transferred to collection tubes containing EDTA-2K, and centrifuged at 1500 RPM for 5 min at room temperature. The top layer, containing primarily blood plasma, was discarded. The remnant, containing peripheral blood mononuclear cells (PBMCs), was mixed with 5 ml of 10% FBS in DMEM/F12 (cat #11320033, Thermo Fisher Scientific, MA, US) and Ficoll-Paque PLUS solution (cat #GE17-1440-02, Millipore Sigma, MA, US) and centrifuged at 1000×g for 15 min at room temperature. The middle layer containing PBMCs was collected, washed twice with PBS, and then transferred to ice-cold FACS (Fluorescence-activated cell sorting) buffer for FACS analysis.

### Fluorescence-Activated Cell Sorting (FACS)

PBMCs were stained with rat monoclonal anti-mouse CD11b antibody (FITC conjugated, cat #101205, Biolegend, CA, US), CD19 antibody (PE-conjugated, cat #152407, Biolegend), and CD90.2 antibody (APC conjugated, cat #105311, Biolegend). Cells were first gated based on cell size [forward scatter (FSC) versus side scatter (SSC)] and singlets (FSC versus trigger pulse width). Then T cells, B cells, and monocytes were separated from each other by collecting the CD11b+/CD19−, CD19+/CD90.2−, and CD19−/CD90.2+ subpopulations of cells, respectively.

### Library Preparation and RNA Sequencing

The cDNA libraries were prepared using the SMART-Seq v4 Ultra Low Input RNA Kit for Sequencing (cat #634888, Takara Bio, Kusatsu, Japan) and Nextera XT DNA Library Prep Kit (cat #FC-131-1096, Illumina, CA, US) as per manufacturer instructions. The unique barcode sequences were incorporated in the adaptors for multiplexed high-throughput sequencing. The final product was assessed for its size distribution and concentration using Bioanalyzer High Sensitivity DNA Kit (cat #5067-4626, Agilent, CA, USA). The libraries were pooled and diluted to 3 nM with 10 mM Tris-HCl, pH 8.5, then denatured using the Illumina protocol. According to manufacturer instructions, the denatured libraries were loaded onto an S1 flow cell on an Illumina NovaSeq 6000 (Illumina) and ran for 2×50 cycles. De-multiplexed sequencing reads were generated using Illumina bcl2fastq (released version 2.18.0.12), allowing no mismatches in the index read.

### RNAA-Seq Data Analysis of PBMC Transcriptome

Low quality reads from the RNA-seq dataset were removed via FastQC (sickle with default setting; available at bioinformatics.babraham.ac.uk/projects/fastqc/). A HISAT2 index was built for the mm10 genome assembly using HISAT2 v2.1.0 (Kim D et al. Nat Biotechnol. 2019; 37:907-15). RNA-sequencing reads of each sample were mapped using HISAT2 or STAR v2.5.4a supplied (Dobin A et al. Bioinformatics. 2013; 29:15-21) with Ensembl annotation file; GRCm38.78.gtf. For the count call, HTseq-count v0.10.0 (Anders S et al. Bioinformatics. 2015; 31:166-9) or featurecounts (Liao Y et al. Bioinformatics. 2014; 30:923-30) was used. Differential gene expression analysis of count data was performed using IRIS Web server-based (bmbl.sdstate.edu/IRIS/) limma-voom v3.38.3 (Ritchie M E et al. Nucleic Acids Res. 2015; 43:e47-e47). First, raw counts were filtered by a cutoff value of count data row sums less than 10 and transformed to log 2 (n+pseudocount). Then, limma-voom was carried out with a minimum fold change of 2 and an adjusted p-value cutoff of 0.05. A volcano plot was generated with the Galaxy platform (usegalaxy.eu/). Quantile normalized counts of differentially expressed genes were clustered by hierarchical k-means clustering using Morpheus (software.broadinstitute.org/morpheus/). The optimal number of clusters was determined by the within sum scale (WSS) method with “wssplot” function in R. Gene ontology enrichment analysis was performed suing Enrichr. The combined scores were generated by taking the log of the p-value from the Fisher exact test and multiplied that by the z-score of the deviation from the expected rank by Enrichr. For Ingenuity Pathway Analysis (IPA; Qiagen, Venlo, Netherlands), cutoff values of expression log ratio of ±1.5 and FDR<0.05 or 0.025 were used to limit the number of genes to be less than 2000 as recommended per the company.

### Quantification of Plasma APOE and Corticosterone in Mice

Whole blood was collected in an EDTA-treated tube (cat #365974, BD, NJ, USA) by cardiac puncture with a 23-25 G needle from a deeply anesthetized animal. The plasma was separated from blood cells by centrifuging the blood sample at 2000×g at 4° C. for 10 min and transferring the supernatant to a clean centrifuge tube. Collected plasma samples were stored in a −80° C. freezer until further analysis. Measurements of APOE and corticosterone were carried out using ELISA Pro: Mouse apoE (cat #3752-1HP-1, Mabtech, Nacka Strand, Sweden) and Mouse Corticosterone ELISA Kit (cat #80556, Crystal Chem, IL, USA), respectively following manufacturer's protocols.

### Free-Floating Immunohistochemistry

Mice were deeply anesthetized with isoflurane (Henry Schein) and perfused transcardially with 10 ml of ice-cold PBS followed by 10 ml of chilled 4% paraformaldehyde (PFA). The brains were removed and post-fixed in the same fixative (4% PFA) at 4° C. overnight. The brains were then incubated in 10% and 30% sucrose solution prepared in PBS for 24 h sequentially at 4° C. After embedding in OCT, coronal sections were cut at 20 or 50 μm on a cryostat (CM3050S, Leica, Wetzlar, Germany).

Immunohistochemistry was performed on free-floating brain sections as follows: Antigen retrieval was performed when necessary following the manufacturer's protocol (cat #00-4955-58, Thermo Fisher Scientific). Then sections were incubated in 30% hydrogen peroxide in methanol (1:4) solution for 30 min at −20° C. to inactivate endogenous peroxidase activity. After rinsing with PBS-T (0.1% Tween-20 in PBS), sections were incubated with 2% BSA for 30 minutes at room temperature for blocking. Sections were then incubated with a primary antibody for APOE (1:500, cat #ab1906, Abcam, Cambridge, UK), NeuN (1:500, cat #ab104225, Abcam), GFAP (1:500, cat #ab4674, Abcam), GluN1 (1:200, cat #32-0500, Thermo Fisher Scientific), LRP1 (1:200, cat #ab92544, Abcam), PSD-95 (1:50, cat #MAI-045, Thermo Fisher Scientific) or KCNN2 (1:500, cat #HPA038221, Sigma-Aldrich, MO, USA) overnight at 4° C. The next day, after washing with PBS-T, sections were incubated with a secondary antibody: horseradish peroxidase (HRP)-conjugated anti-mouse IgG (cat #111-035-146, Jackson ImmunoResearch, PA, US) or anti-rabbit IgG (cat #111-035-144, Jackson ImmunoResearch), or biotinylated anti-rabbit IgG (cat #711-065-152, Jackson ImmunoResearch), diluted at 1:300 for 2 h at room temperature. After washing with PBS-T, sections incubated in the HRP-conjugated secondary antibody were incubated in TSA solution (1:500 diluted in TSA diluent) for 1 h at room temperature. Sections incubated with the biotinylated secondary antibody were incubated in avidin-biotin complex (ABC; cat #32020, Thermo Fisher Scientific) solution for 1 h, followed by 1-h incubation in TSA solution (1:500 diluted in TSA diluent) at room temperature. The following TSA fluorophores were used: TSA plus Fluorescein (cat #NEL741001KT, Akoya Biosciences, MA, USA), Cyanine-3 (cat #NEL744001KT, Akoya Biosciences), Cyanine-5 (cat #NEL745001KT, Akoya Biosciences). For NeuN and GFAP staining, Alexa Fluor 488-conjugated secondary antibody (cat #703-545-155, Jackson ImmunoResearch) was used at 1:300 dilution for 1 h incubation. After washing with PBS-T, sections were counterstained with DAPI and mounted using CC/Mount aqueous mounting medium (cat #C9369, Sigma-Aldrich).

### Chemicals

COG-133 trifluoroacetate salt (APOE-RA) was purchased from Sigma-Aldrich (cat #C8624) and dissolved in PBS for i.p. injections (1 mg/kg body weight). The dosage and duration of treatment were determined based on previous studies using the same APOE mimetic peptide in other disease models (Li F-Q, et al. J Pharmacol Exp Ther. 2010; 334:106-15; Singh K et al. J Biol Chem. 2011; 286:3839-50). To determine the localization of COG-133 after i.p. injection, N-terminus biotinylated COG-133 was synthesized by GenScript. Mouse brains were collected 5 min after the injection of biotinylated COG-133 and fixed with 4% PFA. To detect biotinylated COG-133 bound in the brain, 50 μm cryosectioned brain slices were incubated with HRP-conjugated streptavidin (1:1000, cat #NEL750001EA, Akoya Biosciences) followed by TSA plus Cyanine-5 to stain for biotin. To also label LRP1 by immunohistochemistry (as described above), slices were incubated in anti-LRP1 primary antibody (1:200, cat #ab92544, Abcam) followed by RP-conjugated secondary antibody and TSA plus Fluorescein (1:500 in TSA diluent buffer).

### Blood-Brain Barrier Permeability Test

Sodium fluorescein (100 μl of 100 mg/ml; cat #F6377, Sigma-Aldrich) or Evans blue (200 μl of 20 mg/ml; cat #E2129, Sigma-Aldrich) was administered via i.p. injection to control and PAE mice. Tracers were allowed to circulate the body for 30 minutes, and brains were perfused as described above. Brains were sectioned with a microtome (Leica VT1000 S) at 100 μm and imaged with a BX63 microscope.

### Electrophysiology

Coronal brain slices (300 μm) at the level of the motor cortex were prepared from P30-40 mice using a vibratome (VT 1200S) as described previously (Mohammad S et al. Nat Neurosci. 2020; 23:533-43, Fogarty M J, Klenowski P M, Lee J D, Drieberg-Thompson J R, Bartlett S E, Ngo S T, et al. Cortical synaptic and dendritic spine abnormalities in a presymptomatic TDP-43 model of amyotrophic lateral sclerosis. Sci Rep. 2016; 6:37968). Briefly, brain slices were transferred to a submerged recovery chamber with oxygenated (95% O2 and 5% CO2) artificial cerebrospinal fluid (ACSF) solution containing 125 mM NaCl, 2.5 mM KCl, 2 mM CaCl2), 2 mM MgCl2, 25 mM NaHCO3, 1 mM NaH2PO4-H2O, and 25 mM glucose at room temperature for at least 1 h. Individual slices were then put into a recording chamber in which bath ACSF solution was continuously perfused at 32-33° C. Bath ACSF solution was obtained by omitting MgCl2 from the ACSF solution. Patch-clamp recordings were made from single neurons visualized with infrared differential interference contrast optics (BX51WI, Olympus, Tokyo, Japan). Whole-cell patch-clamp recordings were made from layer V pyramidal neurons in the motor cortex. Pyramidal neurons were identified based on their large size and teardrop morphology. The patch pipettes (2-5 MΩ) were filled with a cesium-based intracellular solution containing 130 mM Cs+-gluconate, 10 mM CsCl, 1 mM CaCl2, 11 mM EGTA, 2 mM ATP-Mg, and 10 mM HEPES (pH 7.2) for recordings of excitatory postsynaptic currents (EPSCs). sNMDA EPSCs were recorded under voltage-clamp conditions at a holding potential of +50 mV in the presence of 10 μM bicuculline, 5 μM strychnine, and 10 μM 1,2,3,4-Tetrahydro-6-nitro-2,3-dioxo-benzo[f]quinoxaline-7-sulfonamide (NBQX). The recording signals were amplified with a patch-clamp amplifier (MultiClamp 700B; Molecular Devices, CA, USA), low-pass filtered at 5 kHz, digitized with an analog-to-digital converter (Digidata 1440 A; Molecular Devices), and stored on a personal computer using a data acquisition program (Clampex 10.7; Molecular Devices) with a sampling frequency of 10 kHz. Synaptic events were analyzed offline with Clampfit 10.7 software (pCLAMP10, Molecular Devices). Peak, 10-90% rise times, and decay time constants (Tau) of EPSCs were calculated using standard algorithms. Only recordings with the access resistance<20 MΩ and less than 20% change throughout the experiment were included for analysis.

### Immunoblotting of CREB

Motor cortex, cerebellum, and striatum tissues were dissected from mice and frozen immediately in liquid nitrogen and stored at −80° C. until use. Brain tissues were homogenated in ice-cold T-PER reagent (cat #78510, Thermo Fisher Scientific) containing Halt protease and phosphatase inhibitor cocktail (cat #78430, Thermo Fisher Scientific) and EDTA for 15 s on ice. Tissue lysates were subsequently centrifuged at 10,000×g for 5 min at 4° C. The soluble portion of the lysates was collected for analysis.

Each protein sample was separated on NuPAGE 4-12% Bis-Tris gel (cat #NP032, Thermo Fisher Scientific). The separated proteins were transferred onto a PVDF membrane, probed with respective primary antibodies, and exposed to an HRP-conjugated secondary antibody. The following primary antibodies were used: anti-CREB (48H2) rabbit monoclonal antibody (cat #9197, Cell Signaling Technology, MA, CA), and anti-phospho-CREB (Ser-133) rabbit monoclonal antibody (cat #9198, Cell Signaling Technology). The reactive protein bands were visualized by chemiluminescence with the Amersham Imager 680 (GE Healthcare, IL, US) using the Super Signal West Pico Plus Chemiluminescent Substrate (Thermo Fischer Scientific). Subsequently, membranes were stripped with Restore Western Blot Stripping Buffer (cat #21059, Thermo Fisher Scientific) and reprobed with the mouse monoclonal antibody against GAPDH (cat #sc-47724, Santa Cruz, CA, US), followed by the same procedure to detect the reactive protein bands. Quantification of bands was done by densitometry using FIJI/Image J. For the analysis of CREB phosphorylation, the optical densities of phosphorylated CREB bands were measured relative to those of total CREB from the same brain sample. Statistical analysis was done with Student's t-test, and the threshold of significance was defined as P<0.05.

### RNAscope In Situ Hybridization

To detect mRNA molecules, RNAscope in situ hybridization was performed on PFA-fixed 20 μm-thick sections following the manufacturer's protocol. Apoe probes (cat #313271, Advanced Cell Diagnostics, CA, USA) were detected with TSA plus Fluorescein, followed by immunohistochemistry for NeuN by incubating the sections with an anti-NeuN primary antibody (1:200, cat #MAB377, EMD Millipore, MA, US), HRP-conjugated secondary antibody, and TSA plus Cyanine-5. Grin2a (cat #481831, Advanced Cell Diagnostics) and Grin2b (cat #417391-C2, Advanced Cell Diagnostics) probes were detected with TSA plus Fluorescein and TSA plus Cyanine-5, respectively. All samples were counterstained with DAPI before placing coverslips.

### Image Acquisition and Analysis

All images were taken by confocal microscopy (FV1000, Olympus). Allen Brain Atlas (mouse.brain-map.org/static/atlas) was used to define different brain regions visualized by DAPI staining. APOE, GFAP, GluN1, and LRP1-positive cells were quantified by first manually identifying DAPI-positive cells that are also immunolabeled, and then counting those immunolabeled cells within a region of interest using the cell counter plugin in ImageJ. To quantify the levels (total intensity) of APOE immunolabeling and biotinylated COG-133 detection per cell, the areas of individual cells were isolated and the staining intensity within each area was measured using Image J.

To count KCNN2 and PSD-95 puncta, automatic particle counting was performed using the particle analysis plugin in ImageJ. A colocalization tool in cellSens was used for colocalization analysis, and colocalization coefficients were reported.

Images of RNAscope in situ hybridization were analyzed following the method provided by the manufacturer. First, the total numbers of positive mRNA molecular signals and DAPI-positive cells were quantified separately using the automatic particle counting tool in ImageJ. Then the total number of mRNA puncta was divided by the total number of DAPI-positive cells to obtain the average number of mRNA molecules per cell.

Generation of Simulated Datasets and Comparison of Effect Sizes with the Real Data of APOE Staining

To investigate the underlying causes of decreased APOE levels in (NeuN+) cortical neurons and (GFAP+) astrocytes in PAE mice—whether they are due to an overall decrease in APOE levels in all neurons/astrocytes (Uniform Reduction model) or loss of neurons/astrocytes with high levels of APOE (Selective Cell Loss model)—the simulated dataset of APOE expression levels corresponding to each model was juxtaposed against the actual data of PAE mice. The datasets corresponding to the Uniform Reduction model were created by multiplying the actual data of APOE levels in control mice by a fixed ratio so that the mean APOE level values of simulated data matched those of the actual data of PAE mice. The dataset corresponding to the Selective Cell Loss model was formulated by excluding the data points of top 16 neurons and the top 11 astrocytes with the highest APOE levels from the actual data of control mice, so that the mean APOE level values of these simulated datasets matched those of the actual data of PAE mice.

The formula used for calculation of Cohen's d effect size is d=M2−M1/SDpooled. M1 is the mean of the actual data of PAE mice or simulated data, and M2 is the mean of the actual data of control mice. SDpooled is the pooled standard deviation.

### ATAC-seq

Cortical pyramidal neurons in the motor cortex were labeled with GFP that was introduced by in utero electroporation as described previously (Mohammad S et al. Nat Neurosci. 2020; 23:533-43). The pCAGIG plasmid was transferred into the motor cortex of E15 mouse embryos. Following electroporation, the pregnant mother received i.p. injection of ethanol at 4.0 g/kg weight or PBS daily at E16 and E17. At P15, the motor cortex was dissected and dissociated into single cells with Papain Dissociation System (cat #LK003150, Worthington, OH, US). The cells were FACsorted to collect GFP-positive cells. Dead cells were removed with SYTOX Red Dead Cell Stain (cat #S34859, Thermo Fisher Scientific). 500 cells in each group were tagmented with Illumina Tagment DNA Enzyme and Buffer Small Kit (cat #20034197, Illumina).

For 500-cell-low-input ATAC-seq, a previously published protocol was utilized (Corces M R et al. Nat Methods. 2017; 14:959-62). Lysis and transposition were conducted simultaneously with 10 μl of transposition mix (3.3 μl PBS, 1.15 μl water, 5 μl 2×TD Buffer, 0.25 μl Tn5 enzyme, 0.1 μl 1% digitonin, 0.1 μl 10% Tween-20, and 0.1 μl 10% NP40) at 37° C. for 30 min in a thermomixer shaking at 1000 RPM. Tagmented DNA was cleanup with a DNA Clean and Concentrator-5 kit (cat #D4004, Zymo Research, CA, USA) and amplified by PCR with index primers for 20 cycles. Purified libraries with Ampure XP (cat #A63880, Beckman Coulter, CA, USA) were sequenced with NovaSeq 6000 System (Illumina) as 2×50 bp paired-end readings.

The sequencing reads were aligned to the mouse reference genome (GRCm38/mm10) using Bowtie2 v2.3.4.3 (Langmead B et al. Nat Methods. 2012; 9:357-9) with parameters—very-sensitive—k 10). Mapped reads were filtered with Picard v2.25.0-0 (broadinstitute.github.io/picard/) for PCR duplicates, Samtools v1.12 (Li H et al. Bioinformatics. 2009; 25:2078-9) for mitochondrial DNA, and Bedtools v.2.30.0 (Quinlan A R et al. Bioinformatics. 2010; 26:841-2) for ENCODE blacklist (Amemiya H M et al. Sci Rep. 2019; 9:9354). Multimapreads were removed using the grep command with the “XS” tag in SAM format. The distribution of aligned fragment length was analyzed with Qualimap v2.2.2 (Okonechnikov K et al. Bioinformatics. 2016; 32:292-4). ATAC-seq peak regions were called using Genrich v0.6 (github.com/jsh58/Genrich) with parameters -j -y -r -v, and coverage tracks were drawn with Deeptools v3.5.0 (Ramirez F et al. Nucleic Acids Res. 2014; 42:W187-91) and SparK v2.6.2 (Kurtenbach S et al. bioRxiv. 2019. Preprint doi.org/10.1101/845529), normalized by library size with count per million (CPM). Gene annotations were added with ChlPseeker v1.26.2 (Yu G et al. Bioinformatics. 2015; 31:2382-3) in R v4.0.3.

### Genome-Wide Association Study

Saliva was collected from participants across four sites (San Diego and Los Angeles, CA; Atlanta, GA; Minneapolis, MN) as part of the ongoing international consortium, the Collaborative Initiative on Fetal Alcohol Spectrum Disorders (CIFASD). All sites have approval from their individual Institutional Review Board. Informed consent was provided by all participants and/or their parents or legal guardians.

Genome-wide single nucleotide polymorphisms (SNPs) were genotyped by the Johns Hopkins SNP Center on the Illumina OmniExpress array (n=234) or the Mega Consortium array (n=313). Individual genotype and SNPs were cleaned according to standard procedures and then imputed using the Michigan Imputation Server (Das S et al. Nat Genet. 2016; 48:1284-7) and the 1000 Genomes Reference panel. All SNP data are from genome build GRCh37/hg19. Exclusion criteria for individual SNPs were imputed information score<0.4, monomorphic SNPs, and SNPs with genotyping rates<99% across all genotyped samples. Principal components (PCs) estimating a continuum of allele frequency variation were calculated using SNPRelate (Zheng X et al. Bioinformatics. 2012; 28:3326-8). Using 1000 Genome Project (1KGP) (Auton A et al. Nature. 2015; 526:68-74) as a reference population, we grouped the participants based on their genetic ancestry: African American, European American, and American Hispanics from Mexico, Puerto Rico, Columbia, and Peru. Participants from other genetic ancestry groups were too few to be analyzed and excluded from the analysis. SNPs included in analyses had a minor allele frequency (MAF)≥0.01 and Hardy-Weinberg Equilibrium p-value≥0.000001 within each genetic ancestry group.

Using an additive genetic model, PLINK 2 (Chang C C et al. Gigascience. 2015; 4:7) was used to perform genome-wide association study (GWAS) analyses on the three ancestry groups separately. Covariates included sex, age at the time of the neuropsychological evaluation, the first 4 PCs, prenatal alcohol exposure (PAE), and the interaction of genotype*PAE. P-values for the genotype*PAE interaction were combined in a meta-analysis across the three ancestry groups using METAL (Willer C J et al. Bioinformatics. 2010; 26:2190-1). To associate the phenotypes, GWAS was performed using the delayed matching to sample task (DMS) total z-score from the Cambridge Neuropsychological Test Automated Battery (CANTAB) using PLINK 2 with the covariates defined above. Significant (p<0.05) SNPs associated with any of these three phenotypes across all three ancestry groups were examined. LocusZoom was used to visualize GWAS results near the APOE locus (Pruim R J et al. Bioinformatics. 2010; 26:2336-7).

### APOE Plasma Test in Ukraine Cohort

Plasma was collected from participants across two sites in Western Ukraine (Khmelnytsky Perinatal Center and Rivne Regional Medical Diagnostic Center) as part of the ongoing international consortium, the Collaborative Initiative on Fetal Alcohol Spectrum Disorders (CIFASD). Research and consent were approved by Institutional Review Boards at the University of California San Diego and Liviv Medical University in Ukraine. Informed consent was provided by all participants and/or their parents or legal guardians.

Pregnant women were recruited based on self-reported drinking behavior (Coles C D et al. Matern Child Health J. 2015; 19:2605-14). In the alcohol consuming group, women reported drinking either during the month of conception or the most recent month of pregnancy at least: weekly binge drinking (5+ drinks; five instances of 3-4 drinks; or 10 instances of 1-2 standard drinks). In the nonconsuming group, women reported no binge drinking, minimal to no drinking at conception, and no drinking in the most recent month of pregnancy.

Children were assessed at 6 and 12 months using the Bayley Scales of Infant Development (2nd edition) with standard scores for Mental Development Index (MDI), Psychomotor Development Index (PDI). Whole blood was collected in an EDTA-treated tubes Samples were centrifuged and plasma was aspirated, aliquoted, and stored in a −80° C. freezer. Samples were shipped on dry ice and store at −80° C. until analysis. Measurements of APOE (cat #3712-1HP-2, Mabtech) were carried out following the manufacturer's protocols.

Children whose mothers have a smoking history, obesity before pregnancy (BMI greater than or equal to 30), or with low birth weight (birth weight less than 2800×g) were excluded from analysis. Analysis was performed using SPSS software (SPSS version 28.0). Inverse probability weighting (IPW) was employed to reduce the selection bias. Four variables from maternal characteristics (age, socioeconomic index score, BMI before pregnancy, and recruitment site) and 4 variables from child characteristics (age, birth weight, sex, and developmental delay) were used. Propensity scores were calculated with the logistic regression to predict the probability of each mother consuming alcohol. The propensity score-based IPW balanced the baseline characteristics between alcohol exposed and non-exposed groups.

### Statistical Analysis

For all in vivo experiments, mice were excluded when health concerns were found, such as infection, bleeding, or significant body weight changes. All of the statistical analysis was carried out with GraphPad Prism 7.01. The D'Agostino-Pearson was performed to check for data normality. For the data that passed the normality test, two-tailed Student's t-tests or one-way or two-way ANOVAs were used and followed by a Tukey's or Bonferroni's post hoc test as described in the brief description of the drawings. Simple main effects were reported when there was a statistically significant interaction between independent variables by two-way ANOVA. The two-tailed Mann-Whitney U test was used for two-group comparisons of data that did not follow a normal distribution. Pearson's or Spearman's correlation coefficient calculation was done for normally or nonnormally distributed data, respectively. The local outlier factor (LOF) method was employed for outlier detection. There were no outliers in any of the data. P values of less than 0.05 were considered statistically significant. All of the statistical details of experiments can be found in the brief description of the drawings, including the statistical tests used and the exact value of n, which represents the number of individual animals. All data are expressed in mean s.e.m. The sample size for animal studies was determined based on previous experience with similar experiments and not on a statistical method. These animal studies were not performed in a blinded manner, and the investigators were aware of the group allocation during the experiment and when assessing the outcome.

### Results

Apoe Expression is Significantly Decreased in PBMCs of PAE Mice in Association with their Impaired Motor Learning

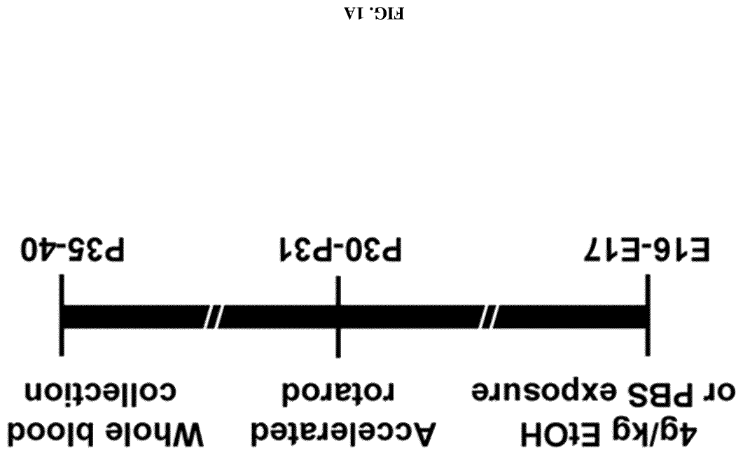

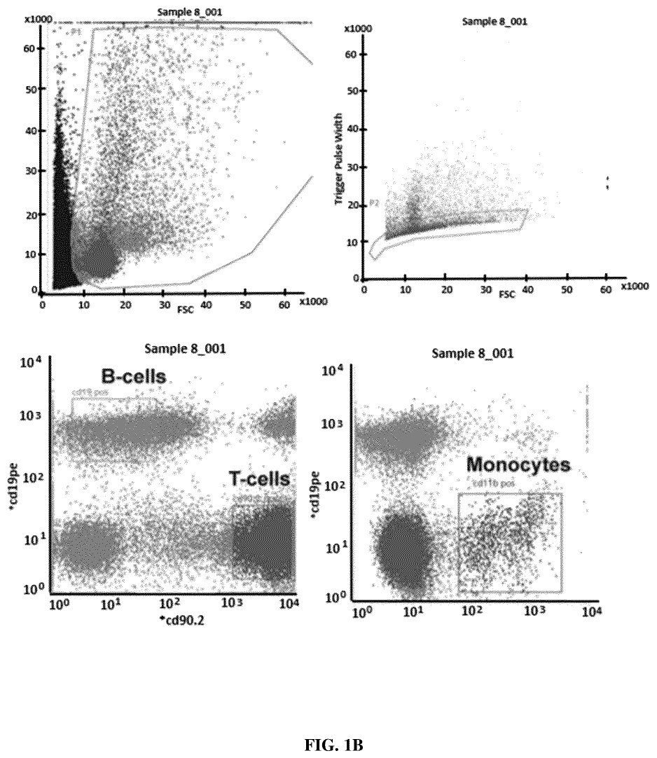

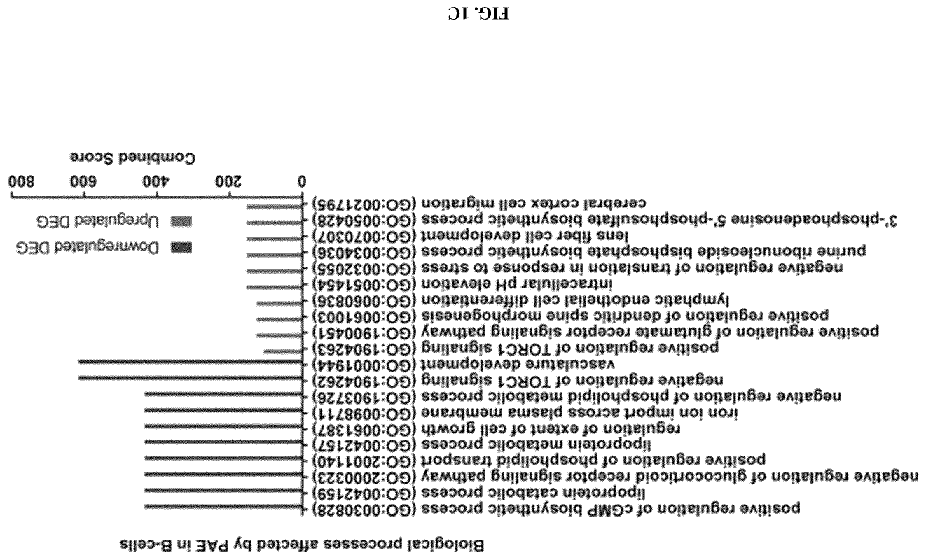

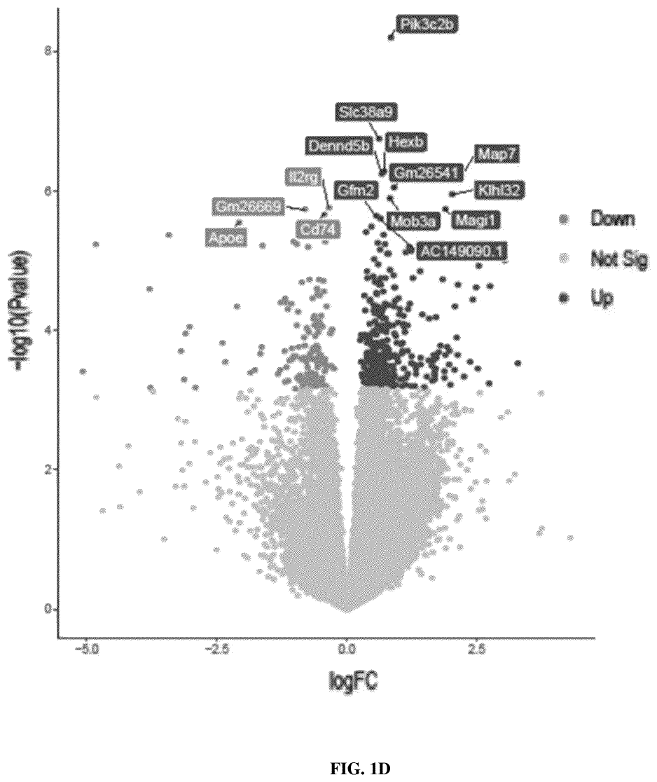

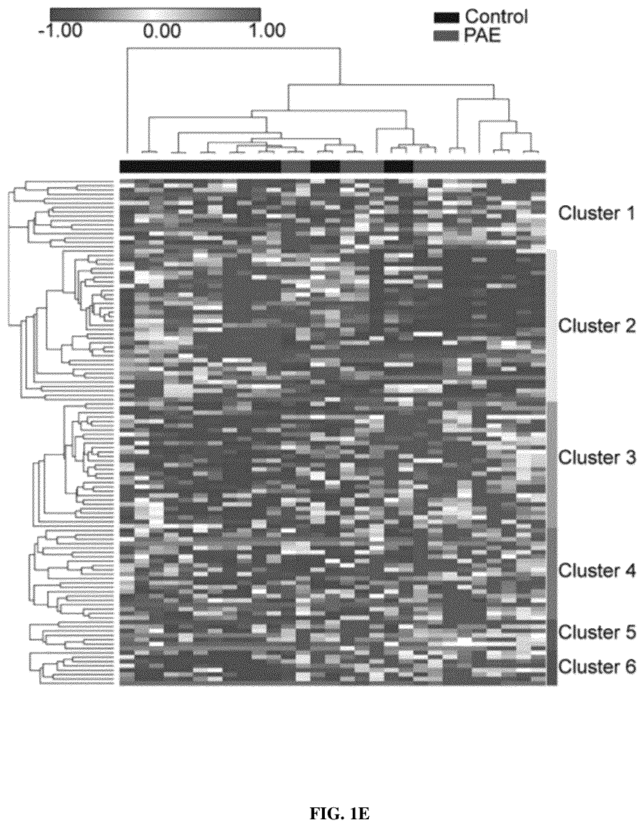

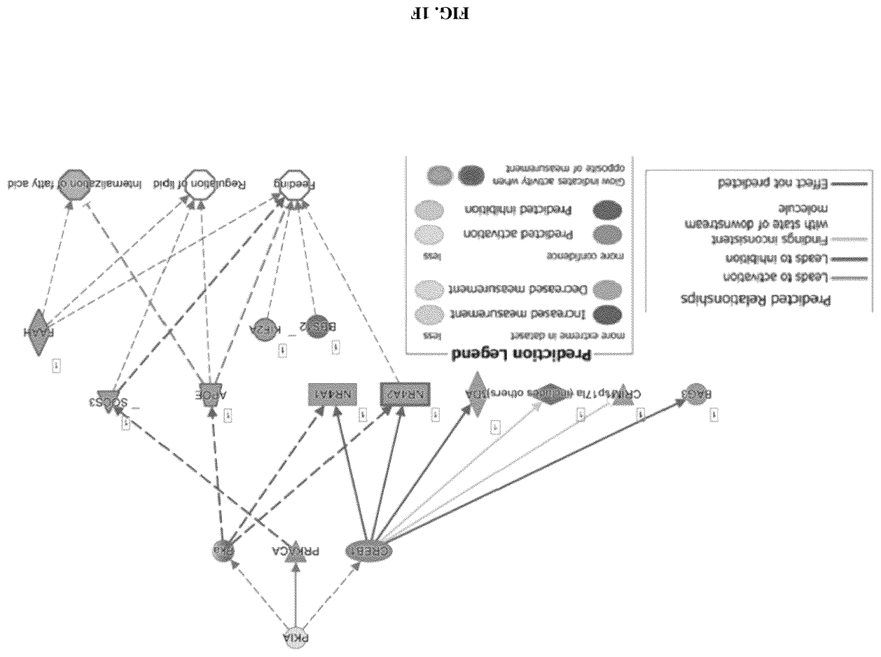

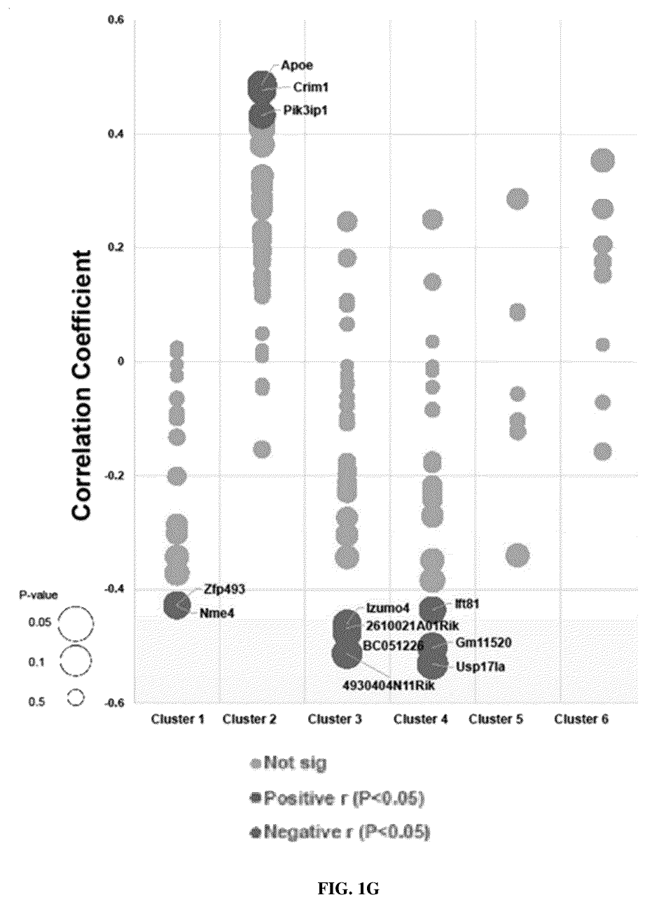

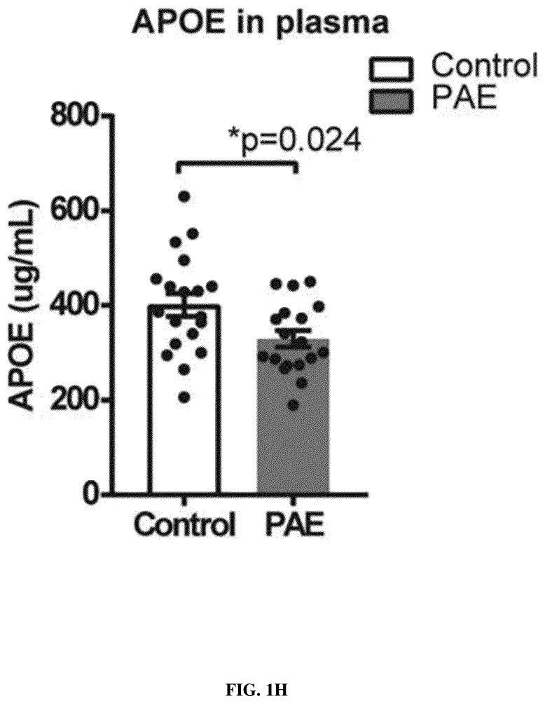

Growing evidence suggests that gene transcription measured in human blood correlates with transcripts measured in many other body systems, including the brain (Liew C-C, Ma J, Tang H-C, Zheng R, Dempsey A A. The peripheral blood transcriptome dynamically reflects system wide biology: a potential diagnostic tool. J Lab Clin Med. 2006; 147:126-32; Sullivan P F, Fan C, Perou C M. Evaluating the comparability of gene expression in blood and brain. Am J Med Genet B Neuropsychiatr Genet. 2006; 141B:261-8), indicating potential utilization of peripheral genomic information to decipher molecular changes in the brain which is difficult to get samples from. In fact, a previous study has demonstrated that genomic loci that are differentially methylated between PAE and control mice in the hypothalamus are highly similar to those in PBMCs (Lussier A A et al. Front Genet. 2018; 9:610). Therefore, RNA sequencing of PBMCs from PAE and control mice was performed to gain insights into the molecular changes in the brain attributable to PAE. Ethanol or PBS vehicle was administered to pregnant dams at embryonic day (E) 16 and 17 to generate PAE mice, as an animal model of FASD, and control mice, respectively (FIG. 1A, details in Materials and methods). This PAE regimen does not induce gross brain structural changes but elicits fine and gross motor learning deficits in mice (Mohammad S et al. Nat Neurosci. 2020; 23:533-43). Following the generation of PAE and control mice, an accelerated rotarod test was performed on postnatal day (P) 30 as previously done (Mohammad S et al. Nat Neurosci. 2020; 23:533-43) and whole blood was collected the on P31 to isolate PBMCs. Using fluorescence-activated cell sorting (FACSorting) with antibodies for CD19, CD90.2, and CD11b, B- and T-cells and monocytes were sorted, respectively (FIG. 1B).

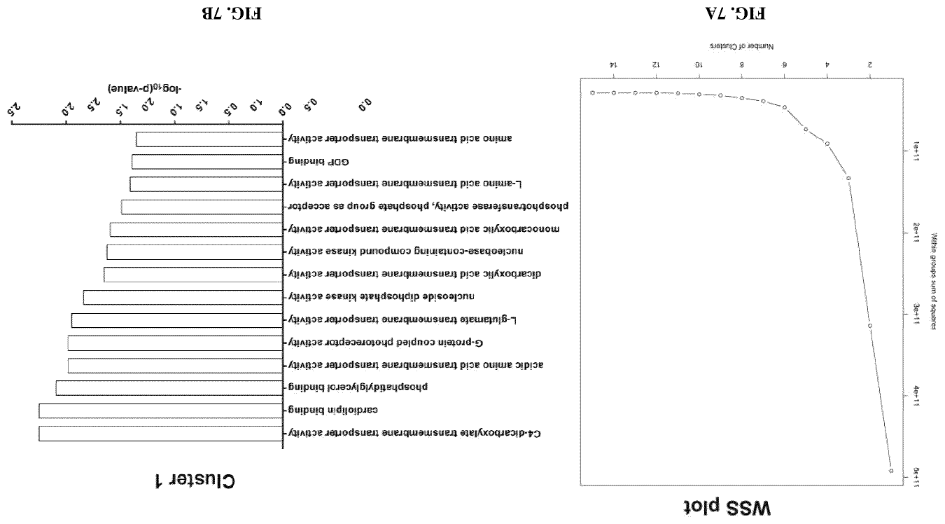

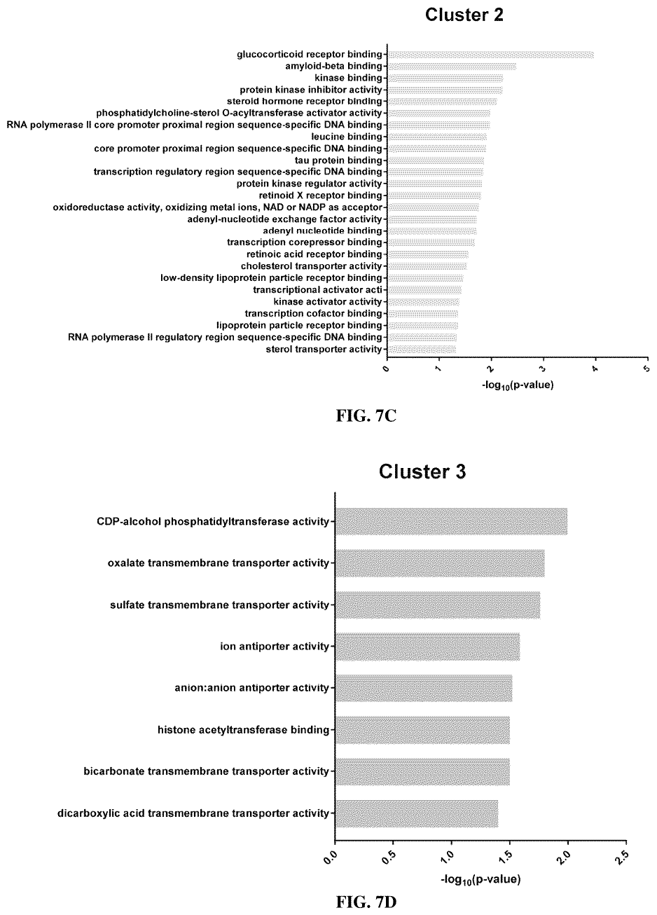

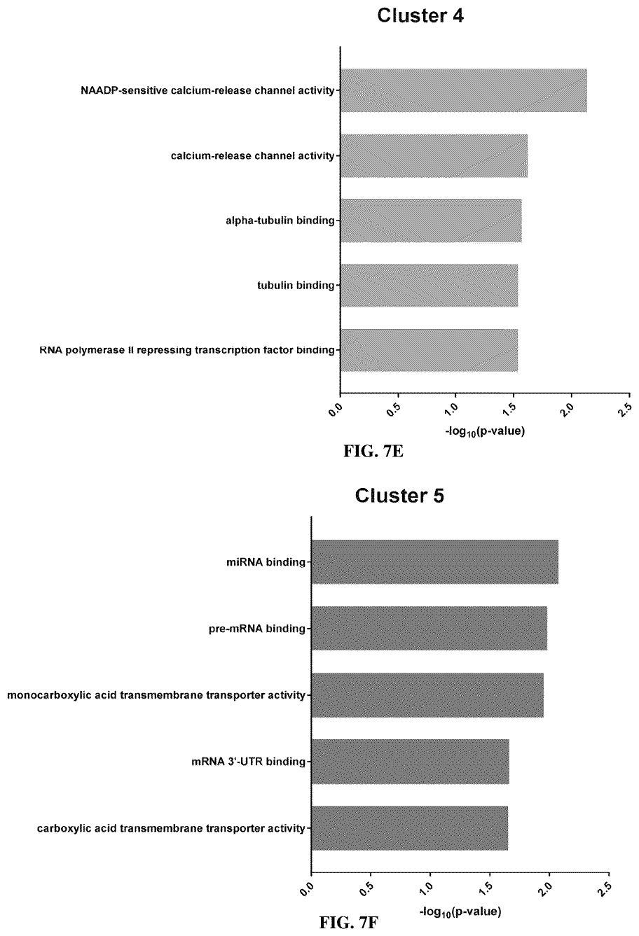

With limma-voom algorithms (Ritchie M E et al. Nucleic Acids Res. 2015; 43:e47-e47), differentially expressed genes (DEGs) between control and PAE mice were defined with a cut-off of twofold change and p value<0.05. No DEGs in monocytes were found, while 116 (35 downregulated and 81 upregulated in PAE) and 6,568 (178 upregulated and 6,390 downregulated in PAE) DEGs were found in B-cells and T-cells, respectively. Of note, enrichment analysis (enricher) showed that downregulated DEGs in B-cells were enriched in lipoprotein metabolism processes and phospholipid-related pathways (FIG. 1C). Among genes in these pathways, Apoe, which serves the major function of lipoprotein metabolism (Hauser P S et al. Prog Lipid Res. 2011; 50:62-74), was one of the top genes that were significantly downregulated in PAE mice (FIG. 1D). K-means clustering of DEGs in B-cells was also performed by using the optimal number of clusters determined by the Within Sum of Squares method (FIG. 7A). As a result, DEGs were classified into 6 distinctive gene clusters (FIG. 1E). Gene Ontology (GO) enriched in each cluster was determined (FIGS. 7B-7G). In cluster 2 that included Apoe, lipid-related pathways such as cholesterol transporter activity and lipoprotein particle receptor binding were enriched (FIG. 7C). Similarly, Ingenuity Pathways Analysis (IPA) of cluster 2 suggested changes in the regulation of lipid and internalization of fatty acids in PAE mice (FIG. 1F).

The previous study found that PAE caused motor learning deficits in mice (Mohammad S et al. Nat Neurosci. 2020; 23:533-43). Therefore, a correlation analysis between the expression of the DEGs and the motor learning index (see “Materials and methods”) in PAE mice was conducted. It was found that Apoe expression in cluster 2 demonstrated the highest (positive) correlation with the motor learning index (FIG. 1G). Crim1, which encodes a transmembrane protein containing an insulin-like growth factor-binding domain and plays a role in organ development (Kolle G et al. Mech Dev. 2000; 90:181-93), and Pik3ip1, which encodes a protein that negatively regulates phosphatidylinositol 3-kinase (PI3K) to control neuronal survival and metabolism (Sánchez-Alegria K et al. Int J Mol Sci. 2018; 19:3725), were also significantly correlated with motor learning. Both were also from cluster 2 (FIG. 1G).

In T-cells, enrichment analysis also revealed that downregulated DEGs were enriched in lipid response, fat differentiation, MAP kinase, and cytokine signaling pathways (FIG. 8A). No significant GOs were identified with the list of upregulated DEGs in T-cells. The top regulator effect network identified by IPA included changes in lipid synthesis (data not shown). As both B- and T-cells showed changes in lipid-related pathways in their downregulated gene sets by PAE, the genes commonly downregulated between B- and T-cells were examined, and it was found that Apoe was one of six such genes (FIG. 8B). Consistent with the result of RNA sequencing, it was found that the level of circulating APOE in the plasma was significantly decreased in PAE mice compared to control mice at P30 (FIG. 1H).

### APOE Expression in the Motor Cortex is Reduced in PAE Mice

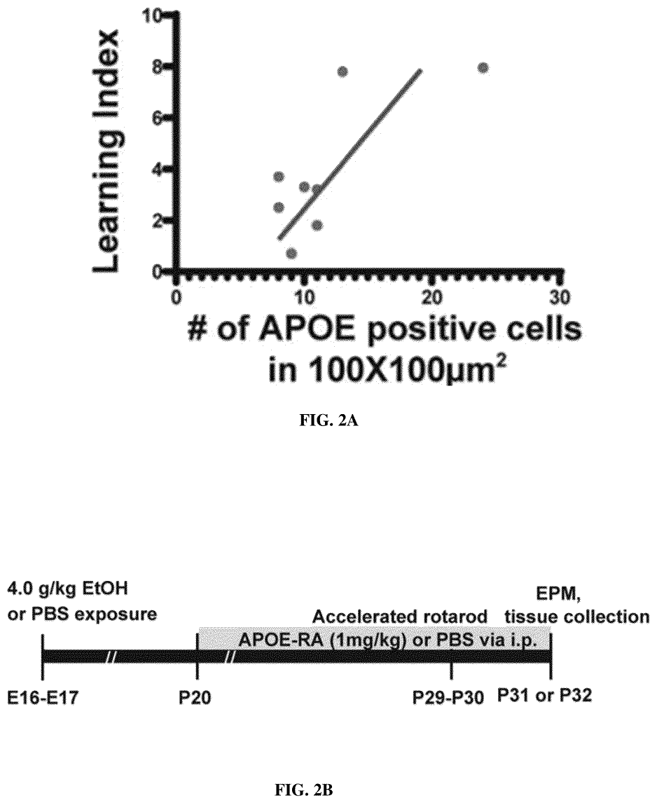

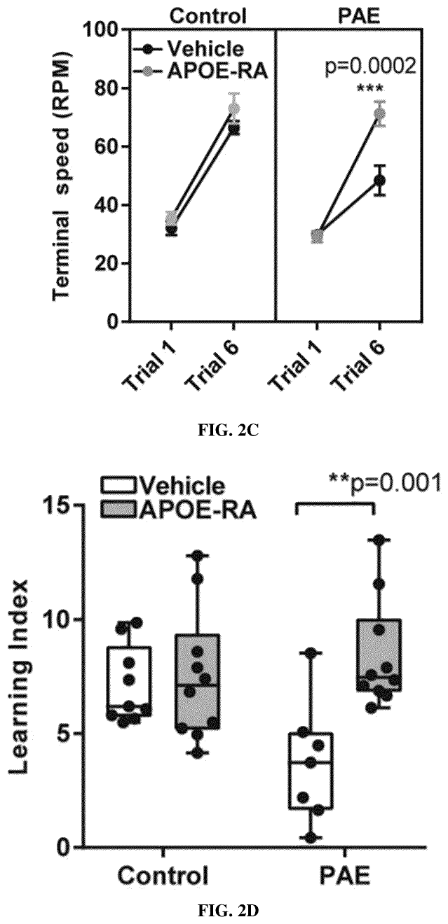

Based on the finding of an association between decreased APOE in peripheral blood and impaired motor learning in PAE mice, APOE expression in the motor cortex, one of the brain regions primarily responsible for motor learning, was next examined. Puncta-like expression of APOE by immunohistochemistry in normal mice consistent with previous reports (Konings S C et al. Life Sci Alliance. 2023; 6:e202201887; Liraz O et al. Mol Neurodegener. 2013; 8:16; Zalocusky K A et al. Nat Neurosci. 2021; 24:786-98; La Cunza N et al. JCI Insight. 2021; 6:e142254) was observed, and no immunolabeling was confirmed by the negative control procedure without including the primary antibody. Immunohistochemistry at P30 revealed a significant reduction of APOE-positive cells in the motor cortex in PAE mice compared to control mice (FIGS. 9A-9B). The adjacent cingulate cortex showed no difference in the number of APOE-positive cells between control and PAE mice (FIG. 9A). Correlation analysis further revealed that the number of APOE-positive cells in the motor cortex was positively correlated with the motor learning index in PAE mice, indicating that animals with fewer APOE-positive cells have poorer motor learning (R2=0.56, P=0.017) (FIG. 2A), consistent with the result of correlation analysis on B-cells (FIG. 1G).

Previous studies have shown that APOE is expressed in both neurons (Xu P-T et al. Am J Pathol. 1999; 154:601-11; Dekroon R M, Armati P J. Synthesis and processing of apolipoprotein E in human brain cultures. Glia. 2001; 33:298-305) and astrocytes (Boyles J K et al. J Clin Investig. 1985; 76:1501-13) in the brain. Double immunolabeling for APOE with neuronal nuclei (NeuN) or glial fibrillary acidic protein (GFAP) in both control and PAE mouse brain sections revealed that APOE was expressed in both NeuN-positive neurons and GFAP-positive astrocytes in the entire layer V/VI of the motor cortex (FIGS. 9C and 9E). No obvious changes in the brain structure and neuronal number in PAE mice were previously observed (Mohammad S et al. Nat Neurosci. 2020; 23:533-43). Similarly, no significant difference (P=0.34) in the number of GFAP-positive astrocytes between control and PAE mice was found (FIGS. 9G-9H), suggesting that the reduction in the number of APOE-positive cells in the brain of PAE mice is not due to changes in the number of astrocytes or neurons, but due to reduction in the APOE expression level in these cells.

Since a recent study using a similar PAE mouse model has demonstrated that PAE induces a small but significant increase in a specific type of apoptosis in the developing brain (László Z I et al. Nat Commun. 2020; 11:4363), it was further investigated whether the reduction in the number of APOE-positive cells in PAE mice is due to the loss of cells with high levels of APOE expression (Selective Cell Loss model) or APOE expression levels are decreased overall in all cells (Uniform Reduction model). It was first confirmed that both NeuN+ neurons and GFAP+ astrocytes show reduction of APOE immunolabeling in the motor cortex in PAE mice (FIGS. 9C-9F). To determine which model aligns with the case of PAE mice, two simulated datasets were created (FIGS. 9D and 9F). The simulated dataset corresponding to the Selective Cell Loss model was formulated by excluding data points of top 16 neurons and top 11 astrocytes with the highest APOE levels from the data of control (PBS-exposed) mice. These thresholds were set so that the mean APOE level values of these simulated datasets matched those of the data obtained from PAE mice. The datasets corresponding to the Uniform Reduction model were created by multiplying the APOE level data of control mice by a fixed ratio so that their mean APOE level values matched those of the data of PAE mice. Comparison of Cohen's d values relative to the data of control mice between the actual data of PAE mice and the simulated data of two models suggested that the APOE reduction in PAE mice is likely due to a uniform decrease in APOE expression in many cells rather than to the loss of high APOE-expressing cells, for both neurons and astrocytes (FIGS. 9D and 9F). However, these results do not completely exclude the possibility that the uniform reduction of APOE may encompass an adaptive response to PAE-induced cell death, given the role of APOE in neuronal death (Guttenplan K A et al. Nature. 2021; 599:102-7).

### APOE-RA Improves Motor Learning and Reduces Anxiety in PAE Mice

The PBMC RNA sequencing and immunohistochemical analysis of the PAE model supported the possibility that the hypoexpression of APOE by PAE in the brain could be involved in neurobehavioral changes in PAE offspring. In fact, Apoe knockout mice exhibit spatial (Lane-Donovan C et al. J Neurosci. 2016; 36:10141-50) and reversal learning deficits (Boyles J K et al. J Clin Investig. 1985; 76:1501-13) similar to those shown in another PAE mouse model and PAE patients (Marquardt K et al. Alcohol Clin Exp Res. 2014; 38:2962-8; Mattson S N, Crocker N, Nguyen T T. Fetal alcohol spectrum disorders: neuropsychological and behavioral features. Neuropsychol Rev. 2011; 21:81-101). One of the major APOE receptors, LRP1, is located at post-synaptic sites, and abolishing these receptors in neurons results in impaired motor function in mice (Liu Q et al. J Neurosci. 2010; 30:17068-78), suggesting that the APOE-LRP1 interaction is required for proper motor function.

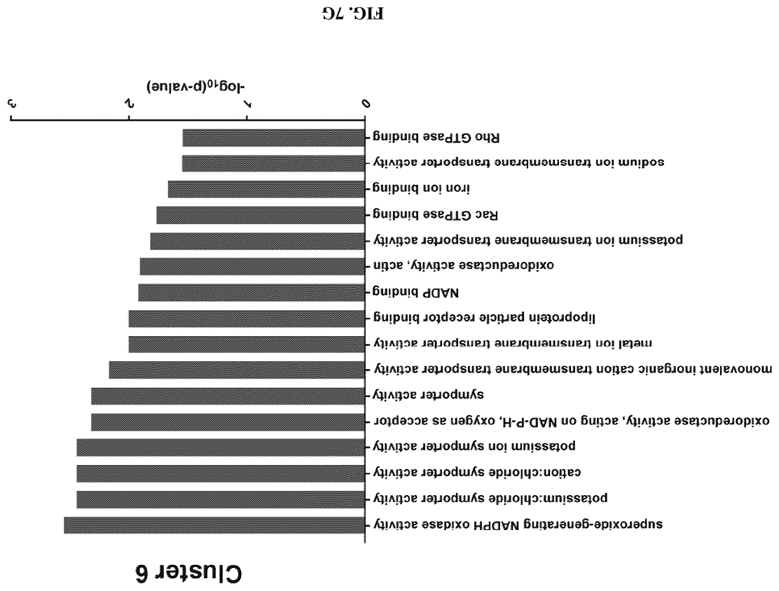

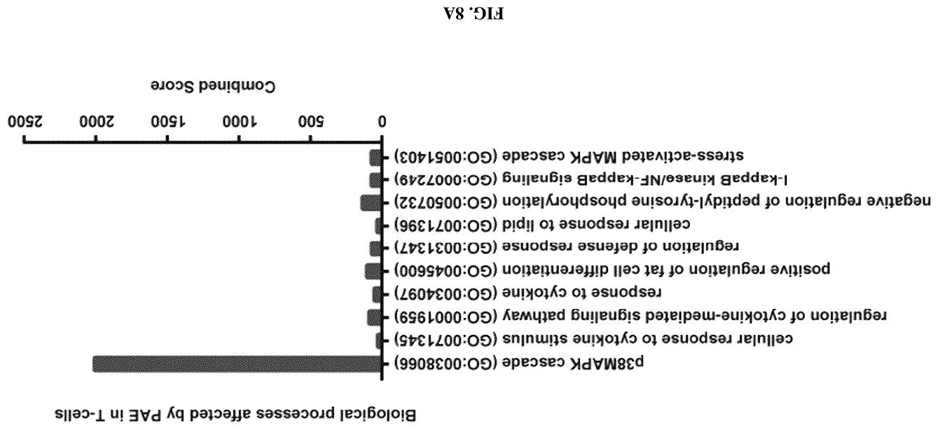

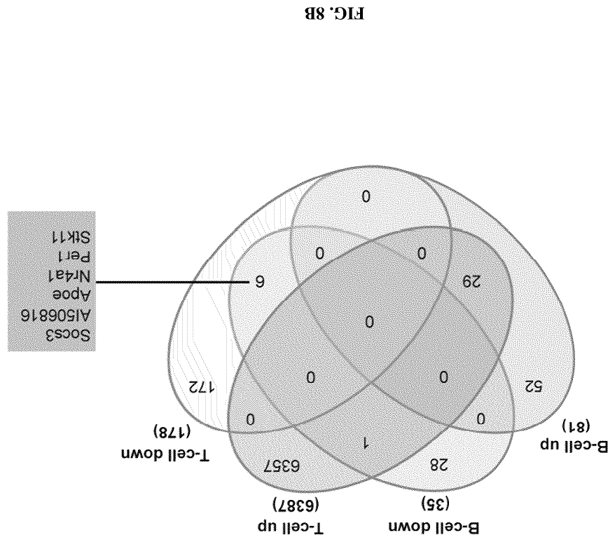

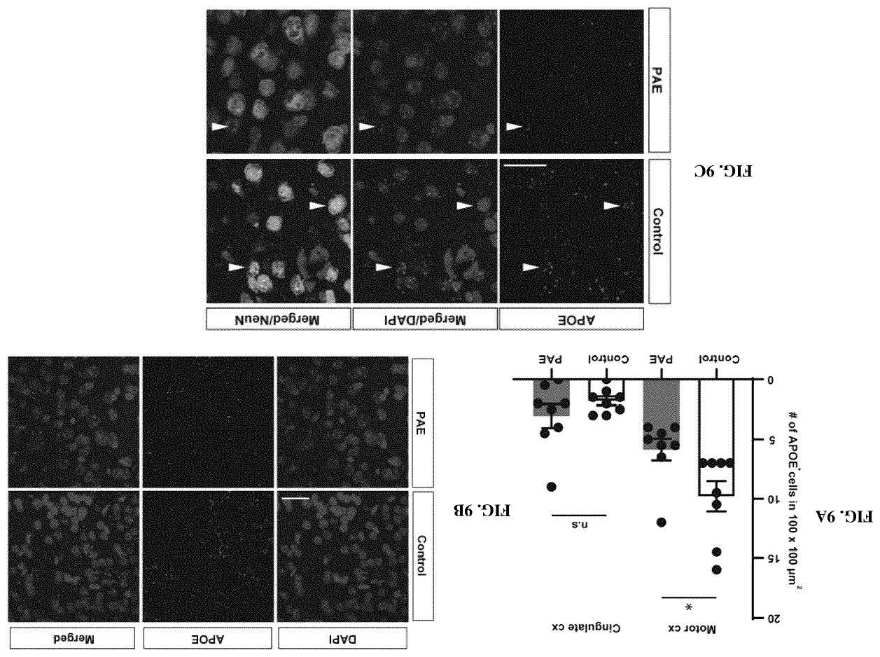

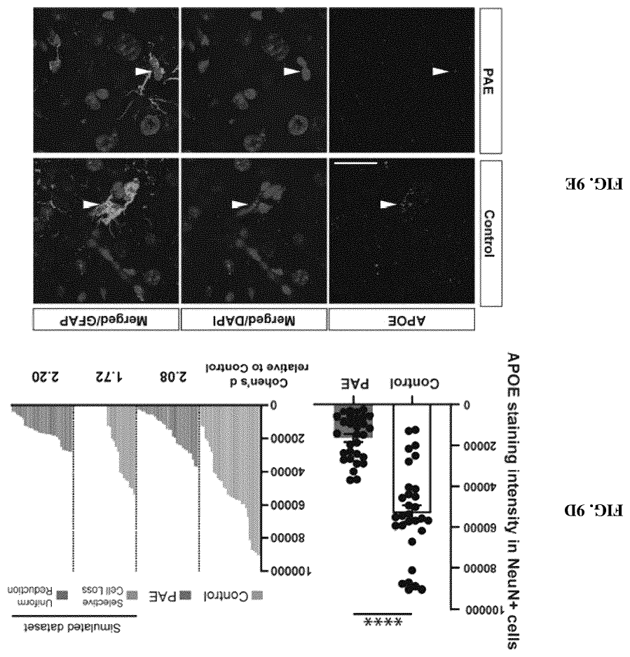

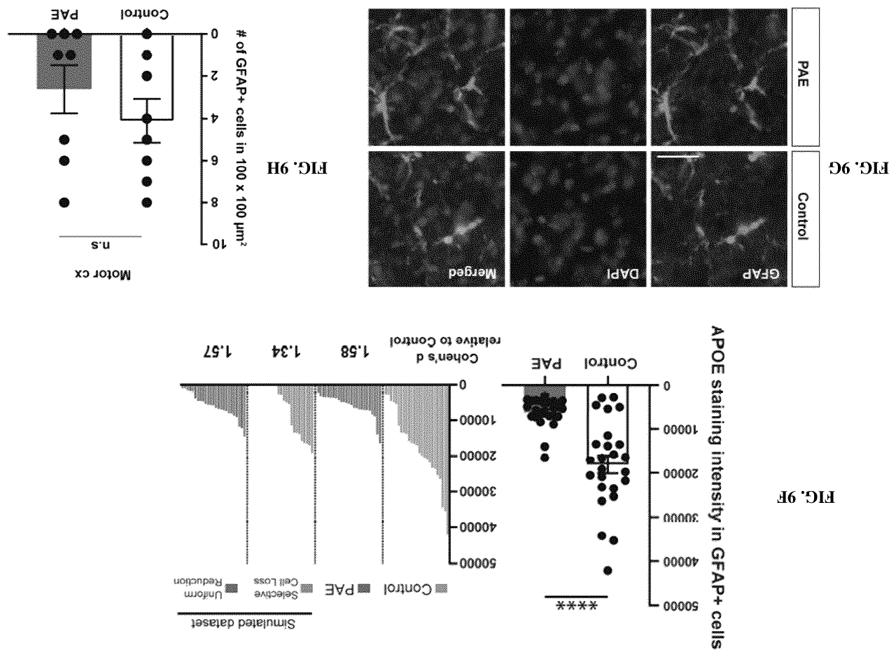

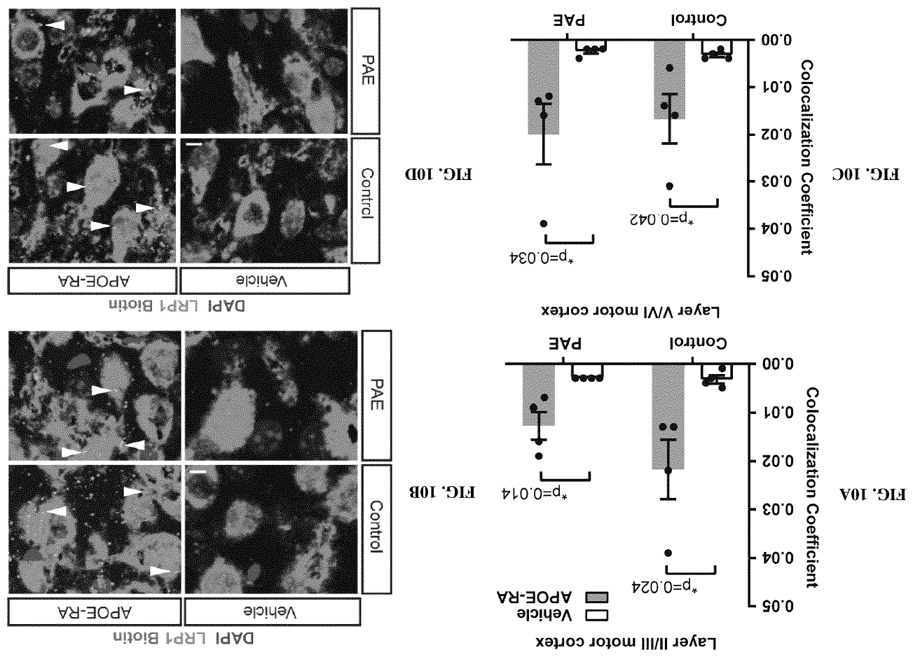

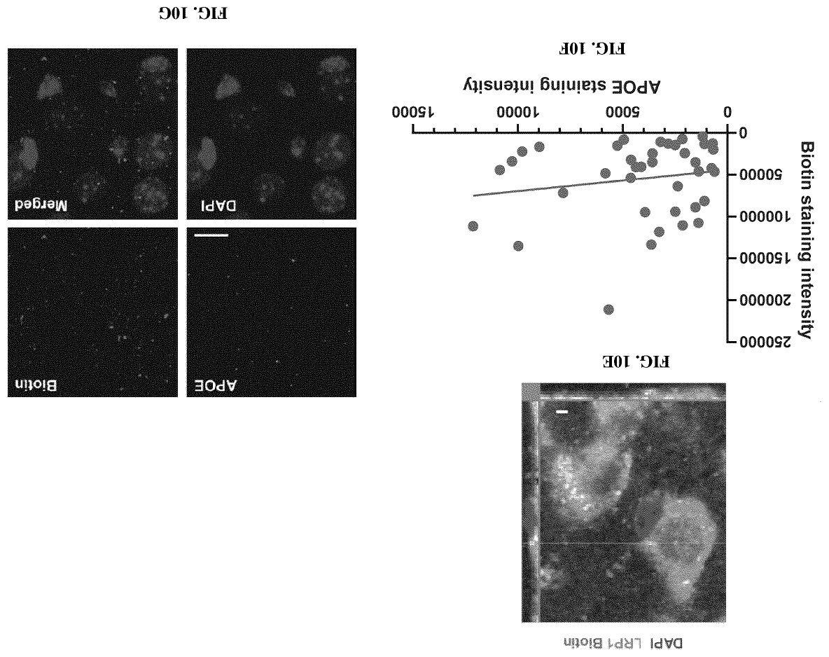

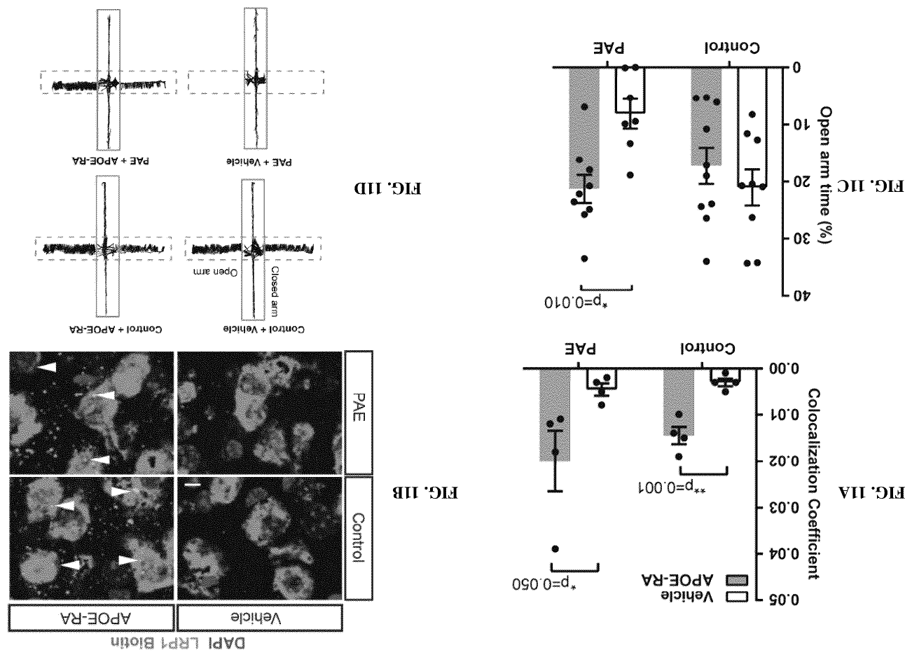

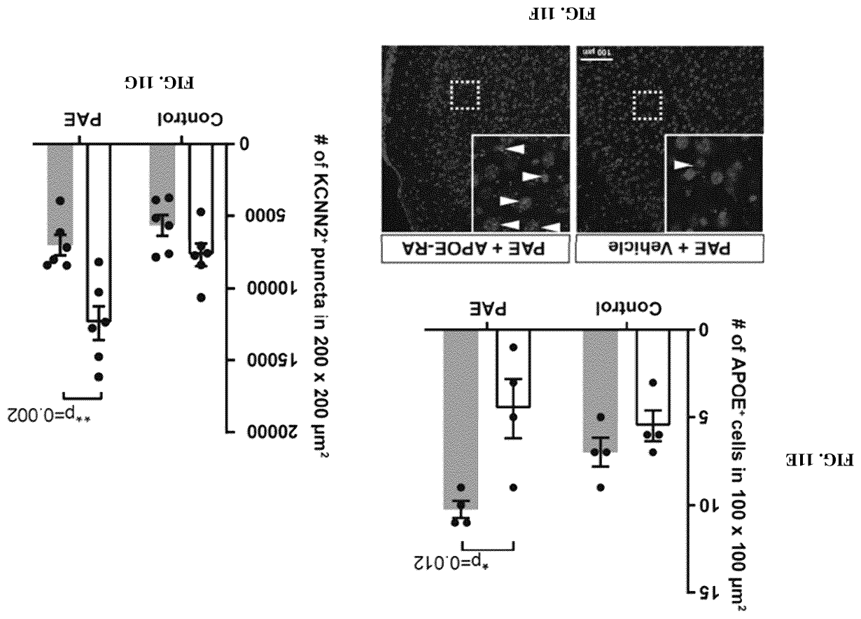

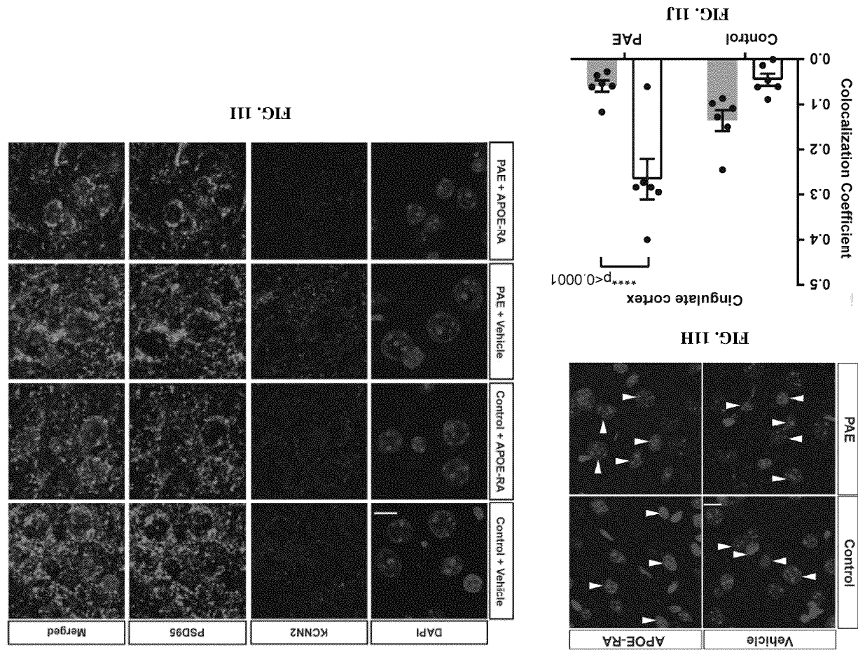

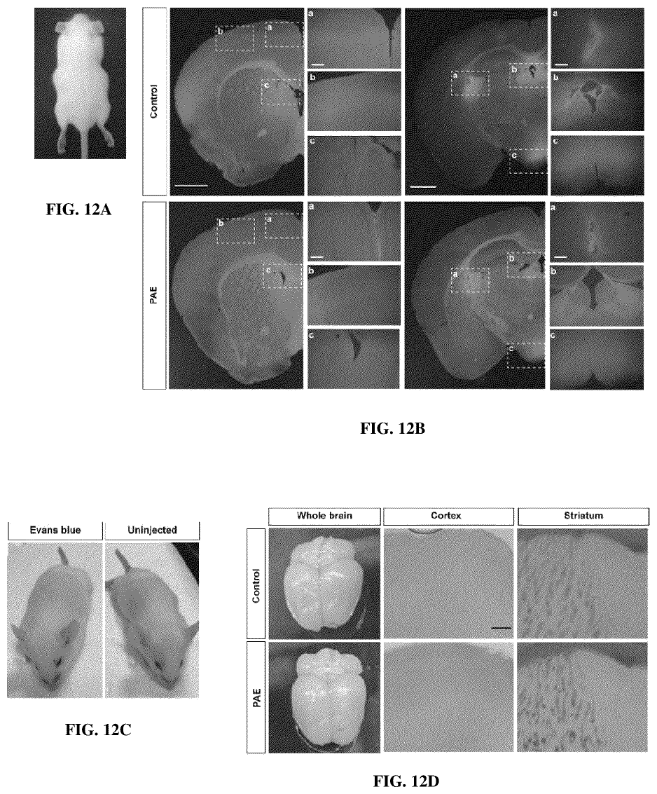

To examine whether application of APOE mitigates neurobehavioral deficits in PAE mice, APOE-RA (or COG 133) was tested, which contains the receptor binding unit of human APOE and binds to LRP1, but does not include the lipid-binding domain (Croy J E et al. Biochemistry. 2004; 43:7328-35). While full-length APOE does not cross the blood-brain barrier (BBB) (Liu M et al. Am J Physiol Regul Integr Comp Physiol. 2012; 303:R903-8), APOE-RA crosses it (Sarantseva S et al. PLoS ONE. 2009; 4:e8191). Therefore, it was first checked whether administered APOE-RA reached the target neurons in the motor cortex where a significant reduction of APOE-positive cells in PAE mice was observed (FIGS. 9A-9B). Biotinylated APOE-RA was injected intraperitoneally (i.p.) at 1.0 mg/kg body weight at P20, and brains were collected 5 minutes after the administration. Modified streptavidin-biotin staining detected the binding of APOE-RA to cells in the entire motor cortex, including LRP1-expressing cells in both layer II/III and layer V/VI (FIGS. 10A-10G). No significant correlation was found between the levels of biotinylated APOE-RA detection and (endogenous) APOE staining in individual cells in layer V/VI in PAE mice, suggesting that the effects of APOE-RA binding was not different among cells with different levels of endogenous APOE expression (FIGS. 10F and 10G). The biotinylated APOE-RA was also detected in other brain areas, including the cingulate cortex (FIGS. 11A and 11B). It was confirmed that BBB integrity was not compromised in PAE mice using two different dyes, sodium fluorescein, and Evans blue, at P30 (FIGS. 12A-12D).

To test the effect of APOE-RA on learning deficits in PAE mice, it was administered i.p. daily at 1.0 mg/kg body weight from P20 to P31 or 32 as depicted in FIG. 2B. First, the accelerated rotarod test was performed around P30. It was found that motor learning deficits in PAE animals were significantly improved by APOE-RA administration; the terminal speed at the last trial (trial 6) and the learning index were significantly higher in APOE-RA-treated PAE mice compared to vehicle-treated PAE mice (FIGS. 2C-2D). On the other hand, no significant differences were detected in motor learning behavior in control mice between APOE-RA and vehicle treatment, or in the body weight during the treatment between control and PAE mice (FIGS. 12E-12F).

24 h after the accelerated rotarod test, the same animals were placed on an elevated plus maze to assess anxiety. A previous study demonstrated that PAE mice spent a smaller percentage of total time in open arms than control mice (Hwang H M et al. Transl Psychiatry. 2022; 12:24). Consistently, the percentage of time spent in open arms by PAE mice was shorter than that by control mice without treatment, but the percentage was brought back to the control level by APOE-RA treatment (FIGS. 11C and 11D), suggesting that the anxiety behavior in PAE mice was also mitigated by APOE-RA treatment. In control mice, there was no APOE-RA effect on the open arm time (FIGS. 11C and 11D). Overall, these data demonstrate that decrease of APOE contributes to the motor learning deficit and anxiety behavior in PAE mice, and administration of APOE-RA positively mitigates both behavioral abnormalities.

### Decrease of APOE by PAE Alters NMDAR Function and Upregulates KCNN2 Expression

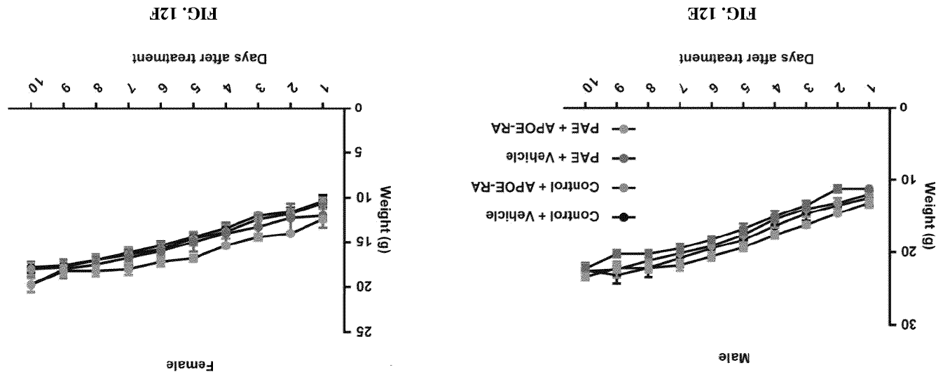

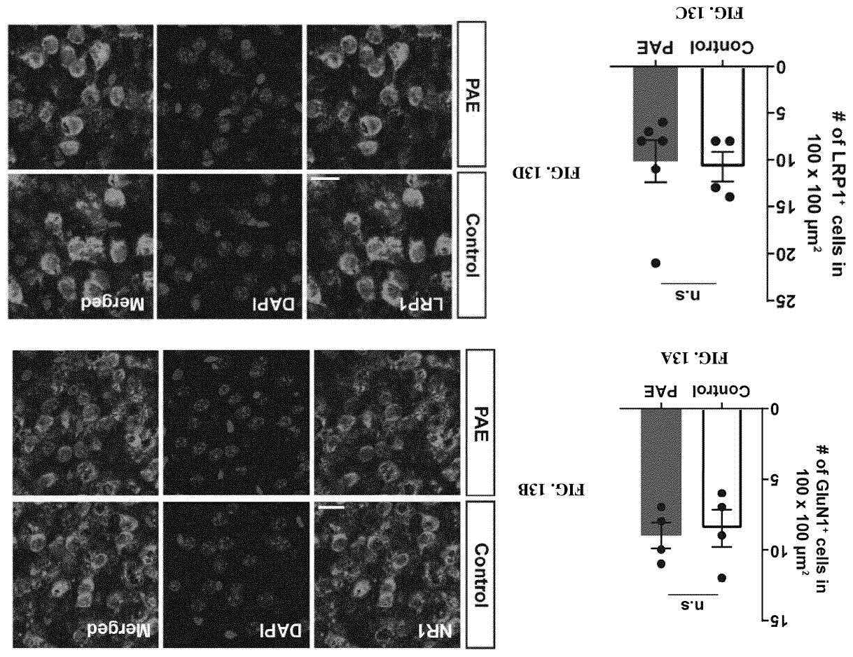

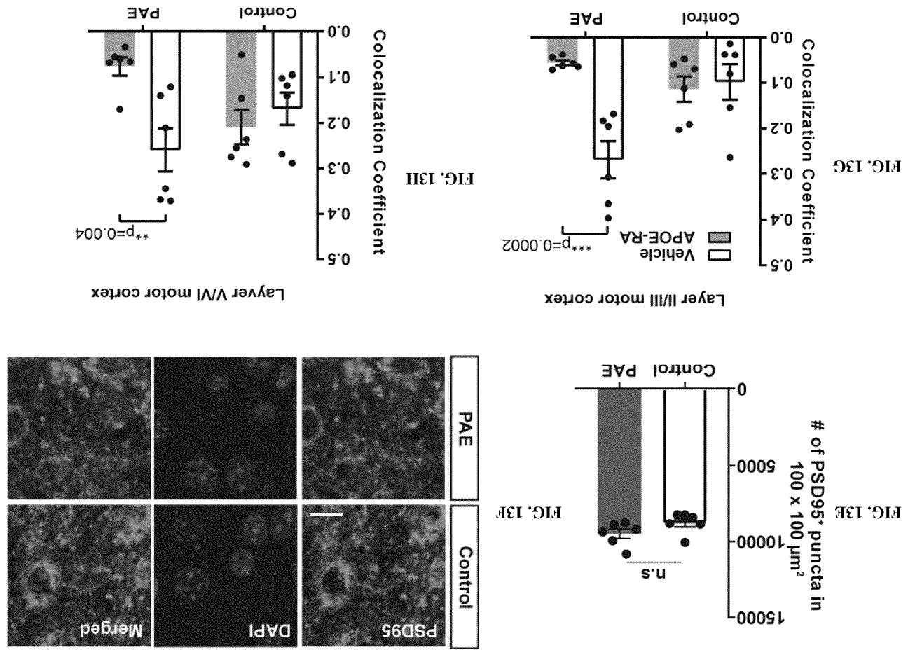

Next, the molecular pathways affected by reduction of APOE underlying neurobehavioral abnormalities in PAE mice was examined. In the motor cortex at P30, the effects of PAE on the expression levels and patterns of LRP1, as well as the NMDA receptor (NMDAR) and PSD-95 was examined, for which interactions with LRP1 at the postsynapse have been demonstrated (May P et al. Mol Cell Biol. 2004; 24:8872-83; Nakajima C et al. J Biol Chem. 2013; 288:21909-23). GluN1 antibody was used to label NMDARs as GluN1 is an obligatory subunit for the receptor (Paoletti P et al. Nat Rev Neurosci. 2013; 14:383-400) and its expression in the motor cortex was shown to be positively associated with motor learning in mice (Hasan M T et al. Nat Commun. 2013; 4:2258). However, no significant changes in any of those proteins were found (FIGS. 13A-13F), suggesting that PAE affects the neurobehavioral deficits by mechanisms other than the expression of these APOE-related synaptic proteins.

The previous study found that the expression of potassium intermediate/small calcium-activated channel, subfamily N, member 2 (KCNN2) was upregulated in the motor cortex of PAE mice, and knockdown of Kcnn2 expression in the motor cortex or pharmacological inhibition of KCNN2 function improved the motor learning deficits in PAE mice (Mohammad S et al. Nat Neurosci. 2020; 23:533-43). In the mouse model of Angelman syndrome that shows motor learning deficit and anxiety similar to the mouse model of FASD herein (Silva-Santos S et al. J Clin Investig. 2015; 125:2069-76; Miura K et al. Neurobiol Dis. 2002; 9:149-59; Huang H-S et al. Behav Brain Res. 2013; 243:79-90), malfunction of the feedback regulation between NMDA and KCNN2 pathways was shown to lead to excessive KCNN2 at synapses and cause intellectual disabilities (Sun J et al. Sci Rep. 2020; 10:9824). Other studies have also shown a functional regulation of NMDARs by LRP1 receptor activation through their coupling via PSD-95 (May P et al. Mol Cell Biol. 2004; 24:8872-83; Martin A M et al. J Biol Chem. 2008; 283:12004-13). Therefore, it was hypothesized that changes in LRP1-mediated NMDAR function due to reduced APOE underlie the increased expression and/or synaptic distribution of KCNN2 in PAE mice.

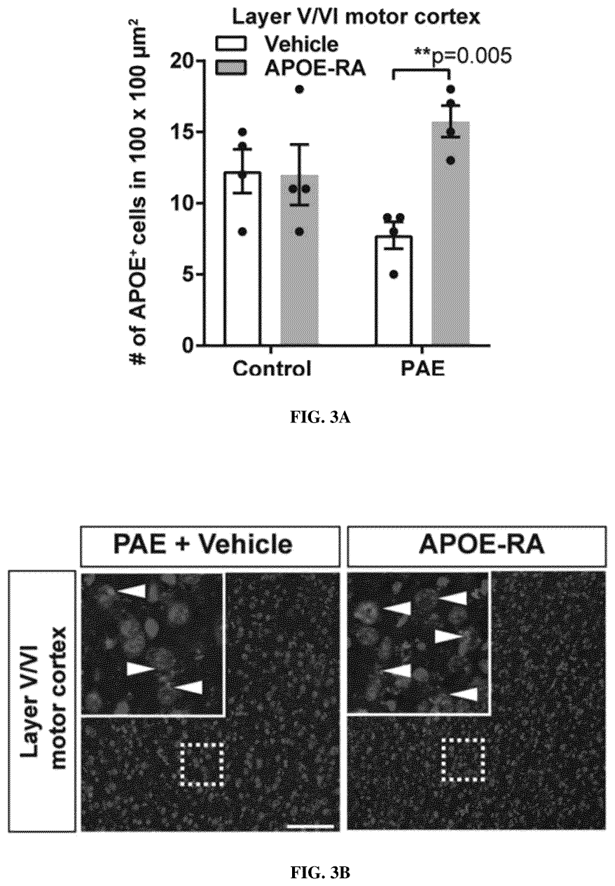

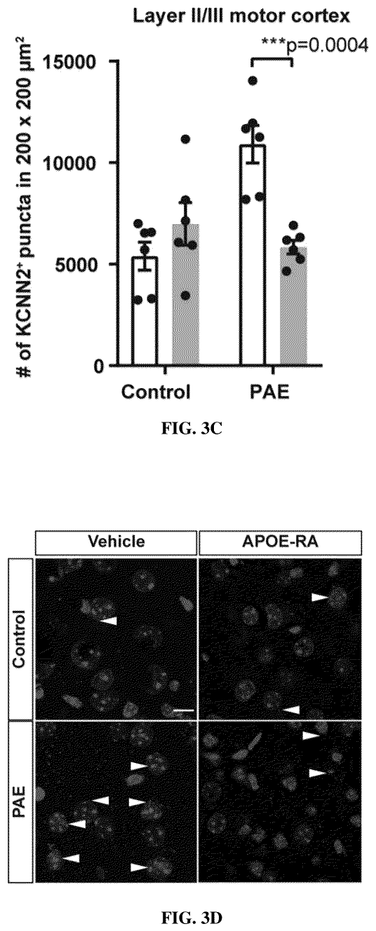

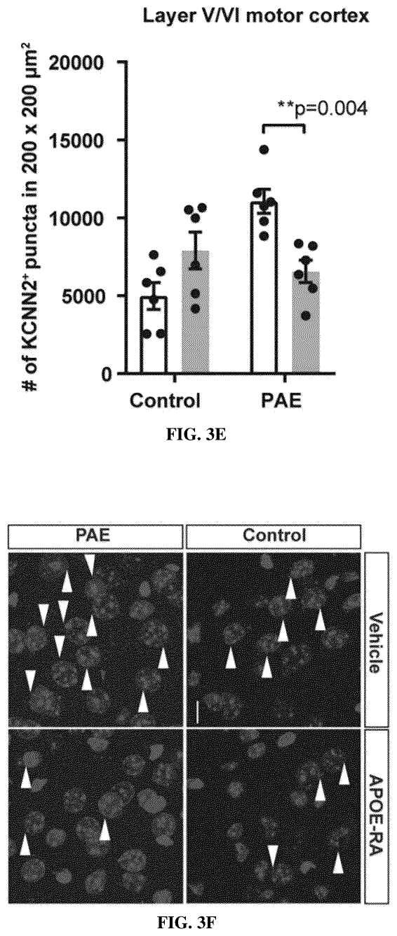

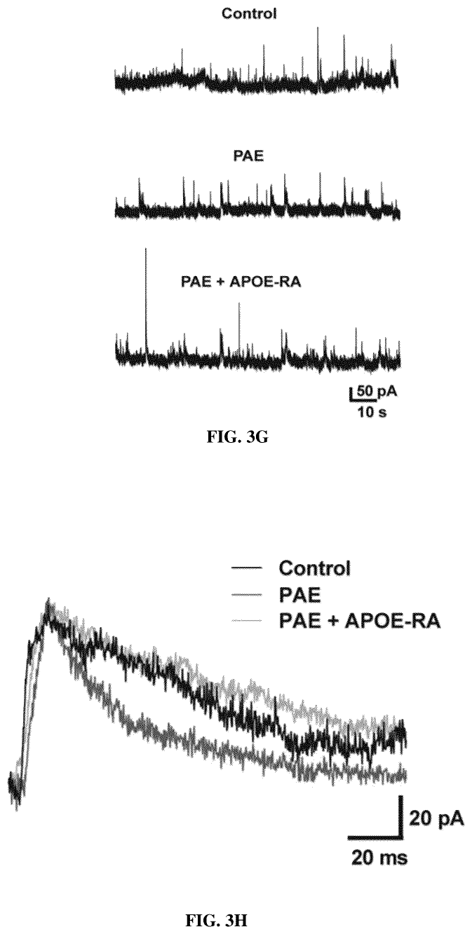

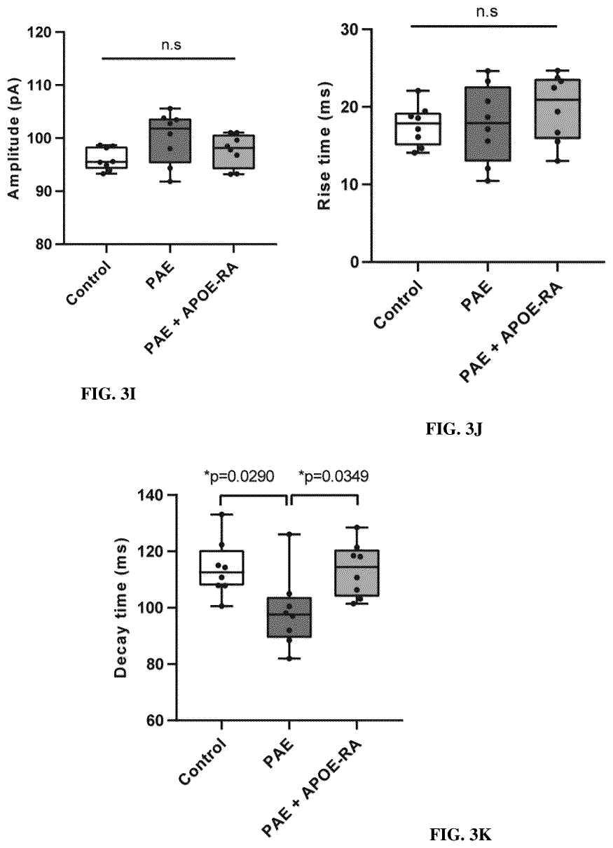

To test this hypothesis, it was first examined whether excitatory synaptic transmission via NMDARs and their kinetics are changed in PAE mice. Spontaneous NMDA excitatory postsynaptic currents (sNMDA EPSCs) were recorded in pyramidal neurons in layer V/VI of the motor cortex at P30. There was no difference in the amplitude and rise time in sNMDA EPSCs in PAE mice compared to control mice (FIGS. 3G-3J), indicating no changes in the synaptic response through NMDARs. However, a significantly shorter decay time in sNMDA EPSCs in PAE mice compared to control mice was found (FIGS. 3H and 3K). Consistent with the altered NMDAR kinetics, reduced downstream phosphorylation of cAMP-response element binding protein (CREB) (Deisseroth K et al. Neuron. 1996; 16:89-101; Impey S et al. Neuron. 2002; 34:235-44) in the motor cortex of PAE mice was observed (FIG. 14).

Treatment with APOE-RA for 10 days starting from P20 restored the shorter decay time in PAE mice to the level comparable to control mice (FIG. 3K), with no significant effects on the amplitude and rise time (FIGS. 3I-3J). Increased expression of KCNN2 in the motor cortex by PAE (Mohammad S et al. Nat Neurosci. 2020; 23:533-43) was also restored to the control levels after APOE-RA treatment (FIGS. 3C-3F). Double immunohistochemistry with KCNN2 and PSD-95 revealed the reduction of the PAE-induced increase in postsynaptic KCNN2 levels after the APOE-RA treatment (FIGS. 13G-13I). These results suggest that reduced APOE contributes to motor learning deficits in PAE mice by increasing KCNN2 expression and its synaptic level via altered NMDAR function.

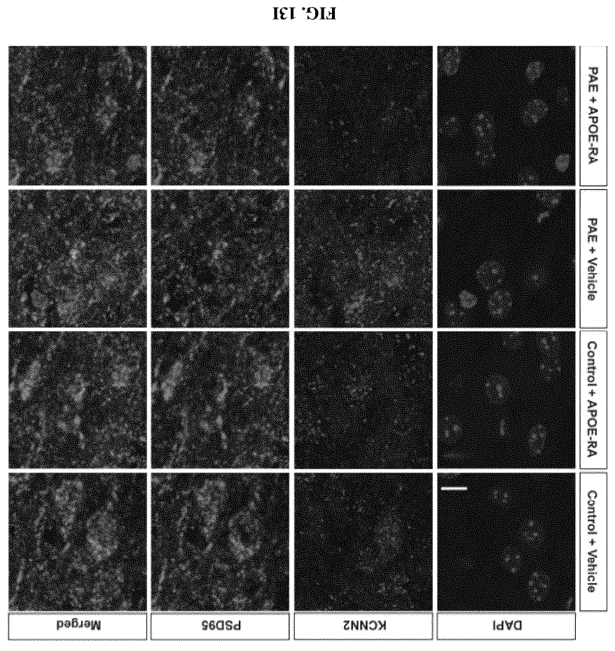

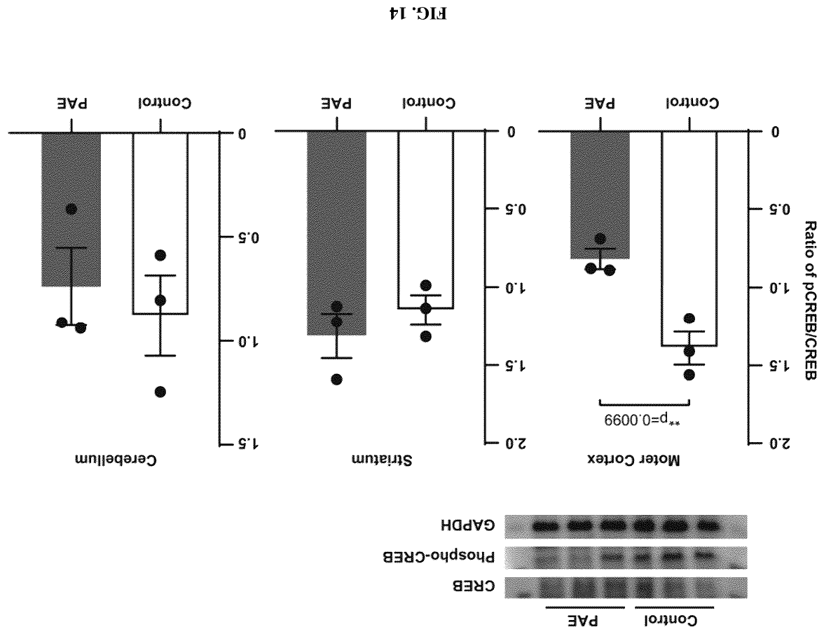

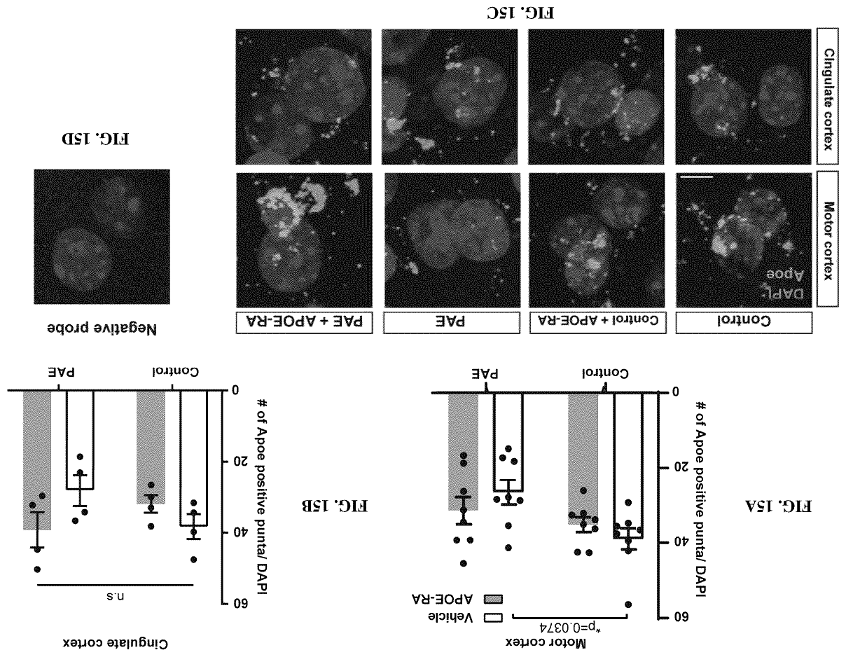

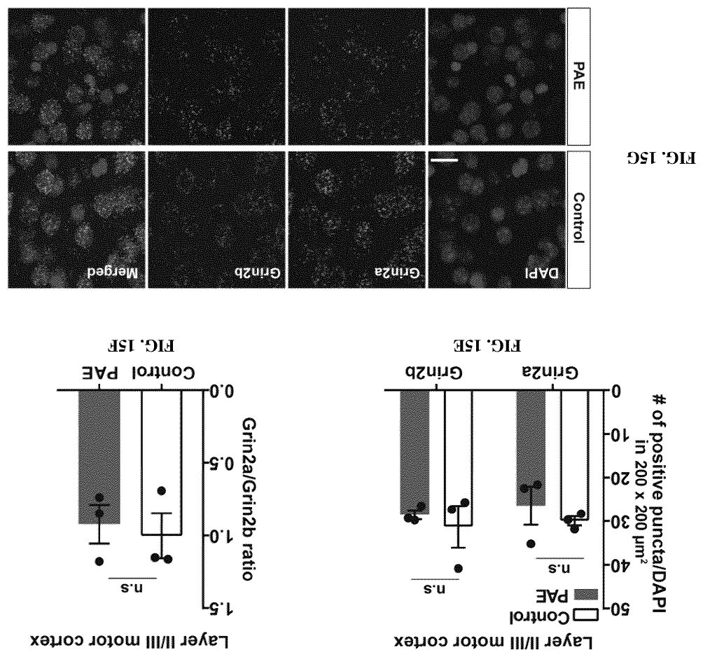

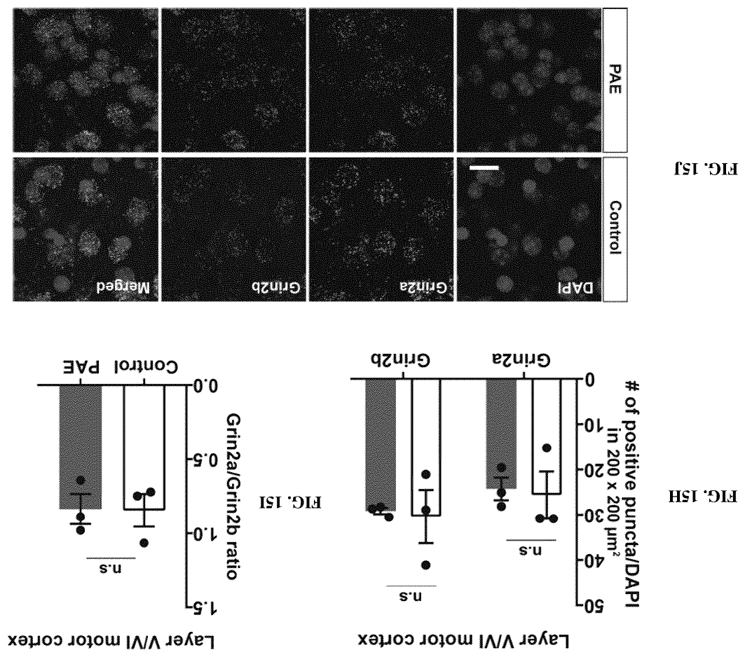

The decay time of sNMDA EPSCs is thought to shift from slow to fast during development by the change in the ratio between two NMDAR subunits GluN2A and GluN2B (Kirson E D et al. J Physiol. 1996; 497:437-55). However, no significant difference was observed in the expression of Grin2a and Grin2b (GluN2A and GluN2B encoding genes, respectively) in the motor cortex between control and PAE mice (FIGS. 15E-15J).

Decrease in Brain APOE in PAE Mice Involves Regulation at the Transcriptional Level, while APOE-RA Increases Brain APOE at the Post-Transcriptional Level

As reduction of Apoe mRNA in PBMC (FIGS. 1A-1H) and APOE protein in the motor cortex of PAE mice (FIGS. 3A-3B) was observed, the expression of Apoe mRNA in the motor cortex of PAE mice was examined. By utilizing a specific RNAscope probe, Apoe mRNA was detected around the nucleus (FIG. 15C), while a negative probe provided no signals as expected (FIG. 15D). Consistent with the reduced protein levels, reduction in the Apoe mRNA expression was observed in the motor cortex in PAE mice (FIGS. 15A-15C).

A previous study has shown that Ac-hE18A-NH2, an APOE mimetic peptide that has a similar structure to APOE-RA, increases endogenous brain APOE levels (Handattu S P et al. J Alzheimers Dis. 2013; 36:335-47). Thus, it was also tested whether endogenous APOE expression, which is decreased in the brain in PAE mice (FIGS. 3A-3K and FIGS. 9A-9H), was changed by APOE-RA administration. Based on the half-lives of similar APOE mimetic peptides [Ac-hE18A-NH2: <2 minutes for fast decay time and 90 min for long decay time (Garber D W et al. Atherosclerosis. 2003; 168:229-37); COG1410: 13±5 min (Kaufman N A et al. Behav Brain Res. 2010; 214:395-401), it was assumed that most of the APOE detected 24 h after the last dose of APOE-RA to be de novo endogenous APOE expression. Immunolabeling with an APOE antibody 24 hours after the final administration of the 10-day APOE-RA treatment showed an increase in APOE-positive cells in layer V/VI of the motor cortex in PAE mice compared to vehicle-treated PAE mice at P30 (FIGS. 3A-3B). A similar increase in APOE-expressing cells by APOE-RA treatment was also observed in the cingulate cortex in PAE mice (FIGS. 11E and 11F). In control mice prenatally exposed to PBS, it was confirmed no significant change in endogenous APOE expression by APOE-RA treatment in the motor and cingulate cortices (FIG. 3A and FIG. 11E). In contrast to the recovery of APOE protein level in PAE mice after APOE-RA treatment, no significant effect on the Apoe mRNA level was observed (FIGS. 15A-15C). Together, these results indicate that the reduction of brain APOE level in PAE mice involves its reduction at the transcriptional level, while APOE-RA treatment may increase the endogenous APOE level in PAE mice at the posttranscriptional level.

### Chromatin Accessibility at the ApoE Locus is Decreased in Motor Cortex of PAE Mice

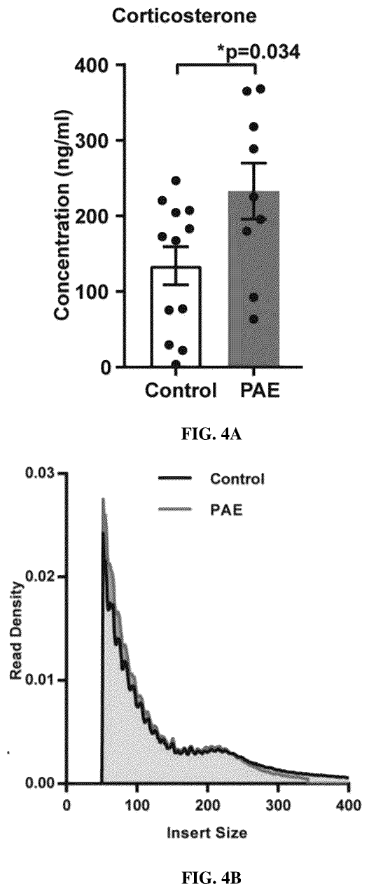

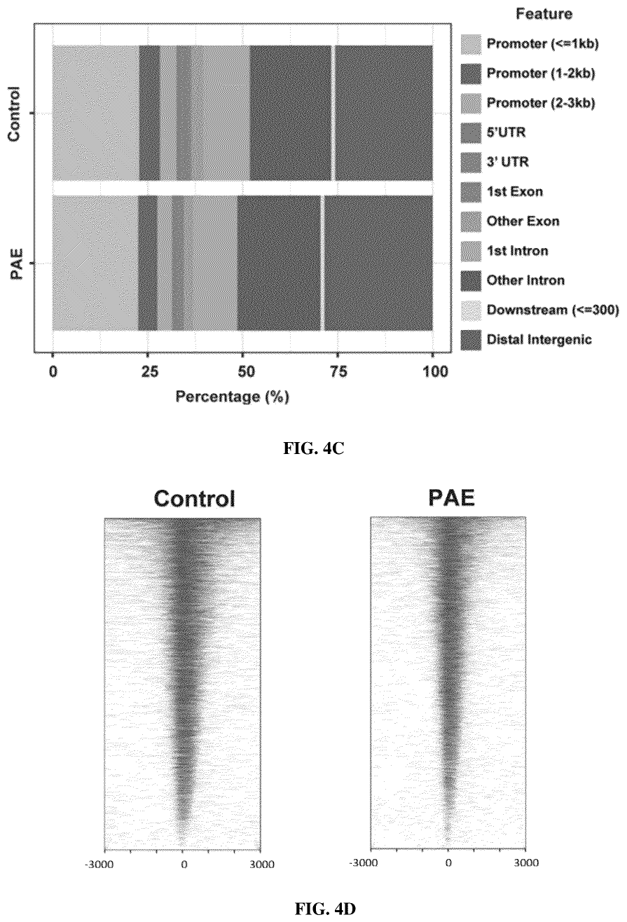

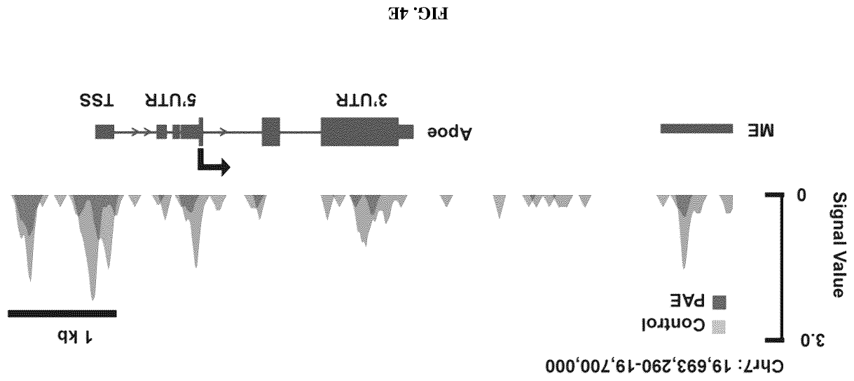

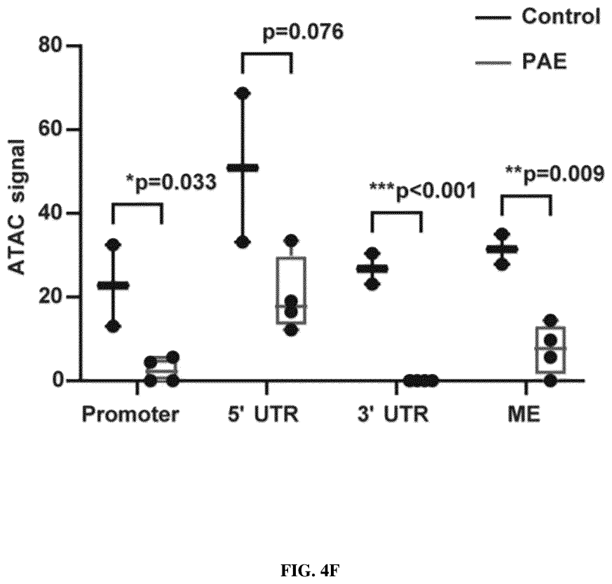

The Apoe regulatory region contains multiple glucocorticoid receptor binding loci (Kardassis D et al. Handb Exp Pharmacol. 2015; 224:113-79; Shih S J et al. J Biol Chem. 2000; 275:31567-72), and an increase in glucocorticoids enhances APOE gene expression by the direct binding on these loci (Kardassis D et al. Handb Exp Pharmacol. 2015; 224:113-79). In FASD children, the hypothalamic-pituitary-adrenal (HPA) axis has been shown to be hyperactive and increase cortisol levels (Keiver K et al. Alcohol. 2015; 49:79-87). Similarly, the PAE mice showed increased plasma corticosterone levels (FIG. 4A), which contradicted the decreased Apoe mRNA level in the brain (FIGS. 9A-9H).

A previous study demonstrated that chromatin structure changes in response to PAE and that these changes persist beyond the window of exposure (Veazey K J et al. Epigenetics Chromatin. 2015; 8:39). Therefore, it was hypothesized that Apoe gene transcription is epigenetically silenced by PAE. To test chromatin opening, the assay for transposase-accessible chromatin with sequencing (ATAC-seq) of pyramidal neurons in the motor cortex of PAE and control mice was performed. Neurons labelled with GFP introduced by in utero electroporation at E15 were collected by FACSorting. After Tn5 enzymatic reaction, it was confirmed that the majority of fragment sizes corresponded to nucleosome-free regions (<100 bp) and mono-nucleosome (˜200 bp) (FIG. 4B). Peak annotations were comparable between control and PAE groups (FIG. 4C). The density map also showed that ATAC peaks were enriched near the transcription starting site (TSS), confirming the quality of ATAC-seq (FIG. 4D). The signals around the Apoe locus between PAE and control was then quantified and compared. A significant reduction of ATAC signals was found in the promoter, 3′ UTR, and the multienhancer (ME), which controls Apoe promoter activity in different cell types (Trusca V G et al. J Biol Chem. 2011; 286:13891-904), in the PAE group (FIGS. 4E-4F), suggesting that the chromatin accessibility was reduced, and thereby Apoe expression was reduced in the motor cortex of PAE mice (FIG. 15A). These results indicate that the reduction of Apoe expression in the motor cortex is attributed to epigenetic effects of PAE.

A SNP in the APOE Enhancer is Associated with Impaired Neurocognition in Human Subjects in the Presence of PAE

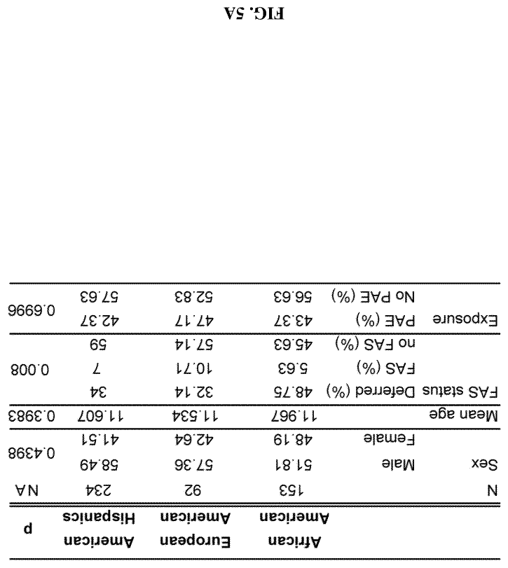

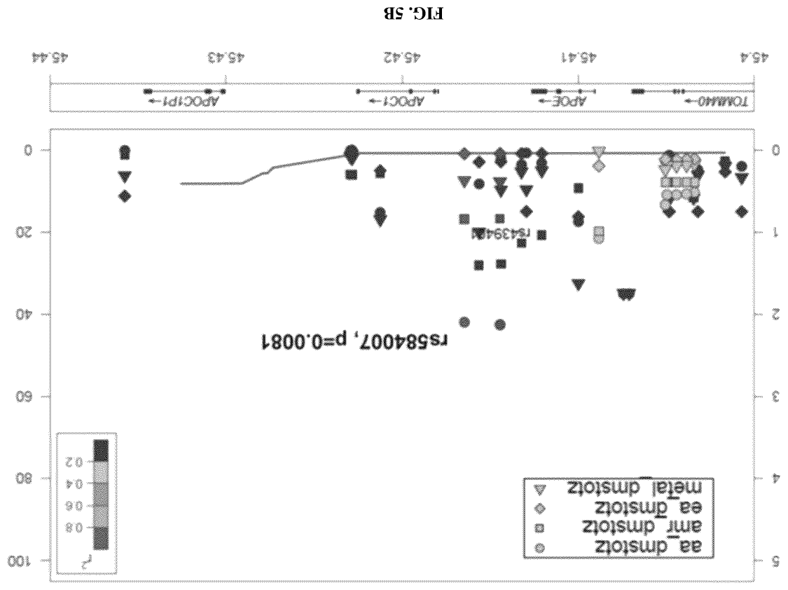

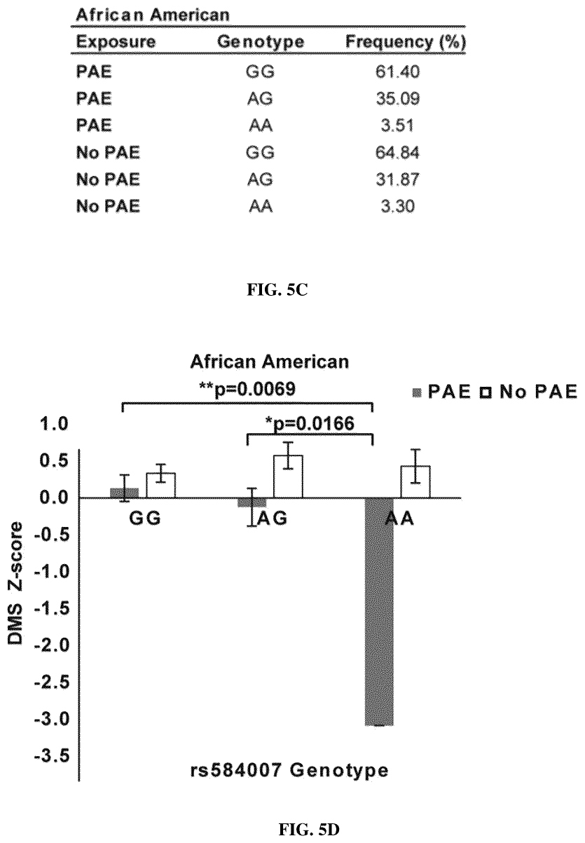

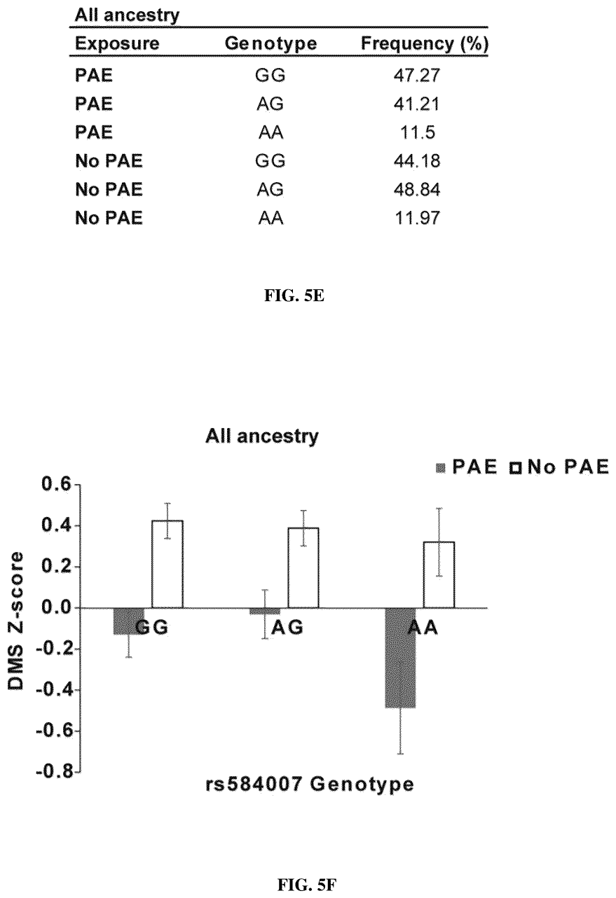

In humans, APOE level in the cerebrospinal fluid is known to be affected by genetic variants (Bekris L M et al. J Alzheimer's Dis. 2008; 13:255-66). Therefore, it was tested whether common variants (MAF≥0.01) around the APOE locus between 45.40 and 45.55 Mb on chromosome 19 were associated with neurobehavioral traits of PAE individuals genotyped as part of a genome-wide association study (GWAS). Saliva samples were collected from children with prenatal alcohol exposure and controls with minimal or no prenatal alcohol exposure of three ancestry groups (African Americans, European Americans, and American Hispanics) in the Collaborative Initiative on Fetal Alcohol Spectrum Disorders (CIFASD) US cohort (for more information on CIFASD methods, see Mattson S N, Jones K L, Chockalingam G, Wozniak J R, Hyland M T, Courchesne-Krak N S, et al. Validation of the FASD-Tree as a screening tool for fetal alcohol spectrum disorders. Alcohol. 2023; 47:263-72 and Mattson S N, Foroud T, Sowell E R, Jones K L, Coles C D, Fagerlund A, et al. Collaborative initiative on fetal alcohol spectrum disorders: methodology of clinical projects. Alcohol. 2010; 44:635-41). There were no significant differences between these three ancestry groups in the sex ratio, mean age, and presence of PAE (FIG. 5A). Within the group with PAE, which was recruited for participation in a study on the effects of PAE, the prevalence of FAS ranged 5-10%, with European Americans being the highest (FIG. 5A). While this prevalence is much higher than one previously reported (Roozen S et al. Alcohol Clin Exp Res. 2016; 40:18-32), CIFASD was not designed as a prevalence study and individuals with PAE were specifically recruited for participation.

The GWAS analysis revealed a significant (P=0.008) association between rs584007 genotype and PAE with the delayed matching to sample (DMS) Z-score in African Americans with PAE (aa_dmstotz in FIG. 5B). This SNP is located in the multienhancer (ME), where it was found that chromatin was closed by PAE in the mouse motor cortex (FIG. 4F). The DMS assessment is a cognitive test that measures short-term visual recognition memory, and a human fMRI study demonstrated that the task requires activation of both the prefrontal cortex, including the cingulate cortex, and the premotor area, which play important roles in decision-making and motor control, respectively (Daniel T A et al. Biol Psychol. 2016; 120:10-20). Within the group of African Americans with PAE, children with at least one A allele scored lower than GG. Although number of subjects is low, it was found that the genotype with a homozygous for rs584007 SNP (AA) interacts with PAE to yield the lowest performance in DMS test (FIGS. 5C-5D). When analysis was done between the rs584007 SNP genotype and DMS Z-score data of all three ancestry groups, a marginal reduction in the Z-score of AA-carrying PAE individuals was found (FIGS. 5E-5F). Allele frequency is not significantly different between ancestry groups. Together, these data suggest that PAE children who carry the SNP within the ME region have higher risk of low DMS Z-scores.

Reduced Plasma APOE Level is Associated with Lower Cognitive Performance in Infants with PAE

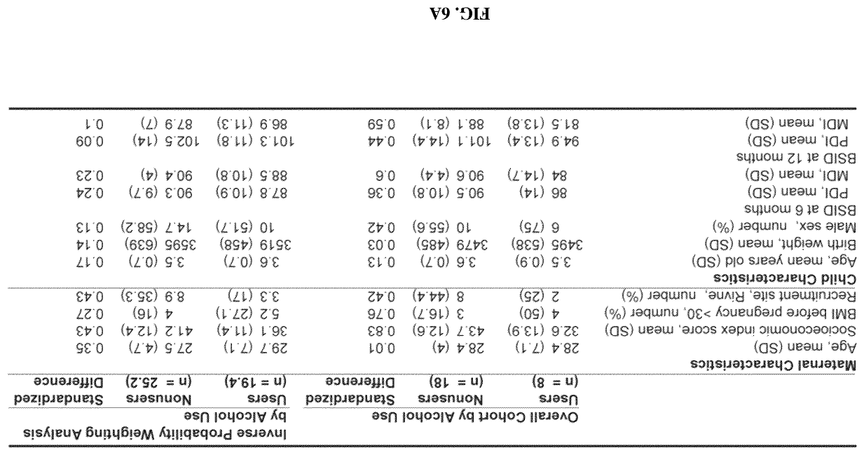

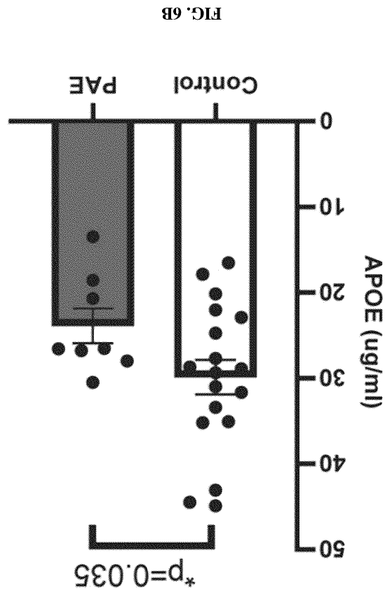

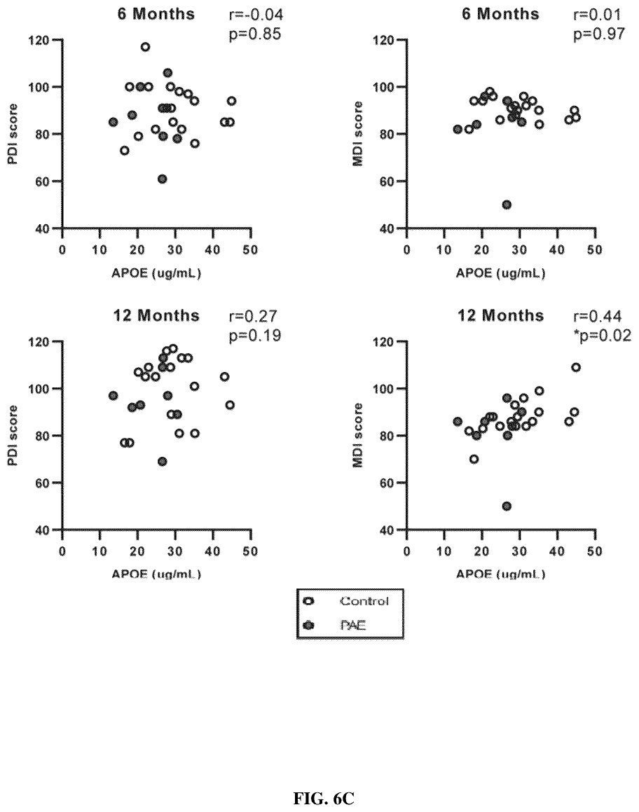

In an independent CIFASD cohort in Ukraine, plasma samples were collected from children with PAE and without aged 2-5 years old. In these subjects, plasma APOE levels and the relationship between these levels and neurodevelopmental outcomes were examined. By inverse probability weighting (IPW) with 4 maternal variables (age, socioeconomic index score, BMI before pregnancy, and recruitment site) and 4 child variables (age, birth weight, sex, and BSID-II) between two groups (FIG. 6A), it was found that plasma APOE levels in children with PAE were significantly lower than those in controls (FIG. 6B), as was observed in mice with PAE. Children in this cohort were also previously assessed for neurodevelopmental delay between 6 and 12 months of age, using the Bayley Scales of Infant Development (BSID-II), and specifically, the Psychomotor Development Index (PDI) and Mental Development Index (MDI). Pearson's correlation analysis indicated a significant correlation between child plasma APOE levels and previously obtained infant MDI scores (FIG. 6C).

### DISCUSSION

Utilizing a mouse model of FASD, this study discovered that APOE is a target for treatment and a peripheral biomarker for neurobehavior problems in FASD. Transcriptomics and immunohistological assays revealed a reduction of both peripheral and brain APOE in mice with PAE (FIGS. 1A-1H, 2A-2D, and 9A-9H). ATAC-seq data suggest that the reduction of chromatin accessibility around the Apoe locus in PAE mice is responsible for the reduction of APOE in the brain (FIGS. 4E-4F). The positive correlation between the APOE level and motor learning suggests that the decrease in APOE level contributes significantly to the deficits in motor skill learning in PAE mice (FIGS. 1G and 2A). Similarly, it was found that the plasma APOE level was reduced in PAE children, and the level was positively correlated with BSID-II scores at 12 months old (FIGS. 6A-6C). A peptide, APOE-RA, which mimics the APOE function as a ligand for its receptors, alleviated both the motor learning deficit and anxiety in PAE mice (FIGS. 2A-2D and FIGS. 11A-11J), demonstrating that appropriate levels of APOE receptor-mediated signaling are essential for normal performance in various behaviors.

While a blood alcohol concentration (BAC) was not measured in the Ukrainian cohort, a previous study has suggested that the BAC in pregnant individuals engaging in binge drinking, which is defined as consuming more than five standard drinks per occasion (same as the Ukrainian cohort), ranges from 110 to 370 mg/dl (Heller M et al. Birth Defects Res A Clin Mol Teratol. 2014; 100:277-83). Pregnant mice in the current study showed the BAC of 198-410 mg/dl within an hour after alcohol administration (Dutta D J et al. Proc Natl Acad Sci USA. 2023; 120: e2304074120), therefore, are expected to serve as a good model for these human subjects. However, since i.p. injection was used for alcohol administration to achieve precise dose control, these animals may have achieved target BAC levels more rapidly than human subjects (Almeida L et al. Front Pediatr. 2020; 8:359). Species differences, including that in the placental structure (Aguilera N et al. Anim Reprod. 2022; 19:e20210134), may also cause differences in alcohol metabolism during pregnancy.

Significant correlation of plasma APOE level with MDI but marginal correlation with PDI as observed (FIG. 6C). Marginal reduction of MDI score in PAE group at 6-(P=0.09 by Student's t-test) and 12-month-old (P=0.14), but not in PDI (P=0.38, 0.31 at 6, 12 month-old, respectively) was also detected. As seen in the mouse PAE model (Mohammad S et al. Nat Neurosci. 2020; 23:533-43) (FIG. 2C at trial 1), this data suggests that gross motor skill may be largely unaffected by PAE. As MDI shows higher correlation with cognitive indices at later stages in general population (Luttikhuizen dos Santos E S et al. Early Hum Dev. 2013; 89:487-96), the deficits in motor skill learning seen in mouse juvenile with PAE may be a consistent result with lower MDI in human infants with PAE. Lower APOE level was also observed in plasma of both mouse model and human children with PAE. The moderate correlation between the APOE level and MDI may reflect the limited number of subjects and/or the temporal difference in data collection (BSID-II was conducted in infancy, while plasma APOE levels were measured between 2 and 5 years old). Follow-up studies on later neurodevelopmental testing with these children is ongoing to further clarify the relationship between APOE and neurodevelopment.

The DMS assessment, a cognitive test that measures short-term recognition memory, is affected by interaction of a variant in APOE enhancer and PAE (FIG. 5D). Ablation of hippocampus does not change the performance in animals (Sloan H L et al. Behav Brain Res. 2006; 171:116-26), while other studies show contribution of various cortical and subcortical regions including premotor, primary motor and prefrontal cortices (Daniel T A et al. Biol Psychol. 2016; 120:10-20; Gobin C et al. Learn Mem. 2020; 27:467-76). These results suggest the contribution of broader circuitry in DMS task, and that the molecular mechanisms observed in motor cortex of mouse PAE may be applicable to other brain regions, altogether leading to the cognitive problems.

Paucity in data in human FASD cohort also limits the interpretation, especially APOE's contribution in anxiety phenotypes in mice with PAE. Consistent with the observation (FIGS. 11C and 11D), both increase of cortisol and anxiety had been observed in PAE human subjects as well as various animal models of PAE (Keiver K et al. Alcohol. 2015; 49:79-87; Osborn J A et al. Alcohol Clin Exp Res. 1998; 22:685-96, Hellemans K G C et al. Ann N Y Acad Sci. 2008; 1144:154-75). Although previous study showed crucial contribution of reduced neuronal activities in the anterior cingulate cortex in anxiety phenotype in the mouse with PAE (Hwang H M et al. Transl Psychiatry. 2022; 12:24), how APOE-RA affects activities in ACC to improve the phenotype remains still elusive.

The knowledge of the genetic factors that underlie vulnerabilities to FASD and alcohol teratogenicity is still in its infancy. Animal research and human epigenetic studies have shown that maternal and fetal genetic factors contribute to teratogenic risk of alcohol (Arfsten D P et al. Int J Toxicol. 2004; 23:47-54; Viljoen D L et al. Alcohol Clin Exp Res. 2001; 25:1719-22; Jacobson S W et al. J Pediatr. 2006; 148:30-37). Although the number of subjects is small as a nature of FASD cohort, the GWAS study suggests that the rs584007 SNP interacts with PAE in reducing DMS Z-scores in children (FIGS. 5D and 5F). The rs584007 SNP is located in the ME of ApoE locus where it was found that the chromatin is closed by PAE in mice (FIG. 5F).

ME region is known to crucially control the transcription of APOE in humans (Shih S J et al. J Biol Chem. 2000; 275:31567-72; Mak P A et al. J Biol Chem. 2002; 277:31900-8; Grehan S et al. J Neurosci. 2001; 21:812-22). Variants in the ME, including rs584007, have been shown to affect the APOE promoter activity and moderately decrease the APOE level in the cerebrospinal fluid in human adults (Bekris L M et al. J Alzheimer's Dis. 2008; 13:255-66; Bekris L M et al. J Hum Genet. 2012; 57:18-25). Furthermore, ME has been shown to physically interact with the APOE promoter in an anti-sense orientation in human macrophages and monocytes (Trusca V G et al. J Biol Chem. 2011; 286:13891-904), suggesting that variants in the ME may affect APOE promoter activity through direct interactions with the promoter to modulate APOE expression. These previous studies and the present results collectively suggest that the genetic predisposition in the APOE enhancer exacerbates PAE-caused reduction of APOE expression, and thereby increases the risk of eliciting neurobehavioral problems.

Besides transcriptional regulation at the APOE locus, other mechanisms are possibly involved in APOE expression and distribution. For instance, a paper has reported that the choroid plexus/CSF provides an additional source of APOE, and the glymphatic fluid transporting system delivers APOE to the brain via the periarterial space (Achariyar T M et al. Mol Neurodegener. 2016; 11:74). Interestingly, reduced inflow of APOE-containing CSF by sleep deprivation has been shown (Achariyar T M et al. Mol Neurodegener. 2016; 11:74). As sleep deprivation in FASD patients has been reported (Chen M L et al. J Clin Sleep Med. 2012; 8:421-9; Dylag K A et al. Ital J Pediatr. 2021; 47:113), this mechanism may be involved in APOE reduction by PAE.

The APOE level was decreased in the motor cortex but not in the cingulate cortex in PAE mice (FIG. 2A and FIGS. 9A-911). Region specific regulation of endogenous APOE, e.g., high expression of APOE mRNA in neurons in the frontal cortex and hippocampus while little expression in the cerebellar cortex in the human brain (Xu P-T et al. Am J Pathol. 1999; 154:601-11), may underlie such region-specific alteration by PAE. As an acute prenatal exposure model was used, it is possible that the regional specificity also depends upon the timing and dosage of alcohol exposure. The correlation between the APOE level in the motor cortex and motor learning index (FIG. 2A) is consistent with the fact that the motor cortex plays a crucial role in motor learning (Yang G et al. Nature. 2009; 462:920-4; Cichon J et al. Nature. 2015; 520:180-5; Biane J S et al. Neuron. 2016; 89:1173-9; Li Q et al. Nat Commun. 2017; 8:15834). Mice with PAE exhibit anxiety behavior, which is attributed to the reduced activity of the cingulate cortex (Hwang H M et al. Transl Psychiatry. 2022; 12:24). Although increased expression of endogenous APOE was observed in the cingulate cortex in mice with PAE by APOE-RA administration, which mitigated the anxiety behavior (FIGS. 11A-11J), no significant decrease was observed in the APOE level in the cingulate cortex in mice with PAE (FIGS. 9A-911). The reduced activity of the cingulate cortex and anxiety behavior in mice with PAE, therefore, may depend on reduced APOE level in brain regions other than the cingulate cortex. Lower levels of plasma APOE were found both in the mouse model of acute PAE (FIGS. 1A-1H) and in children with PAE (FIGS. 6A-6C), who were likely periodically exposed to alcohol throughout gestation. Lower levels of Apoe mRNA were also found in PBMCs of mice with PAE (FIGS. 1A-1H). These results also suggest that APOE levels in blood samples may serve as a biomarker for neurobehavioral deficits associated with PAE, regardless of acute or chronic exposure. Brain specific Apoe conditional KO mice show a reduction in the number of synapses and dendrites and AMPA/NMDA ratio, and infusion of recombinant APOE into lateral ventricles or genetic restoration of Apoe improves the phenotypes (Lane-Donovan C et al. J Neurosci. 2016; 36:10141-50; Masliah E et al. Brain Res. 1997; 751:307-14). In the present study, the effects of a single dose of daily treatment with APOE-RA for 10 days on PAE mice were tested. Similar to the previous finding that administration of a small APOE mimetic peptide Ac-hE18A-NH2 increases endogenous APOE in the mouse brain (Handattu S P et al. J Alzheimers Dis. 2013; 36:335-47), an increase in endogenous APOE in the brain after 10 days of APOE-RA administration to PAE mice was observed (FIG. 4A and FIG. 11E). Thus, the enhancement of intrinsic mechanism may augment the effects of APOE-RA treatment in alleviating the phenotype in PAE mice. In this regard, a recent study has shown that APOE can translocate to the nucleus and bind to DNA to function as a transcription factor in human glioblastoma cells (Theendakara V et al. J Neurosci. 2016; 36:685-700). Surprisingly, however, the increase of endogenous APOE expression is not through the transcriptional control that is compromised by PAE (FIGS. 15A-15J). How APOE-RA increases APOE at the posttranscriptional level awaits further study. Given malfunctioning metabolism in FASD (Weeks O et al. J Clin Investig. 2020; 130:2252-69) and the increase of endogeneous APOE expression by APOE-RA (FIGS. 15A-15J), it was not possible to exclude the possibility that APOE-RA improves neurobehavioral problems of mice with PAE through improvement of whole body metabolism in addition to the mechanism in which APOE-RA directly binds to receptors in brain as discussed the detail below.

LRP1 is a member of the LDL receptor family (Auderset L et al. Stem Cells Int. 2016; 2016:1-16) that is highly expressed in neurons (Rebeck G W et al. Neuron. 1993; 11:575-80). Cortical neuron-specific conditional knockout of Lrpl results in severe loss of motor function (both learning and locomotor) without histologically detectable structural abnormalities in the brain or effects on LTP and other major electrophysiological properties (Liu Q et al. J Neurosci. 2010; 30:17068-78; May P et al. Mol Cell Biol. 2004; 24:8872-83). A study has demonstrated that LRP1 is preferentially recognized by lipid-free recombinant APOE compared to other LDL receptor families (Narita M et al. J Biochem. 2002; 132:743-9). Therefore, it is likely that the functions of APOE and APOE-RA in the neurobehavior that were observed are mediated by their binding to LRP1 (Croy J E et al. Biochemistry. 2004; 43:7328-35). Nevertheless, since APOE also binds to other LDL receptors including ApoER2 and VLDLR that also interact with PSD-95 at the postsynaptic terminals and play a role in synaptic plasticity (Lane-Donovan C et al. J Lipid Res. 2017; 58:1036-43), the relative contribution of each receptor to these functions of APOE and APOE-RA needs to be formally tested.

The electrophysiological recording revealed that the sNMDA EPSC decay time in the motor cortex of PAE mice was significantly shorter and was restored by APOE-RA treatment to a level comparable to that of control mice (FIG. 3K). Given that a shorter decay time of sNMDA EPSC is known to be associated with reduced neuronal plasticity (Brigman J L et al. J Neurosci. 2010; 30:4590-4600), the change in the kinetic property of NMDAR may be associated with the motor learning abilities. As it was also observed reduction of CREB phosphorylation, which occurs in the downstream of NMDAR signaling and mediates transcription of many genes (Deisseroth K et al. Neuron. 1996; 16:89-101; Impey S, Fong A L, Wang Y, Cardinaux J-R, Fass D M, Obrietan K, et al. Phosphorylation of CBP mediates transcriptional activation by neural activity and CaM kinase IV. Neuron. 2002; 34:235-44), in the motor cortex by PAE (FIG. 14), aberrant transcription of downstream genes involved in neuronal plasticity may also be involved in neurobehavioral problems in PAE.

Previous studies have shown that the levels of GluN2A and GluN2B subunits are associated with NMDAR decay kinetics (Vicini S et al. J Neurophysiol. 1998; 79:555-66) and developmental plasticity (Quinlan E M et al. Nat Neurosci. 1999; 2:352-7; Erisir A et al. J Neurosci. 2003; 23:5208-18), and the change in Grin2b to Grin2a expression ratio underlies the developmental shift of NMDAR decay time from slow to fast (Kirson E D et al. J Physiol. 1996; 497:437-55). Indeed, overexpression of GluN2A has been shown to cause faster decay time than normal in cerebellar granule cells without affecting the amplitude of NMDA EPSC (Prybylowski K et al. J Neurosci. 2002; 22:8902-10) similar to what was observed in the PAE mice (FIGS. 3I and 3K). Enhanced GluN2A expression has also been associated with impaired long term synaptic plasticity, and learning and memory in mice (Holehonnur R et al. J Neurosci. 2016; 36:9490-504; Li Q-Q et al. Mol Psychiatry. 2022; 27:3468-78). However, significant difference in the expression of Grin2a and Grin2b in the motor cortex between control and PAE mice was not observed (FIGS. 15E-15J). It is possible that the GluN2A to GluN2B ratio changes at the post-transcriptional level. Alternatively, but not mutually exclusively, since LRP1 has been shown to control the surface distribution and internalization of GluN2B in cortical neurons (Maier W et al. Mol Neurodegener. 2013; 8:25), intracellular trafficking and recycling of these NMDAR subunits may be affected by PAE and likewise by APOE-RA administration. In connection with this, chronic voluntary drinking during pregnancy has been shown to decrease synaptic GluN2B in the dentate gyrus in mouse offspring (Brady M L et al. J Neurosci. 2013; 33:1062-7), while in another study, increase of GluN2B by PAE using similar regimen has been reported in the insular cortex of rat offspring (Bird C W et al. PLoS ONE. 2015; 10:e0118721). The difference in these two studies may be due to the differences in the brain regions observed and/or alcohol dosages used: 5% (Brady M L et al. 2013; 33:1062-7) vs. 10% (Bird C W et al. PLoS ONE. 2015; 10:e0118721). Further studies are needed to address whether the synaptic GluN2A and GluN2B protein levels are altered in cortical neurons by PAE.

Upregulation of KCNN2, which leads to increased medium afterhyperpolarization in cortical pyramidal neurons and directly contributes to motor learning deficits in PAE mice (Mohammad S et al. Nat Neurosci. 2020; 23:533-43) is suppressed by APOE-RA treatment (FIGS. 3C-F and FIG. 11G). KCNN2 is degraded via the ubiquitin-proteasome pathway (Sun J et al. Sci Rep. 2020; 10:9824), which depends on NMDAR-mediated activation of calcium/calmodulin-dependent protein kinase II (CaMKII) in synaptic plasticity (Jarome T J et al. 2013; 105:107-16), while the effect of KCNN2 on CaMKII localization is required to refine synaptic plasticity (Shrestha A et al. Front Synaptic Neurosci. 2019; 11:18). Therefore, dysregulation of this mutual interaction of KCNN2 and NMDAR pathways due to APOE reduction may also be one of the mechanisms contributing to the motor learning deficits in PAE mice.

In conclusion, this study identified a mechanism by which PAE causes a decrease in the APOE level in the brain through an epigenetic mechanism, leading to neurobehavioral deficits. Although the number of patient samples is still small, the discovery of a PAE-susceptible SNP in the APOE enhancer and the finding of reduced plasma APOE levels, associated with lower cognitive performance in PAE individuals provide evidence for a genetic predisposition that interacts with PAE for the first time, and strongly support clinical translatability of APOE-targeted treatments for FASD.

### Example 2: Nasal Administration of APOE-RA

This example provides data demonstrating that intranasal administration of the APOE receptor agonist (APOE-RA), COG-133, improves impaired motor skill learning in prenatal alcohol exposure (PAE) mice.

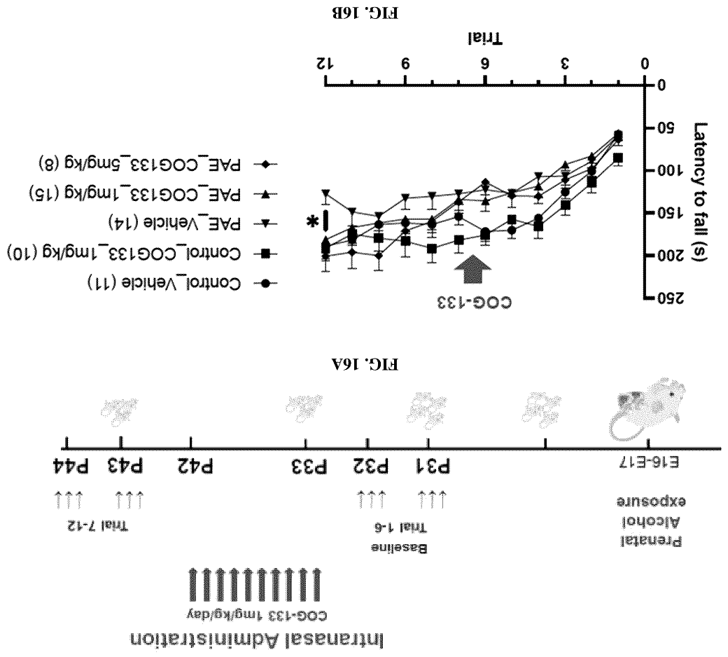

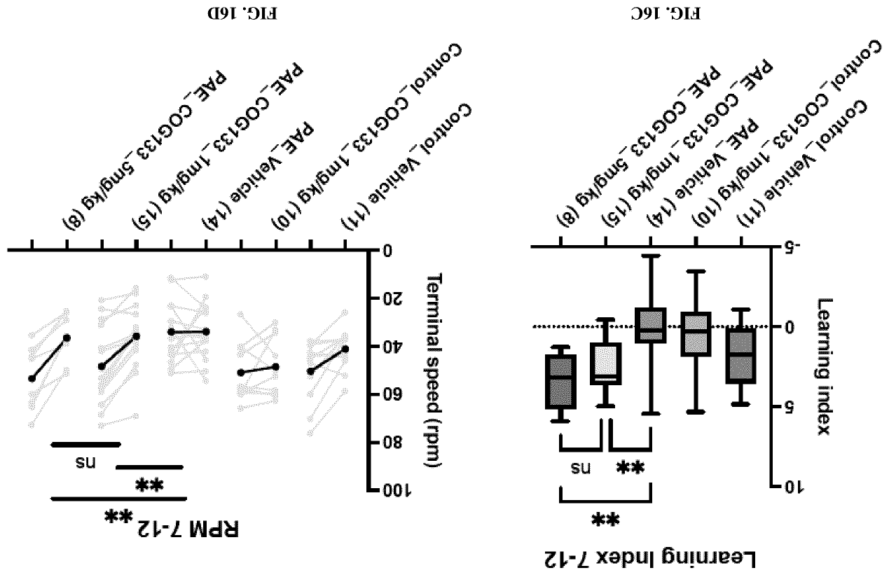

Briefly, mice were prenatally exposed to alcohol (4 g/kg) at embryonic day (E) 16 and 17, and the effect of intranasal administration of COG-133 (1 or 5 mg/kg) for 10 days after 6 trials on the accelerated rotarod was analyzed. FIG. 16A provides a schematic of the experimental time course.

The results of this experiment are provided in FIGS. 16B-16D. As shown in FIG. 16B, the interaction between the effects of treatment with COG-133 or Vehicle (PBS) and trial on the performance in the rotarod test is significant in PAE mice. COG-133-treated PAE mice show significantly shorter latency to fall from later trials. No significant effects of COG-133 were observed in the control mice. As shown in FIG. 16C, COG-133-treated PAE mice showed significant improvement in learning index from trial 7 to 12 compared to vehicle-treated PAE mice. As shown in FIG. 16D, COG-133-treated PAE mice show significant improvement in terminal speed from trial 7 to 12 compared to vehicle-treated PAE mice.

Thus, COG-133 administered intranasally improves impaired motor skill learning in PAE mice.

1. A method for treating or alleviating a fetal alcohol spectrum disorder (FASD) in a subject, the method comprising administering to the subject an effective amount of an apolipoprotein E (APOE)-modulating therapeutic, thereby treating or alleviating the FASD.

2. The method of claim 1, wherein the APOE-modulating therapeutic is selected from the group consisting of a retinoid X receptor (RXR) agonist, a liver X receptor (LXR) agonist, and an APOE-mimetic therapeutic.

3. The method of claim 2, wherein the APOE-modulating therapeutic is an APOE-mimetic therapeutic.

4. A method for treating or alleviating an FASD in a subject, the method comprising administering to the subject an effective amount of an APOE-mimetic therapeutic, thereby treating or alleviating the FASD.

5. The method of claim 1, wherein the FASD is selected from the group consisting of fetal alcohol syndrome (FAS), partial fetal alcohol syndrome (pFAS), alcohol-related neurodevelopmental disorder (ARND), and neurobehavioral disorder associated with prenatal alcohol exposure (ND-PAE).

6. The method of claim 3, wherein the APOE-mimetic therapeutic is selected from the group consisting of COG133, COG1410, COG112, CN-105, Ac-hE18A-NH₂, AEM-28(R), AES2-21, mR18L, EpK, hEp, CS-6253, ApoE(130-149), ApoE(141-155)₂, SP16, and Angiopep-2.

7. The method of claim 6, wherein the APOE-mimetic therapeutic is COG133.

8. The method of claim 1, wherein the subject carries a mutation in an APOE enhancer element.

9. The method of claim 8, wherein the mutation is a single nucleotide polymorphism (SNP).

10. The method of claim 9, wherein the SNP is rs584007.

11. The method of claim 10, wherein rs584007 comprises the amino acid sequence of
(SEQ ID NO: 16)
AGGAGGGGCGTCAGAGGGTGAATAARAGCAGATAGAGTGTTTGGGGGAGGT.

12. The method of claim 1, wherein the subject has low APOE plasma levels, as compared to APOE plasma levels of an individual without prenatal alcohol exposure (PAE).

13. The method of claim 1, wherein the subject has low APOE plasma levels, as compared to APOE plasma levels of an individual without the FASD.

14. The method of claim 1, wherein treating or alleviating the FASD comprises treating or alleviating one or more symptoms selected from the group consisting of a learning deficit, motor deficit, anxiety, depression, poor reasoning and judgement, short attention span, hyperactive behavior, poor memory, and speech delay.

15. The method of claim 1, wherein the subject is a human subject.

16. The method of claim 1, wherein the subject is a pediatric subject.

17. The method of claim 1, wherein the subject is an infant.

18. The method of claim 1, wherein the APOE-mimetic therapeutic is administered intranasally or intravenously.

19. The method of claim 18, wherein the APOE-mimetic therapeutic is administered intranasally.

20. A method of increasing APOE activity in a subject, the method comprising administering to the subject an effective amount of an APOE-modulating therapeutic, thereby increasing APOE activity, wherein the subject has or is suspected of having PAE.

21. A method of identifying a subject having an FASD, the method comprising: (a) measuring the level of APOE in a biological sample from the subject, and (b) comparing the measured level of APOE to a control level, wherein a lower level of APOE in the biological sample from the subject compared to the control level is indicative of the subject having the FASD, optionally, the method further comprising selecting or recommending the subject for treatment of FASD with the method of claim 1, and further optionally treating the subject having FASD with the method of claim 1.

22. The method of claim 21, wherein the biological sample is a plasma sample.


SEQUENCE LISTING (16 sequences)

SEQ ID NO: 1 - protein, 17 aa, synthetic construct
  MOD_RES 1: note = N-terminal acetylation
  MOD_RES 17: note = C-terminal amidation
LRVRLASHLRKLRKRLL

SEQ ID NO: 2 - protein, 12 aa, synthetic construct
  MOD_RES 1: note = N-terminal acetylation
  MOD_RES 3: note = alpha-aminoisobutyric acid (Aib)
  MOD_RES 8: note = alpha-aminoisobutyric acid (Aib)
  MOD_RES 12: note = C-terminal amidation
ASXLRKLXKRLL

SEQ ID NO: 3 - protein, 34 aa, synthetic construct
  MOD_RES 1: note = N-terminal acetylation
  MOD_RES 34: note = C-terminal amidation
RQIKIWFQNRRMKWKKCLRVRLASHLRKLRKRLL

SEQ ID NO: 4 - protein, 5 aa, synthetic construct
  MOD_RES 1: note = N-terminal acetylation
  MOD_RES 5: note = C-terminal amidation
VSRRR

SEQ ID NO: 5 - protein, 28 aa, synthetic construct
  MOD_RES 1: note = N-terminal acetylation
  MOD_RES 28: note = C-terminal amidation
LRKLRKRLLRDWLKAFYDKVAEKLKEAF

SEQ ID NO: 6 - protein, 28 aa, synthetic construct
  MOD_RES 1: note = N-terminal acetylation
  MOD_RES 28: note = C-terminal amidation
LRRLRRRLLRDWLKAFYDKVAEKLKEAF

SEQ ID NO: 7 - protein, 28 aa, synthetic construct
  MOD_RES 1: note = N-terminal acetylation and 6-aminohexanoic acid spacer (Ahx)
  MOD_RES 28: note = C-terminal amidation
LRRLRRRLLRDWLKAFYDKVAEKLKEAF

SEQ ID NO: 8 - protein, 18 aa, synthetic construct
  MOD_RES 1: note = N-terminal acetylation
  MOD_RES 18: note = C-terminal amidation
GFRRFLGSWARIYRAFVG

SEQ ID NO: 9 - protein, 38 aa, synthetic construct
CLRKLRKRLLRKKKKKKQVAEVRAKLEEQAQQIRLQAE

SEQ ID NO: 10 - protein, 61 aa, synthetic construct
EELRVRLASHLRKLRKRLLRDADDLQKRLAVYEEQAQQIRLQAEAFQARLKSWFEPLVEDM

SEQ ID NO: 11 - protein, 26 aa, synthetic construct
  MOD_RES 3: note = citrulline (Cit)
  MOD_RES 14: note = citrulline (Cit)
EVXSKLEEWLAALXELAEELLARLKS

SEQ ID NO: 12 - protein, 20 aa, synthetic construct
CGRLVQYRGEVQAMLGQSTE

SEQ ID NO: 13 - protein, 15 aa, synthetic construct
QAMLGQSTEELRVRL

SEQ ID NO: 14 - protein, 17 aa, synthetic construct
VKFNKPFVFLMIEQNTK

SEQ ID NO: 15 - protein, 19 aa, synthetic construct
TFFYGGSRGKRNNFKTEEY

SEQ ID NO: 16 - DNA, 51 bp, synthetic construct
  source 1..51: mol_type = other DNA
aggaggggcgtcagagggtgaataaragcagatagagtgtttgggggaggt